(19) United States
(12) Patent Application Publication
(10) Pub. No.: US 2025/0290111 A1
(43) Pub. Date: Sep. 18, 2025

(54) ANCESTRAL PROTEIN SEQUENCES AND PRODUCTION THEREOF

(71) Applicant: Per-Olof SYRÉN, Sundbyberg (SE)

(72) Inventors: Karen SCHRIEVER, Solna (SE); Juni ANDRÉLL, Vällingby (SE); Per-Olof SYRÉN, Sundbyberg (SE); David Alexander HUETING, Bromma (SE)

(21) Appl. No.: 18/862,233

(22) PCT Filed: May 3, 2023
(86) PCT No.: PCT/SE2023/050423

§ 371 Date: Nov. 1, 2024

(30) Foreign Application Priority Data
May 3, 2022 (SE) 2250542-4

(51) Int. Cl. C12P 21/02; C07K 14/005

(52) U.S. Cl. CPC C12P 21/02; C07K 14/005; C12N 2770/20022; C12N 2770/20051

(57) ABSTRACT

A protein, such as an antigenic protein, is produced by determining an amino acid sequence of an ancestral version of a given protein in an ancestral sequence reconstruction method based on a plurality of homologous amino acid sequences of the given protein. A domain of the amino acid sequence of the ancestral version of the given protein is replaced with a corresponding domain derived from an amino acid sequence of the given protein or a homologous version thereof. The protein thereby comprises the amino acid sequence obtained by replacing the domain of the amino acid sequence of the ancestral version of the given protein with the corresponding domain derived from the amino acid sequence of the given protein or the homologous version thereof. The protein is suitable as antigen, as vaccine candidate and/or for structural studies.

### TECHNICAL FIELD

The present invention generally relates to ancestral sequence reconstruction, and in particular to the production of ancestral protein sequences suitable as antigens, as vaccine candidates and/or for structural studies.

### BACKGROUND

Severe acute respiratory syndrome coronavirus 2 (SARS-CoV-2) was identified in 2019 as a new virus causing a pneumonia outbreak in Wuhan, China. The disease (coronavirus disease 2019, COVID-19) has had devastating consequences on global health and economy and in March 2020, the World Health Organization (WHO) declared it a pandemic. Up to now, SARS-CoV-2 has resulted in million deaths globally. To enable entrance into the host cell, SARS-CoV-2 and other coronaviruses employ a fusion protein referred to as spike protein (S protein), a homotrimeric glycosylated ectodomain consisting of a receptor binding subunit (S1) and membrane fusion subunit (S2). The spike protein associates with a host cell receptor, the angiotensin converting enzyme 2 (ACE2). The spike protein comprises highly flexible parts that assist in conformational switching from the so called “down” and closed receptor inaccessible state to the “up” and open state for engagement with the ACE2 receptor. The spike protein is a key target for antibody binding and vaccine development, including an epitope within the so-called receptor binding domain (RBD) corresponding to residues 319-541 for SARS-CoV-2.

However, low stability and low titers upon protein expression are obstacles that pose challenges to development of a low-cost vaccine based on the spike protein that could be administered in room temperature globally. The state-of-the art approach to solve the stability problem lies in low-throughput structural-guided single amino acid substitutions to stabilize the spike protein, which has resulted, among others, in the so-called HexaPro construct with six proline mutations.

Furthermore, the SARS-CoV-2 virus undergoes expedient evolution. Mechanisms of animal-to-human transmission contribute in generating spike protein and virus diversity. Mutated strains of SARS-CoV-2 with multiple substitutions in the spike protein frequently appear and could possibly evade immune responses, which is of increasing global concern. For instance, variants originating from the UK (alpha variant), South Africa (beta variant), Brazil (gamma variant), India (delta variant) and the omicron variant can display enhanced infectability, increased mortality rates and/or elevated antibody resistance. The efficiency of already existing antibodies generated from recovered and/or vaccinated patients in neutralizing these rapidly spreading SARS-CoV-2 strains was found to decrease.

A similar problem exists also for other rapidly evolving viruses, such a human immunodeficiency virus (HIV).

Selberg 2021 summarizes applications of ancestral sequence reconstruction within biomedical applications and biotechnology.

Gaschen 2002 suggests that consensus or ancestor sequences could be used in vaccine design against HIV-type 1 (HIV-1) to minimize the genetic differences between vaccine strains contemporary isolates.

Ducatez 2011 reconstructed ancestral protein sequences at several nodes of the hemagglutinin (HA) and neuraminidase (NA) gene phylogenies that represent ancestors to diverse H5N1 influenza virus clades. A panel of replication competent influenza viruses containing synthesized HA and NA genes representing the reconstructed ancestral proteins was produced and tested as whole-virus vaccines.

US 2002/0137094 discloses a method for improving thermostability of proteins. The method comprises (i) comparing amino acid sequences of proteins derived from two or more species which evolutionarily correspond to each other in a phylogenetic tree, (ii) estimating an amino acid sequence of an ancestral protein corresponding to the amino acid sequences compared in step (i), (iii) comparing the amino acid residues in the amino acid sequence in one of the proteins compared in step (i) with amino acid residues at a corresponding position in the ancestral protein estimated in step (ii), and (iv) replacing one or more of the amino acid residues different from those of the ancestral protein with the same amino acid residues as those of the ancestral protein.

There is still a need for the production of pathogen proteins in high titers that are stable and that could be used in the generation of vaccines and antigens.

### SUMMARY

It is a general objective to provide ancestral protein sequences suitable as antigens, as vaccine candidates and/or for structural studies.

This and other objectives are met by embodiments as disclosed herein.

The present invention is defined in the independent claims. Further embodiments of the invention are defined in the dependent claims.

An aspect of the invention relates to a coronavirus spike protein comprising an amino acid sequence according to the formula Seq1-RBD-Seq2. Seq1 comprises an amino acid sequence selected from the group consisting of SEQ ID NO: 5, 15, 25 and an amino acid sequence having at least 90% sequence identity, preferably at least 95% sequence identity, and more preferably at least 97% sequence identity to any of SEQ ID NO: 5, 15 and 25. RBD represents a receptor binding domain. Seq2 comprises an amino acid sequence selected from the group consisting of SEQ ID NO: 8, 18, 28 and an amino acid sequence having at least 90% sequence identity, preferably at least 95% sequence identity, and more preferably at least 97% sequence identity to any of SEQ ID NO: 8, 18 and 28.

Further aspects of the invention relate to a nucleic acid molecule encoding a coronavirus spike protein according to above, an expression vector comprising a nucleic acid molecule according to above and a host cell comprising an expression vector according to above.

Other aspects of the invention relate to a coronavirus spike protein or a nucleic acid molecule according to above for use as a vaccine or for use in prevention or treatment of a coronavirus infection or a coronavirus infectious disease.

Another aspect of the invention relates to a protein production method. The method comprises providing a plurality of homologous amino acid sequences of a given protein. The method also comprises determining an amino acid sequence of an ancestral version of the given protein in an ancestral sequence reconstruction method based on the plurality of homologous amino acid sequences of the given protein. The method further comprises replacing a domain of the amino acid sequence of the ancestral version of the given protein with a corresponding domain derived from an amino acid sequence of the given protein or a homologous version thereof. The method additionally comprises producing a protein comprising the amino acid sequence obtained by replacing the domain of the amino acid sequence of the ancestral version of the given protein with the corresponding domain derived from the amino acid sequence of the given protein or the homologous version thereof.

The present invention provides a method of producing proteins that are robust and can be produced in high titer. The proteins are suitable as antigens and vaccine candidates and/or for conducting structural studies.

### BRIEF DESCRIPTION OF THE DRAWINGS

The embodiments, together with further objects and advantages thereof, may best be understood by making reference to the following description taken together with the accompanying drawings, in which:

FIG. 1 is a flow chart illustrating a method of producing a protein according to an embodiment.

FIG. 2 is a flow chart illustrating an embodiment of the providing step in FIG. 1.

FIG. 3 is a flow chart illustrating additional, optional steps of the method shown in FIG. 2 according to various embodiments.

FIG. 4 is a flow chart illustrating an embodiment of the producing step in FIG. 1.

FIG. 5 is a flow chart illustrating an additional, optional step of the method shown in FIG. 1.

FIG. 6 illustrates a phylogenetic tree indicating the ancestral proteins A3, A5 and A6 according to the embodiments.

FIG. 7 shows thermal unfolding of spike protein variants in different buffers as measured by nano differential scanning fluorimetry. Freshly purified protein samples of HexaPro and ancestral spike protein variants 5 and 6, corresponding to SEQ ID NO: 44 (HexaPro), 12 (A5) and 22 (A6), were diluted to 1.90-2.05 mg/ml in the reference buffer (20 mM HEPES (4-(2-hydroxyethyl)-1-piperazineethanesulfonic acid) pH 7.5, 200 mM NaCl) (panel 1) or the reference buffer containing either 2 M final concentration of urea (panel 2) or guanidine hydrochloride (GDNHCL, panel 3). Protein solutions were soaked into glass capillaries and protein thermal unfolding was measured on a Prometheus NT.48 (NanoTemper) in a temperature range of 20° C.-90° C. with a temperature gradient of 1° C./min. Unfolding was monitored by plotting the first derivative of the 330/350 nm ratio. The obtained values for each protein were normalized to the maximum absolute value obtained with the respective protein variant in any condition and plotted per condition.

FIG. 8 shows shelf-life test of spike protein variants at 4° C. and room temperature. Freshly purified protein samples of HexaPro and ancestral spike protein variants 5 and 6, corresponding to SEQ ID NO: 44 (HexaPro), 12 (A5) and 22 (A6), were diluted to a concentration of ca. 1 mg/ml and stored in a cold room (4° C.) and on the bench (room temperature) for a duration of 3 weeks. At days 0, 3, 7, 14 and 21 the samples were gently mixed by pipetting and 10 μl were transferred to a fresh tube, centrifuged at maximum speed in a tabletop centrifuge for 20 minutes (centrifugation at 4° C. for samples stored in the cold room and at room temperature for samples stored at room temperature) to remove aggregates. 6.5 μl of supernatant were taken from the surface and the concentration of protein in this soluble fraction was determined spectrophotometrically. For better comparability, all concentration values were normalized to the respective concentration measured for each protein at the beginning of the experiment. Samples were incubated in triplicates (three tubes of protein sample stored at the respective temperature) and each replicate data point is indicated by a different symbol. Each individual sample was measured spectrophotometrically in triplicates and technical variation for each data point is indicated by error bars. The average of the three samples is plotted as straight line.

FIG. 9 schematically compares expression yields between HexaPro (H6P) and ancestral spike protein variants 3, 5 and 6, corresponding to SEQ ID NO: 44 (H6P), 2 (A3), 12 (A5) and 22 (A6).

FIG. 10 shows receptor-binding data using surface plasmon resonance (SPR) of HexaPro (H6P) and various ancestral spike protein variants, corresponding to SEQ ID NO: 44 (HexaPro), 2 (A3), 12 (A5), 22 (A6), 19 (A5 wt RBD) and 29 (A6 wt RBD).

FIG. 11 illustrates cryo-EM 3D reconstruction at 2.71 Å and 2.74 Å of A5 trimer and A6 trimer respectively. The monomers constituting the two trimers are shown in different shades of grey.

### DETAILED DESCRIPTION

The present invention generally relates to ancestral sequence reconstruction, and in particular to the production of ancestral protein sequences suitable as antigens, as vaccine candidates and/or for structural studies.

The recent SARS-CoV-2 pandemic has emphasized the need for production of viral proteins that can be used as antigens and in the generation of vaccines. The production should preferably lead to high titers of the viral proteins and the viral protein as such should have sufficient stability to be effectively used as antigen candidate for vaccine production.

This need is not limited to viral proteins but also applies to other pathogens and their pathogen proteins.

The present invention utilizes ancestral sequence reconstruction as starting point for engineering proteins that could be used, among others, as antigen candidates and for vaccine production. Ancestral sequence reconstruction uses the vast and ever-increasing amount of sequence data available in sequence databases to create an alignment of present-day amino acid sequences of a protein family of interest. Phylogenetic and statistical analyses under appropriate models of evolution are then used to define amino acid sequences at the branch points, also referred to as nodes, of the phylogenetic tree generated in the ancestral sequence reconstruction. The so-obtained amino acid sequences at the tree nodes are candidates for ancestral amino acid sequences of the given protein, which have, due to evolution and mutation, given rise to the amino acid sequences of the protein that exists today.

The proteins resulting from production of the ancestral sequences reconstructed according to the invention are found to be robust, yet flexible enough to be used as antigens, vaccine candidates and/or for conducting structural studies. Accordingly, these ancestral sequences adopting robust folds possess several advantages as compared to their modern counterparts. In particular, the ancestral sequences benefit from an inherent robustness making them highly evolvable in allowing further mutations. Furthermore, the robust folds of the ancestral sequences could encompass different binding specificities that are not found in the existing protein versions.

This approach of ancestral sequence reconstruction has been applied to the spike protein (S protein) of coronaviruses, in particular SARS-CoV-2. The spike protein is a key target for antibody binding and vaccine development. However, a major problem with the spike protein is its low stability and low titers upon protein expression. As a consequence, a lot of efforts have been put into structural-guided single amino acid substitutions to stabilize the spike protein. An example of such a comparatively more stable version of the spike protein is the so-called HexaPro construct comprising six proline mutations as compared to the wild type sequence (Hsieh 2020). The spike proteins of the present invention obtained utilizing ancestral sequence reconstruction have similar favorable properties as compared to the HexaPro construct in terms of expression levels and stability. The method of production of the ancestral spike protein constructs is advantageous in that these stable spike protein sequences are generated without the prior need for structural or functional knowledge of the spike protein. Moreover, only few sequences need to be tested (<10 constructs) in order to achieve similar favorable levels of expression and stability as resulting from several rounds of testing and combining individual rational amino acid substitutions by which the HexaPro construct was derived.

Coronaviruses (CoV) constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. Six species of human coronaviruses are known, with one species subdivided into two different strains, making seven strains of human coronaviruses altogether. Four of these strains generally produce mild symptoms of the common cold; human coronavirus OC43 (HCoV-OC43), of the genus β-CoV, human coronavirus HKU1 (HCoV-HKU1), of the genus β-CoV, human coronavirus 229E (HCoV-229E), of the genus α-CoV and human coronavirus NL63 (HCoV-NL63), of the genus α-CoV. Three strains produce symptoms that are potentially severe; all three of these are β-CoV strains; Middle East respiratory syndrome-related coronavirus (MERS-CoV), SARS-CoV, also referred to as SARS-CoV-1, and SARS-CoV-2.

Coronavirus spike protein as used herein refers to a spike protein (S protein) of a coronavirus, preferably a coronavirus selected from the group consisting of MERS-CoV, SARS-CoV and SARS-CoV-2, and more preferably SARS-CoV-2.

An aspect of the present invention relates to a coronavirus spike protein comprising an amino acid sequence according to the formula Seq1-RBD-Seq2. According to the invention, Seq1 comprises an amino acid sequence selected from the group consisting of SEQ ID NO: 5, 15, 25 and an amino acid sequence having at least 90% sequence identity, preferably at least 95% sequence identity, and more preferably at least 97% sequence identity to any of SEQ ID NO: 5, 15 and 25. RBD represents a receptor binding domain and Seq2 comprises an amino acid sequence selected from the group consisting of SEQ ID NO: 8, 18, 28 and an amino acid sequence having at least 90% sequence identity, preferably at least 95% sequence identity, and more preferably at least 97% sequence identity to any of SEQ ID NO: 8, 18 and 28.

As used herein, “sequence identity” refers to sequence similarity between two amino acid sequences (peptide, polypeptide or protein sequences). The similarity is determined by sequence alignment to determine the structural and/or functional relationships between the two sequences.

Sequence identity between amino acid sequences can be determined by comparing an alignment of the sequences using the Needleman-Wunsch Global Sequence Alignment Tool (Needleman and Wunsch 1970) available from the National Center for Biotechnology Information (NCBI), Bethesda, Md., USA, for example via http://blast.ncbi.nlm.nih.gov/Blast.cgi, using default parameter settings (for protein alignment, Gap costs Existence: 11 Extension: 1). When comparing the level of sequence identity to, for example, SEQ ID NO: 5, this should preferably be done relative to the whole length of SEQ ID NO: 5, i.e., a global alignment method is used, to avoid short regions of high identity overlap resulting in a high overall assessment of identity. For example, a short polypeptide fragment having, for example, five amino acids might have a 100% identical sequence to a five amino acid region within the whole of SEQ ID NO: 5, but this does not provide a 100% amino acid identity unless the fragment forms part of a longer sequence, which also has identical amino acids at other positions equivalent to positions in SEQ ID NO: 5. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties. Sequence similarity between amino acid sequences can be determined using a sequence similarity scoring matrix, such as BLOSUM62. Two amino acid sequences have a sequence similarity that is higher than the corresponding sequence identity, for instance by substituting amino acid positions with conservative amino acid substitutions.

An amino acid sequence having at least a defined minimum sequence identity of an amino acid sequence according to a SEQ ID NO: Z, for some value of Z, is preferably obtained by conservative amino acid substitutions in the amino acid sequence according to SEQ ID NO: Z, wherein an amino acid is replaced with a different amino acid with broadly similar properties. Non-conservative substitutions are where amino acids are replaced with amino acids of a different type. By “conservative substitution” is meant the substitution of an amino acid by another amino acid of the same class, in which the classes are defined as follows:
Nonpolar amino acids: A, V, L, I, P, M, F, W
Uncharged polar amino acids: G, S, T, C, Y, N, Q
Acidic amino acids: D, E
Basic amino acids: K, R, H.

As is well known to those skilled in the art, altering the primary structure of a protein by a conservative substitution will not significantly alter the activity or structure of that protein because the side-chain of the amino acid, which is inserted into the amino acid sequence may be able to form similar bonds and contacts as the side chain of the amino acid which has been substituted out. This is so even when the substitution is in a region, which is critical in determining the conformation of the protein.

Seq1 of the coronavirus spike protein is present N-terminally of the receptor binding domain, whereas Seq2 is present C-terminally of the receptor binding domain. The amino acid sequences of Seq1 and Seq2 according to SEQ ID NO: 5, 8, 15, 18, 25 and 28 have been obtained by ancestral sequence reconstruction using the wild-type SARS CoV-2 spike protein (SEQ ID NO: 1) as starting amino acid sequence.

According to the invention, the N-terminal Seq1 comprises an amino acid sequence selected from the group consisting of SEQ ID NO: 5, 15, 25 and an amino acid sequence having at least 90% sequence identity to any of SEQ ID NO: 5, 15 and 25.

In an embodiment, Seq1 comprises an amino acid sequence selected from the group consisting of SEQ ID NO: 5, 15, 25 and an amino acid sequence having at least 95% sequence identity to any of SEQ ID NO: 5, 15 and 25. In preferred embodiment, Seq1 comprises an amino acid sequence selected from the group consisting of SEQ ID NO: 5, 15, 25 and an amino acid sequence having at least 97% sequence identity to any of SEQ ID NO: 5, 15 and 25. In particular embodiments, Seq1 comprises an amino acid sequence selected from the group consisting of SEQ ID NO: 5, 15, 25 and an amino acid sequence having at least 98% or at least 99% sequence identity to any of SEQ ID NO: 5, 15 and 25.

According to an embodiment, Seq1 comprises an amino acid sequence selected from the group consisting of SEQ ID NO: 5, 15 and 25.

According to the invention, the C-terminal Seq2 comprises an amino acid sequence selected from the group consisting of SEQ ID NO: 8, 18, 28 and an amino acid sequence having at least 90% sequence identity to any of SEQ ID NO: 8, 18 and 28.

In an embodiment, Seq2 comprises an amino acid sequence selected from the group consisting of SEQ ID NO: 8, 18, 28 and an amino acid sequence having at least 95% sequence identity to any of SEQ ID NO: 8, 18 and 28. In preferred embodiment, Seq2 comprises an amino acid sequence selected from the group consisting of SEQ ID NO: 8, 18, 28 and an amino acid sequence having at least 97% sequence identity to any of SEQ ID NO: 8, 18 and 28. In particular embodiments, Seq2 comprises an amino acid sequence selected from the group consisting of SEQ ID NO: 8, 18, 28 and an amino acid sequence having at least 98% or at least 99% sequence identity to any of SEQ ID NO: 8, 18 and 28.

According to an embodiment, Seq2 comprises an amino acid sequence selected from the group consisting of SEQ ID NO: 8, 18 and 28.

The receptor binding domain of the coronavirus spike protein could be any receptor binding domain, and in particular any receptor binding domain of a coronavirus spike protein. In a preferred embodiment, the receptor binding domain of the coronavirus spike protein is a receptor binding domain of a spike protein of a coronavirus selected from the group consisting of MERS-CoV, SARS-CoV and SARS-CoV-2, and more preferably SARS-CoV-2.

In an embodiment, the receptor binding domain comprises, such as consists of, an amino acid sequence selected from the group consisting of SEQ ID NO: 7, 17, 27, 32 and an amino acid sequence having at least 90% sequence identity to any of SEQ ID NO: 7, 17, 27 and 32. In a preferred embodiment, the receptor binding domain comprises, such as consists of, an amino acid sequence selected from the group consisting of SEQ ID NO: 7, 17, 27, 32 and an amino acid sequence having at least 95% sequence identity to any of SEQ ID NO: 7, 17, 27 and 32. In a more preferred embodiment, the receptor binding domain comprises, such as consists of, an amino acid sequence selected from the group consisting of SEQ ID NO: 7, 17, 27, 32 and an amino acid sequence having at least 97% sequence identity to any of SEQ ID NO: 7, 17, 27 and 32. In particular embodiments, the receptor binding domain comprises, such as consists of, an amino acid sequence selected from the group consisting of SEQ ID NO: 7, 17, 27, 32 and an amino acid sequence having at least 98% or at least 99% sequence identity to any of SEQ ID NO: 7, 17, 27 and 32.

According to an embodiment, the receptor binding domain comprises, such as consists of, an amino acid sequence selected from the group consisting of SEQ ID NO: 7, 17, 27 and 32.

The receptor binding domains as defined in SEQ ID NO: 7, 17 and 27 have been obtained by ancestral sequence reconstruction using the wild-type SARS CoV-2 spike protein (SEQ ID NO: 1) as starting amino acid sequence. The receptor binding domain as defined in SEQ ID NO: 32 is the receptor binding domain of the wild-type SARS Cov-2 spike protein (SEQ ID NO: 1).

In an embodiment, the receptor binding domain comprises, such as consists of, an amino acid sequence selected from the group consisting of SEQ ID NO: 7, 17, 27.

In an embodiment, the receptor binding domain comprises, such as consists of, an amino acid sequence of SEQ ID NO: 32.

Seq1 may comprise other amino acid sequences in addition to the amino acid sequence selected from the group consisting of SEQ ID NO: 5, 15, 25 and an amino acid sequence having at least 90% sequence identity to any of SEQ ID NO: 5, 15 and 25. An example of such another amino acid sequence that could be present in Seq1 is an N-terminal signal peptide, sometimes referred to as signal sequence, targeting sequence, localization signal, localization sequence, transit peptide, leader sequence or a leader peptide. Such an N-terminal signal peptide is a short peptide present at the N-terminus of most newly synthesized proteins that are destined toward the secretory pathway. These proteins include those that reside either inside certain organelles, such as the endoplasmic reticulum, Golgi or endosomes, secreted from the cell, or inserted into most cellular membranes.

In an embodiment, Seq1 comprises, such as consists of, an N-terminal signal peptide and the amino acid sequence selected from the group consisting of SEQ ID NO: 5, 15, 25 and an amino acid sequence having at least 90% sequence identity to any of SEQ ID NO: 5, 15 and 25. An illustrative, but non-limiting, example of an N-terminal signal peptide that could be used according to the present invention is an N-terminal signal peptide of the wild-type SARS CoV-2 spike protein (SEQ ID NO: 1). Such an N-terminal signal peptide is MFVFLVLLPLVSS as defined in SEQ ID NO: 33.

In an embodiment, Seq1 comprises, such as consists of, an amino acid sequence selected from the group consisting of SEQ ID NO: 6, 16, 26 and an amino acid sequence having at least 90% sequence identity, preferably at least 95% sequence identity, and more preferably at least 97% sequence identity to any of SEQ ID NO: 6, 16 and 26. In particular embodiments, Seq1 comprises, such as consists of, an amino acid sequence selected from the group consisting of SEQ ID NO: 6, 16, 26 and an amino acid sequence having at least 98% or at least 99% sequence identity to any of SEQ ID NO: 6, 16 and 26.

According to an embodiment, Seq1 comprises, such as consists of, an amino acid sequence selected from the group consisting of SEQ ID NO: 6, 16 and 26.

SEQ ID NO: 6, 16 and 26 correspond to the amino acid sequences of SEQ ID NO: 5, 15 and 25 preceded by the N-terminal signal peptide according to SEQ ID NO: 33.

In an embodiment, Seq1 comprises an amino acid sequence according to SEQ ID NO: 25 or an amino acid sequence having at least 90% sequence identity, preferably at least 95% sequence identity, and more preferably at least 97% sequence identity to SEQ ID NO: 25. In this embodiment, Seq2 comprises an amino acid sequence according to SEQ ID NO: 28 or an amino acid sequence having at least 90% sequence identity, preferably at least 95% sequence identity, and more preferably at least 97% sequence identity to SEQ ID NO: 28. In particular embodiments, Seq1 comprises an amino acid sequence according to SEQ ID NO: 25 or an amino acid sequence having at least 98% or 99% sequence identity to SEQ ID NO: 25 and Seq2 comprises an amino acid sequence according to SEQ ID NO: 28 or an amino acid sequence having at least 98% or 99% sequence identity to SEQ ID NO: 28. For instance, Seq1 comprises an amino acid sequence according to SEQ ID NO: 25 and Seq2 comprises an amino acid sequence according to SEQ ID NO: 28.

In a particular embodiment, Seq1 comprises, such as consists of, an amino acid sequence according to SEQ ID NO: 26 or an amino acid sequence having at least 90% sequence identity, preferably at least 95% sequence identity, and more preferably at least 97% sequence identity to SEQ ID NO: 26. In particular embodiments, Seq1 comprises, such as consists of, an amino acid sequence according to SEQ ID NO: 26 or an amino acid sequence having at least 98% or 99% sequence identity to SEQ ID NO: 26. Preferably, Seq1 comprises, such as consists of, the amino acid sequence according to SEQ ID NO: 26.

In a particular embodiment, the receptor binding domain comprises, such as consists of, an amino acid sequence selected from the group consisting of SEQ ID NO: 27, 32 and an amino acid sequence having at least 90% sequence identity, preferably at least 95% sequence identity, and more preferably at least 97% sequence identity to any of SEQ ID NO: 27 and 32. For instance, the receptor binding domain comprises, such as consists of, an amino acid sequence selected from the group consisting of SEQ ID NO: 27, 32 and an amino acid sequence having at least 98% or 99% sequence identity to any of SEQ ID NO: 27 and 32. Preferably, the receptor binding domain comprises, such as consists of, an amino acid sequence selected from the group consisting of SEQ ID NO: 27 and 32.

In an embodiment, the receptor binding domain comprises, such as consists of, an amino acid sequence selected from the group consisting of SEQ ID NO: 27 and an amino acid sequence having at least 90% sequence identity, preferably at least 95% sequence identity, and more preferably at least 97% sequence identity to any of SEQ ID NO: 27. For instance, the receptor binding domain comprises, such as consists of, an amino acid sequence selected from the group consisting of SEQ ID NO: 27 and an amino acid sequence having at least 98% or 99% sequence identity to SEQ ID NO: 27. Preferably, the receptor binding domain comprises, such as consists of, an amino acid sequence of SEQ ID NO: 27.

In an embodiment, the receptor binding domain comprises, such as consists of, an amino acid sequence selected from the group consisting of SEQ ID NO: 32 and an amino acid sequence having at least 90% sequence identity, preferably at least 95% sequence identity, and more preferably at least 97% sequence identity to any of SEQ ID NO: 32. For instance, the receptor binding domain comprises, such as consists of, an amino acid sequence selected from the group consisting of SEQ ID NO: 32 and an amino acid sequence having at least 98% or 99% sequence identity to SEQ ID NO: 32. Preferably, the receptor binding domain comprises, such as consists of, an amino acid sequence of SEQ ID NO: 32.

An example of a coronavirus spike protein according to the invention comprises, such as consists of, an amino acid sequence selected from the group consisting of SEQ ID NO: 22, 23, 24, 29, 30, 31 and an amino acid sequence having at least 90% sequence identity, preferably at least 95% sequence identity, and more preferably at least 97% sequence identity to any of SEQ ID NO: 22, 23, 24, 29, 30 and 31. In a particular embodiment, the coronavirus spike protein comprises, such as consists of, an amino acid sequence selected from the group consisting of SEQ ID NO: 22, 23, 24, 29, 30, 31 and an amino acid sequence having at least 98% or 99% sequence identity to any of SEQ ID NO: 22, 23, 24, 29, 30 and 31. In a particular embodiment, the coronavirus spike protein comprises, such as consists of, an amino acid sequence selected from the group consisting of SEQ ID NO: 22, 23, 24, 29, 30 and 31.

The coronavirus spike protein as defined in SEQ ID NO: 22 has been obtained by ancestral sequence reconstruction according to the invention. This spike protein comprises an N-terminal signal peptide (SEQ ID NO: 33) and additional C-terminal sequences (SEQ ID NO: 34, 35, 36 and 38). These additional C-terminal sequences include a GS-linker (GS), a T4-FoldOn trimerization domain (GYIPEAPRDGQAYVRKDGEWVLLSTFL, SEQ ID NO: 34), a GTS-linker (GTS), a human rhinovirus (HRV) 3C protease restriction site (LEVLFQGP, SEQ ID NO: 35), a G-linker (G), a His₈-tag (HHHHHHHH, SEQ ID NO: 36) and a Twin-Strep-Tag® (SAWSHPQFEKGGGSGGGGSGGSAWSHPQFEK, SEQ ID NO: 37).

The T4-FoldOn trimerization domain corresponds to the C-terminal domain of T4 fibritin and facilitates formation of a homotrimeric structure. The His₈-tag and Twin Strep-Tag® facilitate purification of the spike protein, whereas the HRV 3C protease restriction site enables removal of the purification tags (His₈-tag and Twin Strep-Tag®) by HRV 3C protease treatment. The GS-, GTS- and G-linkers are included to provide flexible linkers between the C-terminal subdomains.

The coronavirus spike protein as defined in SEQ ID NO: 23 has been obtained by ancestral sequence reconstruction according to the invention. This spike protein comprises an N-terminal signal peptide (SEQ ID NO: 33) but lacks the C-terminal sequences of SEQ ID NO: 22.

The coronavirus spike protein as defined in SEQ ID NO: 24 has been obtained by ancestral sequence reconstruction according to the invention. This spike protein lacks the N-terminal signal peptide of SEQ ID NO: 22 and 23 and lacks the C-terminal sequences of SEQ ID NO: 22.

The coronavirus spike protein as defined in SEQ ID NO: 29 has been obtained by ancestral sequence reconstruction according to the invention. This spike protein comprises an N-terminal signal peptide (SEQ ID NO: 33) and the additional C-terminal sequences (SEQ ID NO: 34, 35, 36 and 38). This spike protein furthermore has a receptor binding domain of wild-type SARS-CoV-2 (SEQ ID NO: 1) with the wild-type receptor binding domain as defined in SEQ ID NO: 32.

The coronavirus spike protein as defined in SEQ ID NO: 30 has been obtained by ancestral sequence reconstruction according to the invention. This spike protein comprises an N-terminal signal peptide (SEQ ID NO: 33) but lacks the C-terminal sequences of SEQ ID NO: 29. This spike protein furthermore has a receptor binding domain of wild-type SARS-CoV-2 (SEQ ID NO: 1) with the wild-type receptor binding domain as defined in SEQ ID NO: 32.

The coronavirus spike protein as defined in SEQ ID NO: 31 has been obtained by ancestral sequence reconstruction according to the invention. This spike protein lacks the N-terminal signal peptide of SEQ ID NO: 29 and 30 and lacks the C-terminal sequences of SEQ ID NO: 29. The spike protein furthermore has a receptor binding domain of wild-type SARS-CoV-2 (SEQ ID NO: 1) with the wild-type receptor binding domain as defined in SEQ ID NO: 32.

In another embodiment, Seq1 comprises an amino acid sequence according to SEQ ID NO: 5 or an amino acid sequence having at least 90% sequence identity, preferably at least 95% sequence identity, and more preferably at least 97% sequence identity to SEQ ID NO: 5. In this embodiment, Seq2 comprises an amino acid sequence according to SEQ ID NO: 8 or an amino acid sequence having at least 90% sequence identity, preferably at least 95% sequence identity, and more preferably at least 97% sequence identity to SEQ ID NO: 8. In particular embodiments, Seq1 comprises an amino acid sequence according to SEQ ID NO: 5 or an amino acid sequence having at least 98% or 99% sequence identity to SEQ ID NO: 5 and Seq2 comprises an amino acid sequence according to SEQ ID NO: 8 or an amino acid sequence having at least 98% or 99% sequence identity to SEQ ID NO: 8. For instance, Seq1 comprises an amino acid sequence according to SEQ ID NO: 5 and Seq2 comprises an amino acid sequence according to SEQ ID NO: 8.

In a particular embodiment, Seq1 comprises, such as consists of, an amino acid sequence according to SEQ ID NO: 6 or an amino acid sequence having at least 90% sequence identity, preferably at least 95% sequence identity, and more preferably at least 97% sequence identity to SEQ ID NO: 6. In particular embodiments, Seq1 comprises, such as consists of, an amino acid sequence according to SEQ ID NO: 6 or an amino acid sequence having at least 98% or 99% sequence identity to SEQ ID NO: 6. Preferably, Seq1 comprises, such as consists of, the amino acid sequence according to SEQ ID NO: 6.

In a particular embodiment, the receptor binding domain comprises, such as consists of, an amino acid sequence selected from the group consisting of SEQ ID NO: 7, 32 and an amino acid sequence having at least 90% sequence identity, preferably at least 95% sequence identity, and more preferably at least 97% sequence identity to any of SEQ ID NO: 7 and 32. For instance, the receptor binding domain comprises, such as consists of, an amino acid sequence selected from the group consisting of SEQ ID NO: 7, 32 and an amino acid sequence having at least 98% or 99% sequence identity to any of SEQ ID NO: 7 and 32. Preferably, the receptor binding domain comprises, such as consists of, an amino acid sequence selected from the group consisting of SEQ ID NO: 7 and 32.

In an embodiment, the receptor binding domain comprises, such as consists of, an amino acid sequence selected from the group consisting of SEQ ID NO: 7 and an amino acid sequence having at least 90% sequence identity, preferably at least 95% sequence identity, and more preferably at least 97% sequence identity to any of SEQ ID NO: 7. For instance, the receptor binding domain comprises, such as consists of, an amino acid sequence selected from the group consisting of SEQ ID NO: 7 and an amino acid sequence having at least 98% or 99% sequence identity to SEQ ID NO: 7. Preferably, the receptor binding domain comprises, such as consists of, an amino acid sequence of SEQ ID NO: 7.

In an embodiment, the receptor binding domain comprises, such as consists of, an amino acid sequence selected from the group consisting of SEQ ID NO: 32 and an amino acid sequence having at least 90% sequence identity, preferably at least 95% sequence identity, and more preferably at least 97% sequence identity to any of SEQ ID NO: 32. For instance, the receptor binding domain comprises, such as consists of, an amino acid sequence selected from the group consisting of SEQ ID NO: 32 and an amino acid sequence having at least 98% or 99% sequence identity to SEQ ID NO: 32. Preferably, the receptor binding domain comprises, such as consists of, an amino acid sequence of SEQ ID NO: 32.

An example of a coronavirus spike protein according to the invention comprises, such as consists of, an amino acid sequence selected from the group consisting of SEQ ID NO: 2, 3, 4, 9, 10, 11 and an amino acid sequence having at least 90% sequence identity, preferably at least 95% sequence identity, and more preferably at least 97% sequence identity to any of SEQ ID NO: 2, 3, 4, 9, 10 and 11. In a particular embodiment, the coronavirus spike protein comprises, such as consists of, an amino acid sequence selected from the group consisting of SEQ ID NO: 2, 3, 4, 9, 10, 11 and an amino acid sequence having at least 98% or 99% sequence identity to any of SEQ ID NO: 2, 3, 4, 9, 10 and 11. In a particular embodiment, the coronavirus spike protein comprises, such as consists of, an amino acid sequence selected from the group consisting of SEQ ID NO: 2, 3, 4, 9, 10 and 11.

The coronavirus spike protein as defined in SEQ ID NO: 2 has been obtained by ancestral sequence reconstruction according to the invention. This spike protein comprises an N-terminal signal peptide (SEQ ID NO: 33) and the additional C-terminal sequences (SEQ ID NO: 34, 35, 36 and 38).

The coronavirus spike protein as defined in SEQ ID NO: 3 has been obtained by ancestral sequence reconstruction according to the invention. This spike protein comprises an N-terminal signal peptide (SEQ ID NO: 33) but lacks the C-terminal sequences of SEQ ID NO: 2.

The coronavirus spike protein as defined in SEQ ID NO: 4 has been obtained by ancestral sequence reconstruction according to the invention. This spike protein lacks the N-terminal signal peptide of SEQ ID NO: 2 and 3 and lacks the C-terminal sequences of SEQ ID NO: 2.

The coronavirus spike protein as defined in SEQ ID NO: 9 has been obtained by ancestral sequence reconstruction according to the invention. This spike protein comprises an N-terminal signal peptide (SEQ ID NO: 33) and the additional C-terminal sequences (SEQ ID NO: 34, 35, 36 and 38). This spike protein furthermore has a receptor binding domain of wild-type SARS-CoV-2 (SEQ ID NO: 1) with the wild-type receptor binding domain as defined in SEQ ID NO: 32.

The coronavirus spike protein as defined in SEQ ID NO: 10 has been obtained by ancestral sequence reconstruction according to the invention. This spike protein comprises an N-terminal signal peptide (SEQ ID NO: 33) but lacks the C-terminal sequences of SEQ ID NO: 9. This spike protein furthermore has a receptor binding domain of wild-type SARS-CoV-2 (SEQ ID NO: 1) with the wild-type receptor binding domain as defined in SEQ ID NO: 32.

The coronavirus spike protein as defined in SEQ ID NO: 11 has been obtained by ancestral sequence reconstruction according to the invention. This spike protein lacks the N-terminal signal peptide of SEQ ID NO: 9 and 10 and lacks the C-terminal sequences of SEQ ID NO: 9. The spike protein furthermore has a receptor binding domain of wild-type SARS-CoV-2 (SEQ ID NO: 1) with the wild-type receptor binding domain as defined in SEQ ID NO: 32.

In a further embodiment, Seq1 comprises an amino acid sequence according to SEQ ID NO: 15 or an amino acid sequence having at least 90% sequence identity, preferably at least 95% sequence identity, and more preferably at least 97% sequence identity to SEQ ID NO: 15. In this embodiment, Seq2 comprises an amino acid sequence according to SEQ ID NO: 18 or an amino acid sequence having at least 90% sequence identity, preferably at least 95% sequence identity, and more preferably at least 97% sequence identity to SEQ ID NO: 18. In particular embodiments, Seq1 comprises an amino acid sequence according to SEQ ID NO: 15 or an amino acid sequence having at least 98% or 99% sequence identity to SEQ ID NO: 15 and Seq2 comprises an amino acid sequence according to SEQ ID NO: 18 or an amino acid sequence having at least 98% or 99% sequence identity to SEQ ID NO: 18. For instance, Seq1 comprises an amino acid sequence according to SEQ ID NO: 15 and Seq2 comprises an amino acid sequence according to SEQ ID NO: 18.

In a particular embodiment, Seq1 comprises, such as consists of, an amino acid sequence according to SEQ ID NO: 16 or an amino acid sequence having at least 90% sequence identity, preferably at least 95% sequence identity, and more preferably at least 97% sequence identity to SEQ ID NO: 16. In particular embodiments, Seq1 comprises, such as consists of, an amino acid sequence according to SEQ ID NO: 16 or an amino acid sequence having at least 98% or 99% sequence identity to SEQ ID NO: 16. Preferably, Seq1 comprises, such as consists of, the amino acid sequence according to SEQ ID NO: 16.

In a particular embodiment, the receptor binding domain comprises, such as consists of, an amino acid sequence selected from the group consisting of SEQ ID NO: 17, 32 and an amino acid sequence having at least 90% sequence identity, preferably at least 95% sequence identity, and more preferably at least 97% sequence identity to any of SEQ ID NO: 17 and 32. For instance, the receptor binding domain comprises, such as consists of, an amino acid sequence selected from the group consisting of SEQ ID NO: 17, 32 and an amino acid sequence having at least 98% or 99% sequence identity to any of SEQ ID NO: 17 and 32. Preferably, the receptor binding domain comprises, such as consists of, an amino acid sequence selected from the group consisting of SEQ ID NO: 17 and 32.

In an embodiment, the receptor binding domain comprises, such as consists of, an amino acid sequence selected from the group consisting of SEQ ID NO: 17 and an amino acid sequence having at least 90% sequence identity, preferably at least 95% sequence identity, and more preferably at least 97% sequence identity to any of SEQ ID NO: 17. For instance, the receptor binding domain comprises, such as consists of, an amino acid sequence selected from the group consisting of SEQ ID NO: 17 and an amino acid sequence having at least 98% or 99% sequence identity to SEQ ID NO: 17. Preferably, the receptor binding domain comprises, such as consists of, an amino acid sequence of SEQ ID NO: 17.

In an embodiment, the receptor binding domain comprises, such as consists of, an amino acid sequence selected from the group consisting of SEQ ID NO: 32 and an amino acid sequence having at least 90% sequence identity, preferably at least 95% sequence identity, and more preferably at least 97% sequence identity to any of SEQ ID NO: 32. For instance, the receptor binding domain comprises, such as consists of, an amino acid sequence selected from the group consisting of SEQ ID NO: 32 and an amino acid sequence having at least 98% or 99% sequence identity to SEQ ID NO: 32. Preferably, the receptor binding domain comprises, such as consists of, an amino acid sequence of SEQ ID NO: 32.

An example of a coronavirus spike protein according to the invention comprises, such as consists of, an amino acid sequence selected from the group consisting of SEQ ID NO: 12, 13, 14, 19, 20, 21 and an amino acid sequence having at least 90% sequence identity, preferably at least 95% sequence identity, and more preferably at least 97% sequence identity to any of SEQ ID NO: 12, 13, 14, 19, 20 and 21. In a particular embodiment, the coronavirus spike protein comprises, such as consists of, an amino acid sequence selected from the group consisting of SEQ ID NO: 12, 13, 14, 19, 20, 21 and an amino acid sequence having at least 98% or 99% sequence identity to any of SEQ ID NO: 12, 13, 14, 19, 20 and 21. In a particular embodiment, the coronavirus spike protein comprises, such as consists of, an amino acid sequence selected from the group consisting of SEQ ID NO: 12, 13, 14, 19, 20 and 21.

The coronavirus spike protein as defined in SEQ ID NO: 12 has been obtained by ancestral sequence reconstruction according to the invention. This spike protein comprises an N-terminal signal peptide (SEQ ID NO: 33) and the additional C-terminal sequences (SEQ ID NO: 34, 35, 36 and 38).

The coronavirus spike protein as defined in SEQ ID NO: 13 has been obtained by ancestral sequence reconstruction according to the invention. This spike protein comprises an N-terminal signal peptide (SEQ ID NO: 33) but lacks the C-terminal sequences of SEQ ID NO: 12.

The coronavirus spike protein as defined in SEQ ID NO: 14 has been obtained by ancestral sequence reconstruction according to the invention. This spike protein lacks the N-terminal signal peptide of SEQ ID NO: 12 and 13 and lacks the C-terminal sequences of SEQ ID NO: 12.

The coronavirus spike protein as defined in SEQ ID NO: 19 has been obtained by ancestral sequence reconstruction according to the invention. This spike protein comprises an N-terminal signal peptide (SEQ ID NO: 33) and the additional C-terminal sequences (SEQ ID NO: 34, 35, 36 and 38). This spike protein furthermore has a receptor binding domain of wild-type SARS-CoV-2 (SEQ ID NO: 1) with the wild-type receptor binding domain as defined in SEQ ID NO: 32.

The coronavirus spike protein as defined in SEQ ID NO: 20 has been obtained by ancestral sequence reconstruction according to the invention. This spike protein comprises an N-terminal signal peptide (SEQ ID NO: 33) but lacks the C-terminal sequences of SEQ ID NO: 19. This spike protein furthermore has a receptor binding domain of wild-type SARS-CoV-2 (SEQ ID NO: 1) with the wild-type receptor binding domain as defined in SEQ ID NO: 32.

The coronavirus spike protein as defined in SEQ ID NO: 21 has been obtained by ancestral sequence reconstruction according to the invention. This spike protein lacks the N-terminal signal peptide of SEQ ID NO: 19 and 20 and lacks the C-terminal sequences of SEQ ID NO: 19. The spike protein furthermore has a receptor binding domain of wild-type SARS-CoV-2 (SEQ ID NO: 1) with the wild-type receptor binding domain as defined in SEQ ID NO: 32.

In an embodiment, the coronavirus spike protein comprises, preferably consists of, an amino acid sequence selected from the group consisting of SEQ ID NO: 2, 3, 4, 9, 10, 11, 12, 13, 14, 19, 20, 21, 22, 23, 24, 29, 30, 31 and an amino acid sequence having at least 90% sequence identity, preferably at least 95% sequence identity, and more preferably at least 97% sequence identity to any of SEQ ID NO: 2, 3, 4, 9, 10, 11, 12, 13, 14, 19, 20, 21, 22, 23, 24, 29, 30, 31.

In a particular embodiment, the coronavirus spike protein comprises, preferably consists of, an amino acid sequence selected from the group consisting of SEQ ID NO: 2, 3, 4, 12, 13, 14, 22, 23, 24, and an amino acid sequence having at least 90% sequence identity, preferably at least 95% sequence identity, and more preferably at least 97% sequence identity to any of SEQ ID NO: 2, 3, 4, 12, 13, 14, 22, 23, 24.

In another particular embodiment, the coronavirus spike protein comprises, preferably consists of, an amino acid sequence selected from the group consisting of SEQ ID NO: 9, 10, 11, 19, 20, 21, 29, 30, 31, and an amino acid sequence having at least 90% sequence identity, preferably at least 95% sequence identity, and more preferably at least 97% sequence identity to any of SEQ ID NO: 9, 10, 11, 19, 20, 21, 29, 30, 31.

The coronavirus spike protein of the invention could consist of the amino acid sequence according to the formula Seq1-RBD-Seq2. In another embodiment, the coronavirus spike protein comprises multiple amino acid sequences according to the formula Seq1-RBD-Seq2. In such another embodiment, the multiple amino acid sequences could all comprise the same Seq1 amino acid sequence or could comprises different Seq1 amino acid sequences according to the invention. Alternatively, or in addition, the multiple amino acid sequences could all comprise the same RBD amino acid sequence or could comprises different RBD amino acid sequences according to the invention. Alternatively, or in addition, the multiple amino acid sequences could all comprise the same Seq2 amino acid sequence or could comprises different Seq2 amino acid sequences according to the invention. For instance, a coronavirus spike protein could comprise, such as consist of, an amino acid sequence according to the formula Seq1₁-RBD₁-Seq2₁-L-Seq1₂-RBD₂-Seq2₂. In such an example, Seq1₁ is the same as Seq1₂ or Seq1₁ is different than Seq1₂, RBD₁ is the same as RBD₂ or RBD₁ is different than RBD₂ and Seq2₁ is the same as Seq2₂ or Seq2₁ is different than Seq2₂. L is an optional linker. In the above-described embodiments, a subset of the RBD amino acid sequences could be an epitope sequence that is different from a receptor binding domain.

In an embodiment, the coronavirus spike protein of the invention is an isolated coronavirus spike protein.

The present invention also relates to a nucleic acid molecule encoding a coronavirus spike protein according to the invention.

Nucleic acid molecule as used herein includes a polynucleotide and nucleic acid sequence, and generally means a polymer of deoxyribonucleic acid (DNA) or ribonucleic acid (RNA), which may be single-stranded or double-stranded, which may contain natural, non-natural or altered nucleotides, and which may contain a natural, non-natural or altered internucleotide linkage, such as a phosphoroamidate linkage or a phosphorothioate linkage, instead of the phosphodiester found between the nucleotides of an unmodified oligonucleotide. Nucleic acid molecule also includes complementary DNA (cDNA) and messenger RNA (mRNA).

Examples of nucleic acid molecule according to the embodiments are shown in SEQ ID NO: 41-43 encoding the coronavirus spike proteins as defined in SEQ ID NO: 9, 19 and 29, respectively. The corresponding nucleic acid molecules encoding the corona spike proteins as defined in SEQ ID NO: 2, 12 and 22 are shown in SEQ ID NO: 38-40.

The nucleic acid molecules presented below have the following general formula: SP-Seq1-RBD-Seq2-GS-T4-GTS-HRV 3C-G-HIS-Twin-Strep-Stop, wherein SP represents a signal peptide, GS represents a GS linker, T4 represents a T4-FoldOn trimerization domain, GTS represents a GTS linker, HRV C3 represents a HRV 3C protease restriction site, G represents a G linker, HIS represents a HIS₈-tag, Twin-Strep represents a Twin-Strep-Tag® and Stop represents a stop codon.

A3-
(SEQ ID NO: 38)
ATGTTCGTGTTTCTGGTGCTGCTGCCTCTGGTGTCCAGCCAGTGTGTGAATCTGACCGGCAG
AACCCCTCTGAACCCCAACTACACCAACAGCAGCCAGCGGGGCGTGTACTACCCCGACACCA
TCTTTAGAAGCGACACCCTGGTGCTGAGCCAGGGCTACTTCCTGCCTTTCTACAGCAACGTG
TCCTGGTACTACAGCCTGACCACCAACAACGCCGCCACCAAGAGATTCGACAACCCCATCCT
GGACTTCAAGGACGGCATCTACTTTGCCGCCACCGAGAAGTCCAACATCATCAGAGGCTGGA
TCTTCGGCACCACACTGGACAACACAAGCCAGAGCCTGCTGATCGTGAACAACGCCACCAAC
GTGATCATCAAAGTGTGCAACTTCCAGTTCTGCTACGACCCCTACCTGAGCGGCTACTACGG
CCACAACAACAAGACCTGGTCCATCCGCGAGTTCGCCGTGTACAGCAGCTACGCCAATTGCA
CCTTCGAGTACGTGTCCAAGAGCTTCATGCTGGACATCAGCGGCAAAGGCGGCCTGTTCAAT
ACCCTGCGCGAGTTTGTGTTCAGAAACGTGGACGGCTACTTCAAGATCTACTCCAAGTACAC
CCCTGTGAACCTGAACCGGGGCCTGCCTACAGGCTTTTCTGTTCTGCAGCCCCTGGTGGAAC
TGCCCGTGGGCATCAACATCACCAAGTTCAGAACCCTGCTGACCATCCACAGAGGCGACCCC
ATGCCTAACAATGGCTGGACCGCCTTTAGCGCCGCCTACTTCGTGGGCTACCTGAAGCCTCG
GACCTTTATGCTGAAGTACAACGAGAACGGCACCATCACCGACGCCGTGGATTGTGCCCTTG
ATCCTCTGAGCGAGACAAAGTGCACCCTGAAGTCCCTGACAGTGCAGAAGGGCATCTACCAG
ACCAGCAACTTCCGGGTGCAGCCCACACAGTCTGTCGTGCGGTTCCCCAATATCACCAATCT
GTGCCCCTTCCACAAGGTGTTCAACGCCACAAGATTCCCCAGCGTGTACGCCTGGGAGCGCA
CCAAGATCTCTGATTGCGTGGCCGACTACACCGTGTTCTACAACAGCACCTCCTTCAGCACC
TTCAAGTGCTACGGCGTGTCCCCTAGCAAGCTGATCGACCTGTGCTTCACCTCTGTGTACGC
CGACACCTTCCTGATCCGGTTCAGCGAAGTGCGACAGGTGGCACCTGGACAGACAGGCGTGA
TCGCCGATTACAACTACAAGCTGCCCGACGACTTCACCGGCTGTGTGATCGCCTGGAATACC
GCCAAGCAGGACGTGGGCAACTACTTCTACAGAAGCCACAGAAGCACCAAGCTGAAGCCCTT
CGAGAGGGACCTGAGCAGCCAGGATGAGAATGGCGTGCGGACCCTGAGCACCTACGACTTCA
ACCCCAATGTGCCCCTGGAATATCAGGCCACCAGAGTGGTGGTGCTGTCCTTCGAGCTGCTG
AATGCCCCTGCCACAGTGTGTGGCCCTAAGCTGTCTACCCAGCTGGTCAAGAACCAGTGCGT
GAACTTCAACTTCAACGGCCTGAAAGGCACCGGCGTGCTGACCGATAGCAGCAAGAGATTTC
AGAGCTTCCAGCAGTTCGGCCGGGACGCCAGCGATTTCACAGACAGCGTTAGAGATCCCCAG
ACACTGGAAATCCTGGATATCACCCCTTGCAGCTTCGGCGGAGTGTCCGTGATCACCCCTGG
CACAAATACCAGCTCTGAGGTGGCCGTGCTGTACCAGGATGTGAACTGCACCGATGTGCCCA
CAGCCATCCATGCCGATCAGCTGACACCCGCCTGGCGGATCTATAGCACCGGCACCAATGTG
TTTCAGACCCAAGCCGGCTGTCTGATTGGAGCCGAGCATGTGAATGCCAGCTACGAGTGCGA
CATCCCTATCGGAGCCGGCATCTGCGCCAGCTACCACACAGCCTCTATCCTGAGATCCACCA
GCCAGAAAGCCATCGTGGCCTACACAATGTCTCTGGGCGCCGAGAACTCTATCGCCTACGCC
AACAACAGCATTGCTATCCCCACCAACTTCTCCATCAGCGTGACCACCGAAGTGATGCCCGT
GTCCATGGCCAAGACCAGCGTGGACTGCACCATGTACATCTGCGGCGACAGCATCGAGTGCA
GCAACCTGCTGCTGCAGTACGGCAGCTTCTGCACCCAGCTGAATAGAGCCCTGACCGGAATC
GCCATCGAGCAGGACAAGAACACCCAAGAGGTGTTCGCCCAAGTGAAGCAGATCTACAAGAC
CCCTCCTATCAAGGACTTCGGCGGCTTCAATTTCAGCCAGATCCTGCCAGATCCAAGCAAGC
CCAGCAAGCGGAGCTTCATCGAGGACCTGCTGTTCAACAAAGTGACACTGGCCGACGCCGGC
TTCATCAAGCAGTATGGCGATTGCCTGGGCGACATCTCCGCCAGGGATCTGATTTGCGCCCA
GAAGTTCAACGGACTGACCGTGCTGCCTCCTCTGCTGACAGATGAGATGATCGCCGCCTATA
CCGCCGCTCTGATCTCTGGAACAGCCACCGCCGGATGGACATTTGGAGCTGGTGCAGCTCTG
CAGATCCCCTTCGCTATGCAGATGGCCTACCGGTTCAATGGCATCGGCGTGACCCAGAATGT
GCTGTACGAGAACCAGAAGCTGATTGCCAACCAGTTCAACAGCGCCATCGGCAAGATCCAAG
AGAGCCTGACCTCTACCGCCAGCGCTCTGGGAAAGCTGCAGGACGTCGTGAACCAGAACGCC
CAGGCTCTGAACACCCTCGTGAAGCAGCTGAGCAGCAATTTCGGCGCCATCAGCAGCGTGCT
GAACGACATCCTGAGCCGGCTGGATAAGGTGGAAGCCGAGGTGCAGATCGACCGGCTGATTA
CAGGCAGACTGCAGAGCCTCCAGACCTACGTGACACAGCAGCTGATCAGAGCCGCCGAGATT
AGAGCCTCTGCCAATCTGGCCGCCACAAAGATGAGCGAGTGTGTGCTGGGCCAGAGCAAGAG
AGTGGACTTTTGCGGCAAGGGCTACCACCTGATGAGCTTCCCTCAGTCTGCTCCTCACGGCG
TGGTGTTTCTGCACGTGACATACGTGCCCAGCCAAGAGAAGAACTTCACCACCGCTCCAGCC
ATCTGCCACGAGGGCAAAGCCCACTTTCCTAGAGAAGGCGTGTTCGTGTCCAACGGCACCCA
TTGGTTCGTGACTCAGCGGAACTTCTACGAGCCCCAGATCATCACCACCGACAACACCTTCG
TGTCCGGCAACTGCGACGTGGTCATCGGCATCATCAACAATACCGTGTACGACCCACTGCAG
CCCGAGCTGGACAGCTTCAAAGAGGAACTGGACAAGTACTTTAAGAACCACACAAGCCCCGA
CGTGGACCTGGGCGATATCAGCGGAATCAATGCCAGCGTGGTCAACATCCAGAAAGAGATCG
ATAGGCTGAACGAGGTGGCCAAGAACCTGAATGAGTCCCTGATTGACCTGCAAGAGCTGGGG
AAGTACGAGCAAGGATCTGGCTATATTCCCGAGGCTCCTAGAGATGGCCAGGCCTACGTTAG
AAAGGACGGCGAGTGGGTCCTGCTGAGCACCTTTCTTGGAACTAGTCTGGAGGTGCTGTTCC
AGGGCCCAGGCCATCACCACCATCACCACCATCATAGCGCCTGGTCCCACCCCCAGTTCGAG
AAGGGCGGCGGTAGTGGAGGGGGCGGATCTGGCGGCTCAGCTTGGAGCCACCCCCAGTTCGA
AAAGTGA
A5-
(SEQ ID NO: 39)
ATGTTCGTGTTTCTGGTGCTGCTGCCTCTGGTGTCCAGCACAGCTCAAGAGGGCACCTGTGG
CACCATCAGCAACAAGACCCCTCCAAACATGAACCAGTTCAGCAGCAGCAGACGGGGCGTGT
ACTACCCCGACGACATCTTCAGATCCGACGTGCTGCATCTGACCCAGGACTACTTCCTGCCT
TTCAACAGCAACGTGACCAGATACCTGAGCCTGAACGCCGACAGCAACCGGATCGTCAGATT
CGACAACCCCATCCTGCCATTCGGCGACGGCATCTATTTTGCCGCCACCGAGAAGTCCAACG
TGATCAGAGGCTGGATCTTCGGCAGCACCCTGGACAATACCAGCCAGAGCGCCATCATCGTG
AACAACAGCACCCACATCATCATCAAAGTGTGCAACTTCCAGCTGTGCGACGACCCCATGTT
CACCGTGTCTAGAGGCCAGCACTACAAGACCTGGGTGTACACAAACGCCCGGAACTGCACCT
ACGAGTACGTGTCCAAGAGCTTTCAGCTGGACGTGTCCGAGAAGAACGGCAACTTCAAGCAC
CTGAGGGAATTTGTGTTCAAGAACGTGGACGGCTTCCTGCACGTGTACAGCGCCTACGAGCC
TATCGACCTGGCTAGAGGACTGCCTAGCGGCTTCTCTGTGCTGAAGCCCATCCTGAAGCTGC
CCCTGGGCATCAACATCACCAGCTTCAGAGTCGTGATGACCATGTTCAGCCCCACCACCAGC
AATTGGCTGGCCGAAAGCGCCGCCTACTTCGTGGGATACCTGAAGCCAACCACCTTCATGCT
GAAGTTCAACGAGAACGGCACAATCACCGACGCCGTGGACTGTTCTCAGGACCCTCTGAGCG
AGCTGAAGTGCACCCTGAAGTCCTTCAACGTGGAAAAGGGCATCTACCAGACCAGCAACTTC
CGGGTGTCCCCTACACAAGAGGTCGTGCGGTTCCCCAATATCACCAATCTGTGCCCCTTCGA
CAAGGTGTTCAACGCCACCAGATTTCCCAGCGTGTACGCCTGGGAGCGCACCAAGATCTCTG
ATTGCGTGGCCGACTACACCGTGCTGTACAACTCCACCTCCTTCAGCACCTTCAAGTGCTAC
GGCGTGTCCCCAAGCAAGCTGATCGACCTGTGCTTCACCTCTGTGTACGCCGACACCTTCCT
GATCCGGTCTAGCGAAGTGCGACAGGTGGCACCTGGACAGACAGGCGTGATCGCCGATTACA
ACTACAAGCTGCCCGACGACTTCACCGGCTGTGTGATCGCCTGGAATACCGCCAAACAGGAC
GCCGGCAACTACTACTACAGAAGCCACAGAAAGACCAAGCTCAAGCCCTTCGAGCGGGACCT
GAGCAACTCCGATGAGAATGGCGTGCGGACCCTGTCCACCTACGACTTCAACCCCAATGTGC
CCATCGAGTACCAGGCCACCAGAGTGGTGGTGCTGTCCTTCGAGCTGCTGAATGCCCCTGCC
ACAGTGTGTGGCCCTAAGCTGTCTACCCAGCTGGTCAAGAACCAGTGCGTGAACTTCAACTT
CAACGGCCTGAAAGGCACCGGCGTGCTGACCGATAGCAGCAAGAGATTCCAGAGCTTCCAGC
AGTTCGGCAGGGACGCCAGCGATTTCACAGACAGCGTCAGAGATCCCCAGACACTGGAAATC
CTGGACATCAGCCCTTGCAGCTTCGGCGGAGTGTCTGTGATCACCCCTGGCACAAATGCCAG
CTCTGAAGTGGCTGTGCTGTACCAGGACGTGAACTGTACCGATGTGCCCACAGCCATCCACG
CCGATCAACTGACACCAGCTTGGCGGGTGTACTCTACCGGTGTCAACGTGTTCCAGACACAA
GCCGGCTGTCTGATTGGAGCCGAACACGTGAACGCCAGCTACGAGTGCGACATCCCTATCGG
AGCTGGAATCTGCGCCTCCTACCACACAGCCAGCACACTGAGAAGCACCGGCCAGAAATCCA
TCGTGGCCTACACAATGTCTCTGGGCGCCGAGAACTCTATCGCCTACGCCAACAACTCCATT
GCTATCCCCACCAACTTCTCCATCAGCGTGACCACCGAAGTGATGCCCGTGTCCATGGCCAA
GACCTCCGTGGATTGCACCATGTACATCTGCGGCGACAGCCAAGAGTGCAGCAACCTGCTGC
TCCAGTACGGCAGCTTCTGCACCCAGCTGAATAGAGCCCTGACCGGAATCGCCATCGAGCAG
GACAAGAACACCCAAGAGGTGTTCGCCCAAGTGAAGCAGATGTATAAGACCCCTGCCATCAA
GGACTTCGGCGGCTTCAATTTCAGCCAGATCCTGCCTGATCCTAGCAAGCCCACCAAGCGGA
GCTTCATCGAGGACCTGCTGTTCAACAAAGTGACCCTGGCCGACGCCGGCTTTATGAAGCAG
TATGGCGAGTGCCTGGGCGACATCTCTGCCAGGGATCTGATTTGCGCCCAGAAGTTTAACGG
ACTGACCGTGCTGCCTCCTCTGCTGACAGATGAGATGATCGCCGCCTATACCGCCGCACTGG
TGTCTGGTACTGCTACCGCCGGATGGACATTTGGAGCTGGTGCCGCTCTGCAGATCCCCTTC
GCTATGCAGATGGCCTACAGATTCAACGGCATCGGCGTGACCCAGAACGTGCTGTATGAGAA
CCAGAAGCAGATCGCCAATCAGTTCAACAAGGCCATCAGTCAGATCCAAGAGAGCCTGACCA
CCACAAGCACAGCCCTGGGAAAGCTGCAGGACGTGGTCAACCAGAATGCCCAGGCTCTGAAC
ACCCTGGTCAAGCAGCTGAGCAGCAATTTCGGCGCCATCAGCAGCGTGCTGAACGACATCCT
GAGCCGGCTGGATAAGGTGGAAGCCGAGGTGCAGATCGACCGGCTGATTACAGGCAGACTGC
AGAGCCTGCAGACCTACGTGACACAGCAGCTGATCAGAGCCGCCGAGATTAGAGCCTCTGCC
AATCTGGCCGCCACCAAGATGAGCGAGTGTGTGCTGGGCCAGAGCAAGAGAGTGGACTTTTG
CGGCAAGGGCTACCACCTGATGAGCTTCCCTCAAGCCGCTCCTCACGGCGTGGTGTTTCTGC
ACGTGACATACGTGCCAAGCCAAGAGCGGAACTTCACCACCGCTCCAGCCATTTGCCACGAG
GGCAAAGCCTACTTTCCCCGCGAAGGCGTGTTCGTGTCTAACGGCACCTCCTGGTTCATCAC
CCAGAGGAACTTCTACAGCCCTCAGATCATCACCACCGACAACACCTTCGTGGCCGGCAATT
GCGACGTCGTGATCGGCATCATCAACAATACCGTGTACGACCCTCTGCAGCCCGAGCTGGAC
AGCTTCAAAGAGGAACTGGACAAGTACTTCAAGAATCACACAAGCCCCGACGTGGACCTGGG
CGATATCAGCGGAATCAATGCCAGCGTCGTGAACATCCAGAAAGAGATCGACAGACTGAACG
AGGTGGCCAAGAACCTGAACGAGTCCCTGATTGACCTGCAAGAGCTGGGGAAGTACGAGCAA
GGATCTGGCTATATTCCCGAGGCTCCTAGAGATGGCCAGGCCTACGTTAGAAAGGACGGCGA
GTGGGTCCTGCTGAGCACCTTTCTTGGAACTAGTCTGGAGGTGCTGTTCCAGGGCCCAGGCC
ATCACCACCATCACCACCATCATAGCGCCTGGTCCCACCCCCAGTTCGAGAAGGGCGGCGGT
AGTGGAGGGGGCGGATCTGGCGGCTCAGCTTGGAGCCACCCCCAGTTCGAAAAGTGA
A6-
(SEQ ID NO: 40)
ATGTTCGTGTTTCTGGTGCTGCTGCCCCTGGTGTCCTCTACAGCTCAAGAGGGCACATGTGG
CACCCTGAGCAACAAGAGCCCTCCTAACATGACCCAGTTCAGCAGCTCTCGGAGAGGCGTGT
ACTACCCCGACGACATCTTCAGATCCGACGTGCTGCATCTGACCCAGGACTACTTCCTGCCT
TTCAACAGCAACGTGACCAGATACCTGAGCCTGAACAGCGACAGCGACCGGATCGTCAGATT
CGACAACCCTATCATCCCCTTCGGCGACGGGGTGTACTTTGCCGCCACCGAGAAGTCCAACG
TGATCAGAGGCTGGATCTTCGGCAGCACCCTGGACAATACCAGCCAGAGCGCCATCATCATG
AACAACAGCACCCACATCGTGATTCGCGTGTGCAACTTCCAGCTGTGCGACGACCCTATGTT
CGCCGTGTCTAGACCTACCGGCCAGCACTACAAGACCTGGATCTACACCAACGCCAGAAACT
GCACCTACGAGTACGTGTCCAAGAGCTTTCAGCTGGACGTGTCCGAGAAGCCCGGCAACTTC
AAACACCTGAGGGAATTTGTGTTCAAGAACGTGGACGGCTTCCTGCACGTGTACAGCGGCTA
CGAGCCTATCGATGTGGCCAGAGGACTGCCTAGCGGCTTCTCTGTGCTGAAGCCCATCTTCA
AGCTGCCTCTGGGCATCAACATCACCAACTTCAGAGTGATCATGACCATGTTCAGCCCCACC
ACCAGCAACTGGGGAGCTGAAGCCGCCGCTTACTTCGTGGGCTACCTGAAGCCTACCACCTT
CATGCTGAAGTTCGACGAGAACGGCACCATCACCGACGCCGTGGACTGTAGCCAAGATCCTC
TGAGCGAGCTGAAGTGCACCGTGAAGTCCTTCAACGTGGAAAAGGGCATCTACCAGACCAGC
AATTTCCGGGTGTCCCCTACCAAAGAAGTCGTGCGGTTCCCCAATATCACCAATCTGTGCCC
TTTCGGCGAGGTGTTCAACGCCACCACCTTTCCATCTGTGTACGCCTGGGAGAGAACCCGGA
TCAGCGATTGCGTGGCCGATTACAGCGTGCTGTACAACTCCACCAGCTTCTCCACCTTCAAG
TGCTACGGCGTGTCACCCACCAAGCTGAACGACCTGTGCTTCAGCAGCGTGTACGCCGACAG
CTTTGTGGTCAAGGGCGACGATGTGCGGCAGATTGCTCCTGGACAGACAGGCGTGATCGCCG
ACTACAACTACAAGCTGCCCGACGACTTCACCGGCTGTGTGATCGCCTGGAACACCGCCAAT
CTGGATGCCACCAGCACCGGCAACTACAATTACTACTACAGAAGCCTGCGGCACGGCAAGCT
GAAACCCTTCGAGAGGGACATCTCCAACGTGCCATTCAGCCCTGAGGGCAAGCCTTGTACAC
CTCCAGCCTTCAACTGTTACAGACCCCTGAACACCTACGGCTTCAACCCCACAGTCGGCATC
GGCTACCAGCCTTACAGAGTGGTGGTGCTGAGCTTCGAACTGCTGAATGCCCCTGCCACAGT
GTGCGGCCCTAAGCTGTCTACAGAGCTGGTCAAGAACCAGTGCGTGAACTTCAACTTCAACG
GCCTGACCGGCACCGGCGTGCTGACAGATAGCAGCAAGAGATTCCAGCCTTTCCAGCAGTTT
GGCCGGGATGTGTCCGATTTCACCGATAGCGTGCGGGACCCCAAGACACTGGAAATCCTGGA
CATCAGCCCCTGCAGCTTTGGCGGAGTGTCTGTGATCACCCCTGGCACCAATACCTCTAGCG
AAGTGGCCGTGCTGTATCAGGACGTGAACTGCACCGATGTGCCCACAGCCATTCACGCCGAT
CAGCTGACACCAGCTTGGAGAGTGTACTCTACCGGTGTCAACGTGTTCCAGACACAAGCCGG
CTGTCTGATTGGAGCCGAACACGTGAACGCCAGCTACGAGTGCGACATCCCTATCGGAGCTG
GCATCTGTGCCAGCTACCACACAGCCAGCACACTGAGAAGCACCGGCCAGAAATCCATCGTG
GCCTACACAATGTCTCTGGGCGCCGAGAACTCTATCGCCTACAGCAACAACACAATCGCTAT
CCCCACCAATTTCAGCATCAGCGTGACCACCGAAGTGATGCCCGTGTCCATGGCCAAGACCT
CCGTGGATTGCACCATGTACATCTGCGGCGACAGCACCGAGTGCAGCAACCTGCTGCTGCAG
TACGGCAGCTTCTGCACCCAGCTGAATAGAGCCCTGAGCGGAATTGCCGTGGAACAGGACAA
GAACACCCGGGAAGTGTTCGCCCAAGTGAAGCAGATGTATAAGACCCCTGCCATCAAGGACT
TCGGCGGCTTTAACTTCAGCCAGATCCTGCCTGATCCTAGCAAGCCCACCAAGAGAAGCTTC
ATCGAGGACCTGCTGTTCAACAAAGTGACCCTGGCCGACGCCGGCTTTATGAAGCAGTATGG
CGAATGCCTGGGCGACATCAGCGCCAGGGATCTGATTTGCGCCCAGAAGTTTAACGGACTGA
CCGTGCTGCCTCCTCTGCTGACCGATGAGATGATCGCCGCCTATACAGCCGCTCTGGTGTCT
GGCACAGCTACCGCCGGATGGACATTTGGAGCTGGCGCTGCTCTGCAGATCCCCTTTGCTAT
GCAGATGGCCTACAGATTCAATGGCATCGGAGTGACCCAGAATGTGCTGTACGAGAACCAGA
AGCAGATCGCCAACCAGTTCAACAAGGCCATCAGTCAGATCCAAGAGAGCCTGACCACCACA
AGCACAGCCCTGGGCAAACTGCAGGACGTGGTCAACCAGAATGCCCAGGCTCTGAACACCCT
GGTCAAGCAGCTGAGCAGCAACTTCGGCGCCATCAGCTCCGTGCTGAACGATATCCTGAGCC
GGCTGGACAAGGTGGAAGCCGAGGTGCAGATCGACAGACTGATCACCGGAAGGCTGCAGAGC
CTGCAGACCTACGTTACCCAGCAGCTGATCAGAGCCGCCGAGATTAGAGCCAGCGCTAATCT
GGCCGCCACCAAGATGTCTGAGTGTGTGCTGGGCCAGAGCAAGAGAGTGGACTTTTGCGGCA
AGGGCTACCACCTGATGAGCTTCCCTCAAGCCGCTCCTCACGGCGTGGTGTTTCTGCACGTG
ACATACGTGCCAAGCCAAGAGCGGAACTTCACCACCGCTCCAGCCATTTGCCACGAGGGCAA
AGCCTACTTTCCCCGCGAAGGCGTGTTCGTGTCTAACGGCACAAGCTGGTTCATCACCCAGC
GCAACTTCTACAGCCCTCAGATCATCACCACCGACAACACCTTCGTGTCCGGCAACTGCGAC
GTCGTGATCGGCATCATCAACAATACCGTGTACGACCCACTGCAGCCCGAGCTGGACAGCTT
CAAAGAGGAACTGGACAAGTACTTCAAGAATCACACAAGCCCCGACGTGGACCTGGGCGATA
TCTCTGGCATCAATGCCTCCGTCGTGAACATCCAGAAAGAGATCGACCGGCTGAACGAGGTG
GCCAAGAACCTGAACGAGTCCCTGATCGACCTGCAAGAGCTGGGGAAGTACGAGCAAGGATC
TGGCTATATTCCCGAGGCTCCTAGAGATGGCCAGGCCTACGTTAGAAAGGACGGCGAGTGGG
TCCTGCTGAGCACCTTTCTTGGAACTAGTCTGGAGGTGCTGTTCCAGGGCCCAGGCCATCAC
CACCATCACCACCATCATAGCGCCTGGTCCCACCCCCAGTTCGAGAAGGGCGGCGGTAGTGG
AGGGGGCGGATCTGGCGGCTCAGCTTGGAGCCACCCCCAGTTCGAAAAGTGA
A3 with wt RBD-
(SEQ ID NO: 41)
ATGTTCGTGTTTCTGGTGCTGCTGCCTCTGGTGTCCAGCCAGTGTGTGAATCTGACCGGCAG
AACCCCTCTGAACCCCAACTACACCAACAGCAGCCAGCGGGGCGTGTACTACCCCGACACCA
TCTTTAGAAGCGACACCCTGGTGCTGAGCCAGGGCTACTTCCTGCCTTTCTACAGCAACGTG
TCCTGGTACTACAGCCTGACCACCAACAACGCCGCCACCAAGAGATTCGACAACCCCATCCT
GGACTTCAAGGACGGCATCTACTTTGCCGCCACCGAGAAGTCCAACATCATCAGAGGCTGGA
TCTTCGGCACCACACTGGACAACACAAGCCAGAGCCTGCTGATCGTGAACAACGCCACCAAC
GTGATCATCAAAGTGTGCAACTTCCAGTTCTGCTACGACCCCTACCTGAGCGGCTACTACGG
CCACAACAACAAGACCTGGTCCATCCGCGAGTTCGCCGTGTACAGCAGCTACGCCAATTGCA
CCTTCGAGTACGTGTCCAAGAGCTTCATGCTGGACATCAGCGGCAAAGGCGGCCTGTTCAAT
ACCCTGCGCGAGTTTGTGTTCAGAAACGTGGACGGCTACTTCAAGATCTACTCCAAGTACAC
CCCTGTGAACCTGAACCGGGGCCTGCCTACAGGCTTTTCTGTTCTGCAGCCCCTGGTGGAAC
TGCCCGTGGGCATCAACATCACCAAGTTCAGAACCCTGCTGACCATCCACAGAGGCGACCCC
ATGCCTAACAATGGCTGGACCGCCTTTAGCGCCGCCTACTTCGTGGGCTACCTGAAGCCTCG
GACCTTTATGCTGAAGTACAACGAGAACGGCACCATCACCGACGCCGTGGATTGTGCCCTTG
ATCCTCTGAGCGAGACAAAGTGCACCCTGAAGTCCCTGACAGTGCAGAAGGGCATCTACCAG
ACCAGCAACTTCCGGGTGCAGCCCACCGAATCCATCGTGCGGTTCCCCAATATCACCAATCT
GTGCCCCTTCGGCGAGGTGTTCAATGCCACCAGATTCGCCTCTGTGTACGCCTGGAACCGGA
AGCGGATCAGCAATTGCGTGGCCGACTACTCCGTGCTGTACAACTCCGCCAGCTTCAGCACC
TTCAAGTGCTACGGCGTGTCCCCTACCAAGCTGAACGACCTGTGCTTCACAAACGTGTACGC
CGACAGCTTCGTGATCCGGGGAGATGAAGTGCGGCAGATTGCCCCTGGACAGACAGGCAAGA
TCGCCGACTACAACTACAAGCTGCCCGACGACTTCACCGGCTGTGTGATTGCCTGGAACAGC
AACAACCTGGACTCCAAAGTCGGCGGCAACTACAATTACCTGTACCGGCTGTTCCGGAAGTC
CAATCTGAAGCCCTTCGAGCGGGACATCTCCACCGAGATCTATCAGGCCGGCAGCACCCCTT
GTAACGGCGTGGAAGGCTTCAACTGCTACTTCCCACTGCAGTCCTACGGCTTTCAGCCCACA
AATGGCGTGGGCTATCAGCCCTACAGAGTGGTGGTGCTGAGCTTCGAACTGCTGCATGCCCC
TGCCACAGTGTGCGGCCCTAAGAAAAGCACCAATCTCGTGAAGAACAAATGCGTGAACTTCA
ACTTCAACGGCCTGAAAGGCACCGGCGTGCTGACCGATAGCAGCAAGAGATTTCAGAGCTTC
CAGCAGTTCGGCCGGGACGCCAGCGATTTCACAGACAGCGTTAGAGATCCCCAGACACTGGA
AATCCTGGATATCACCCCTTGCAGCTTCGGCGGAGTGTCCGTGATCACCCCTGGCACAAATA
CCAGCTCTGAGGTGGCCGTGCTGTACCAGGATGTGAACTGCACCGATGTGCCCACAGCCATC
CATGCCGATCAGCTGACACCCGCCTGGCGGATCTATAGCACCGGCACCAATGTGTTTCAGAC
CCAAGCCGGCTGTCTGATTGGAGCCGAGCATGTGAATGCCAGCTACGAGTGCGACATCCCTA
TCGGAGCCGGCATCTGCGCCAGCTACCACACAGCCTCTATCCTGAGATCCACCAGCCAGAAA
GCCATCGTGGCCTACACAATGTCTCTGGGCGCCGAGAACTCTATCGCCTACGCCAACAACAG
CATTGCTATCCCCACCAACTTCTCCATCAGCGTGACCACCGAAGTGATGCCCGTGTCCATGG
CCAAGACCAGCGTGGACTGCACCATGTACATCTGCGGCGACAGCATCGAGTGCAGCAACCTG
CTGCTGCAGTACGGCAGCTTCTGCACCCAGCTGAATAGAGCCCTGACCGGAATCGCCATCGA
GCAGGACAAGAACACCCAAGAGGTGTTCGCCCAAGTGAAGCAGATCTACAAGACCCCTCCTA
TCAAGGACTTCGGCGGCTTCAATTTCAGCCAGATCCTGCCAGATCCAAGCAAGCCCAGCAAG
CGGAGCTTCATCGAGGACCTGCTGTTCAACAAAGTGACACTGGCCGACGCCGGCTTCATCAA
GCAGTATGGCGATTGCCTGGGCGACATCTCCGCCAGGGATCTGATTTGCGCCCAGAAGTTCA
ACGGACTGACCGTGCTGCCTCCTCTGCTGACAGATGAGATGATCGCCGCCTATACCGCCGCT
CTGATCTCTGGAACAGCCACCGCCGGATGGACATTTGGAGCTGGTGCAGCTCTGCAGATCCC
CTTCGCTATGCAGATGGCCTACCGGTTCAATGGCATCGGCGTGACCCAGAATGTGCTGTACG
AGAACCAGAAGCTGATTGCCAACCAGTTCAACAGCGCCATCGGCAAGATCCAAGAGAGCCTG
ACCTCTACCGCCAGCGCTCTGGGAAAGCTGCAGGACGTCGTGAACCAGAACGCCCAGGCTCT
GAACACCCTCGTGAAGCAGCTGAGCAGCAATTTCGGCGCCATCAGCAGCGTGCTGAACGACA
TCCTGAGCCGGCTGGATAAGGTGGAAGCCGAGGTGCAGATCGACCGGCTGATTACAGGCAGA
CTGCAGAGCCTCCAGACCTACGTGACACAGCAGCTGATCAGAGCCGCCGAGATTAGAGCCTC
TGCCAATCTGGCCGCCACAAAGATGAGCGAGTGTGTGCTGGGCCAGAGCAAGAGAGTGGACT
TTTGCGGCAAGGGCTACCACCTGATGAGCTTCCCTCAGTCTGCTCCTCACGGCGTGGTGTTT
CTGCACGTGACATACGTGCCCAGCCAAGAGAAGAACTTCACCACCGCTCCAGCCATCTGCCA
CGAGGGCAAAGCCCACTTTCCTAGAGAAGGCGTGTTCGTGTCCAACGGCACCCATTGGTTCG
TGACTCAGCGGAACTTCTACGAGCCCCAGATCATCACCACCGACAACACCTTCGTGTCCGGC
AACTGCGACGTGGTCATCGGCATCATCAACAATACCGTGTACGACCCACTGCAGCCCGAGCT
GGACAGCTTCAAAGAGGAACTGGACAAGTACTTTAAGAACCACACAAGCCCCGACGTGGACC
TGGGCGATATCAGCGGAATCAATGCCAGCGTGGTCAACATCCAGAAAGAGATCGATAGGCTG
AACGAGGTGGCCAAGAACCTGAATGAGTCCCTGATTGACCTGCAAGAGCTGGGGAAGTACGA
GCAAGGATCTGGCTATATTCCCGAGGCTCCTAGAGATGGCCAGGCCTACGTTAGAAAGGACG
GCGAGTGGGTCCTGCTGAGCACCTTTCTTGGAACTAGTCTGGAGGTGCTGTTCCAGGGCCCA
GGCCATCACCACCATCACCACCATCATAGCGCCTGGTCCCACCCCCAGTTCGAGAAGGGCGG
CGGTAGTGGAGGGGGCGGATCTGGCGGCTCAGCTTGGAGCCACCCCCAGTTCGAAAAGTGA
A5 with wt RBD-
(SEQ ID NO: 42)
ATGTTCGTGTTTCTGGTGCTGCTGCCTCTGGTGTCCAGCACAGCTCAAGAGGGCACCTGTGG
CACCATCAGCAACAAGACCCCTCCAAACATGAACCAGTTCAGCAGCAGCAGACGGGGCGTGT
ACTACCCCGACGACATCTTCAGATCCGACGTGCTGCATCTGACCCAGGACTACTTCCTGCCT
TTCAACAGCAACGTGACCAGATACCTGAGCCTGAACGCCGACAGCAACCGGATCGTCAGATT
CGACAACCCCATCCTGCCATTCGGCGACGGCATCTATTTTGCCGCCACCGAGAAGTCCAACG
TGATCAGAGGCTGGATCTTCGGCAGCACCCTGGACAATACCAGCCAGAGCGCCATCATCGTG
AACAACAGCACCCACATCATCATCAAAGTGTGCAACTTCCAGCTGTGCGACGACCCCATGTT
CACCGTGTCTAGAGGCCAGCACTACAAGACCTGGGTGTACACAAACGCCCGGAACTGCACCT
ACGAGTACGTGTCCAAGAGCTTTCAGCTGGACGTGTCCGAGAAGAACGGCAACTTCAAGCAC
CTGAGGGAATTTGTGTTCAAGAACGTGGACGGCTTCCTGCACGTGTACAGCGCCTACGAGCC
TATCGACCTGGCTAGAGGACTGCCTAGCGGCTTCTCTGTGCTGAAGCCCATCCTGAAGCTGC
CCCTGGGCATCAACATCACCAGCTTCAGAGTCGTGATGACCATGTTCAGCCCCACCACCAGC
AATTGGCTGGCCGAAAGCGCCGCCTACTTCGTGGGATACCTGAAGCCAACCACCTTCATGCT
GAAGTTCAACGAGAACGGCACAATCACCGACGCCGTGGACTGTTCTCAGGACCCTCTGAGCG
AGCTGAAGTGCACCCTGAAGTCCTTCAACGTGGAAAAGGGCATCTACCAGACCAGCAACTTC
CGGGTGCAGCCCACCGAATCCATCGTGCGGTTCCCCAATATCACCAATCTGTGCCCCTTCGG
CGAGGTGTTCAATGCCACCAGATTCGCCTCTGTGTACGCCTGGAACCGGAAGCGGATCAGCA
ATTGCGTGGCCGACTACTCCGTGCTGTACAACTCCGCCAGCTTCAGCACCTTCAAGTGCTAC
GGCGTGTCCCCTACCAAGCTGAACGACCTGTGCTTCACAAACGTGTACGCCGACAGCTTCGT
GATCCGGGGAGATGAAGTGCGGCAGATTGCCCCTGGACAGACAGGCAAGATCGCCGACTACA
ACTACAAGCTGCCCGACGACTTCACCGGCTGTGTGATTGCCTGGAACAGCAACAACCTGGAC
TCCAAAGTCGGCGGCAACTACAATTACCTGTACCGGCTGTTCCGGAAGTCCAATCTGAAGCC
CTTCGAGCGGGACATCTCCACCGAGATCTATCAGGCCGGCAGCACCCCTTGTAACGGCGTGG
AAGGCTTCAACTGCTACTTCCCACTGCAGTCCTACGGCTTTCAGCCCACAAATGGCGTGGGC
TATCAGCCCTACAGAGTGGTGGTGCTGAGCTTCGAACTGCTGCATGCCCCTGCCACAGTGTG
CGGCCCTAAGAAAAGCACCAATCTCGTGAAGAACAAATGCGTGAACTTCAACTTCAACGGCC
TGAAAGGCACCGGCGTGCTGACCGATAGCAGCAAGAGATTCCAGAGCTTCCAGCAGTTCGGC
AGGGACGCCAGCGATTTCACAGACAGCGTCAGAGATCCCCAGACACTGGAAATCCTGGACAT
CAGCCCTTGCAGCTTCGGCGGAGTGTCTGTGATCACCCCTGGCACAAATGCCAGCTCTGAAG
TGGCTGTGCTGTACCAGGACGTGAACTGTACCGATGTGCCCACAGCCATCCACGCCGATCAA
CTGACACCAGCTTGGCGGGTGTACTCTACCGGTGTCAACGTGTTCCAGACACAAGCCGGCTG
TCTGATTGGAGCCGAACACGTGAACGCCAGCTACGAGTGCGACATCCCTATCGGAGCTGGAA
TCTGCGCCTCCTACCACACAGCCAGCACACTGAGAAGCACCGGCCAGAAATCCATCGTGGCC
TACACAATGTCTCTGGGCGCCGAGAACTCTATCGCCTACGCCAACAACTCCATTGCTATCCC
CACCAACTTCTCCATCAGCGTGACCACCGAAGTGATGCCCGTGTCCATGGCCAAGACCTCCG
TGGATTGCACCATGTACATCTGCGGCGACAGCCAAGAGTGCAGCAACCTGCTGCTCCAGTAC
GGCAGCTTCTGCACCCAGCTGAATAGAGCCCTGACCGGAATCGCCATCGAGCAGGACAAGAA
CACCCAAGAGGTGTTCGCCCAAGTGAAGCAGATGTATAAGACCCCTGCCATCAAGGACTTCG
GCGGCTTCAATTTCAGCCAGATCCTGCCTGATCCTAGCAAGCCCACCAAGCGGAGCTTCATC
GAGGACCTGCTGTTCAACAAAGTGACCCTGGCCGACGCCGGCTTTATGAAGCAGTATGGCGA
GTGCCTGGGCGACATCTCTGCCAGGGATCTGATTTGCGCCCAGAAGTTTAACGGACTGACCG
TGCTGCCTCCTCTGCTGACAGATGAGATGATCGCCGCCTATACCGCCGCACTGGTGTCTGGT
ACTGCTACCGCCGGATGGACATTTGGAGCTGGTGCCGCTCTGCAGATCCCCTTCGCTATGCA
GATGGCCTACAGATTCAACGGCATCGGCGTGACCCAGAACGTGCTGTATGAGAACCAGAAGC
AGATCGCCAATCAGTTCAACAAGGCCATCAGTCAGATCCAAGAGAGCCTGACCACCACAAGC
ACAGCCCTGGGAAAGCTGCAGGACGTGGTCAACCAGAATGCCCAGGCTCTGAACACCCTGGT
CAAGCAGCTGAGCAGCAATTTCGGCGCCATCAGCAGCGTGCTGAACGACATCCTGAGCCGGC
TGGATAAGGTGGAAGCCGAGGTGCAGATCGACCGGCTGATTACAGGCAGACTGCAGAGCCTG
CAGACCTACGTGACACAGCAGCTGATCAGAGCCGCCGAGATTAGAGCCTCTGCCAATCTGGC
CGCCACCAAGATGAGCGAGTGTGTGCTGGGCCAGAGCAAGAGAGTGGACTTTTGCGGCAAGG
GCTACCACCTGATGAGCTTCCCTCAAGCCGCTCCTCACGGCGTGGTGTTTCTGCACGTGACA
TACGTGCCAAGCCAAGAGCGGAACTTCACCACCGCTCCAGCCATTTGCCACGAGGGCAAAGC
CTACTTTCCCCGCGAAGGCGTGTTCGTGTCTAACGGCACCTCCTGGTTCATCACCCAGAGGA
ACTTCTACAGCCCTCAGATCATCACCACCGACAACACCTTCGTGGCCGGCAATTGCGACGTC
GTGATCGGCATCATCAACAATACCGTGTACGACCCTCTGCAGCCCGAGCTGGACAGCTTCAA
AGAGGAACTGGACAAGTACTTCAAGAATCACACAAGCCCCGACGTGGACCTGGGCGATATCA
GCGGAATCAATGCCAGCGTCGTGAACATCCAGAAAGAGATCGACAGACTGAACGAGGTGGCC
AAGAACCTGAACGAGTCCCTGATTGACCTGCAAGAGCTGGGGAAGTACGAGCAAGGATCTGG
CTATATTCCCGAGGCTCCTAGAGATGGCCAGGCCTACGTTAGAAAGGACGGCGAGTGGGTCC
TGCTGAGCACCTTTCTTGGAACTAGTCTGGAGGTGCTGTTCCAGGGCCCAGGCCATCACCAC
CATCACCACCATCATAGCGCCTGGTCCCACCCCCAGTTCGAGAAGGGCGGCGGTAGTGGAGG
GGGCGGATCTGGCGGCTCAGCTTGGAGCCACCCCCAGTTCGAAAAGTGA
A6 with wt RBD-
(SEQ ID NO: 43)
ATGTTCGTGTTTCTGGTGCTGCTGCCCCTGGTGTCCTCTACAGCTCAAGAGGGCACATGTGG
CACCCTGAGCAACAAGAGCCCTCCTAACATGACCCAGTTCAGCAGCTCTCGGAGAGGCGTGT
ACTACCCCGACGACATCTTCAGATCCGACGTGCTGCATCTGACCCAGGACTACTTCCTGCCT
TTCAACAGCAACGTGACCAGATACCTGAGCCTGAACAGCGACAGCGACCGGATCGTCAGATT
CGACAACCCTATCATCCCCTTCGGCGACGGGGTGTACTTTGCCGCCACCGAGAAGTCCAACG
TGATCAGAGGCTGGATCTTCGGCAGCACCCTGGACAATACCAGCCAGAGCGCCATCATCATG
AACAACAGCACCCACATCGTGATTCGCGTGTGCAACTTCCAGCTGTGCGACGACCCTATGTT
CGCCGTGTCTAGACCTACCGGCCAGCACTACAAGACCTGGATCTACACCAACGCCAGAAACT
GCACCTACGAGTACGTGTCCAAGAGCTTTCAGCTGGACGTGTCCGAGAAGCCCGGCAACTTC
AAACACCTGAGGGAATTTGTGTTCAAGAACGTGGACGGCTTCCTGCACGTGTACAGCGGCTA
CGAGCCTATCGATGTGGCCAGAGGACTGCCTAGCGGCTTCTCTGTGCTGAAGCCCATCTTCA
AGCTGCCTCTGGGCATCAACATCACCAACTTCAGAGTGATCATGACCATGTTCAGCCCCACC
ACCAGCAACTGGGGAGCTGAAGCCGCCGCTTACTTCGTGGGCTACCTGAAGCCTACCACCTT
CATGCTGAAGTTCGACGAGAACGGCACCATCACCGACGCCGTGGACTGTAGCCAAGATCCTC
TGAGCGAGCTGAAGTGCACCGTGAAGTCCTTCAACGTGGAAAAGGGCATCTACCAGACCAGC
AATTTCCGGGTGCAGCCCACCGAATCCATCGTGCGGTTCCCCAATATCACCAATCTGTGCCC
CTTCGGCGAGGTGTTCAATGCCACCAGATTCGCCTCTGTGTACGCCTGGAACCGGAAGCGGA
TCAGCAATTGCGTGGCCGACTACTCCGTGCTGTACAACTCCGCCAGCTTCAGCACCTTCAAG
TGCTACGGCGTGTCCCCTACCAAGCTGAACGACCTGTGCTTCACAAACGTGTACGCCGACAG
CTTCGTGATCCGGGGAGATGAAGTGCGGCAGATTGCCCCTGGACAGACAGGCAAGATCGCCG
ACTACAACTACAAGCTGCCCGACGACTTCACCGGCTGTGTGATTGCCTGGAACAGCAACAAC
CTGGACTCCAAAGTCGGCGGCAACTACAATTACCTGTACCGGCTGTTCCGGAAGTCCAATCT
GAAGCCCTTCGAGCGGGACATCTCCACCGAGATCTATCAGGCCGGCAGCACCCCTTGTAACG
GCGTGGAAGGCTTCAACTGCTACTTCCCACTGCAGTCCTACGGCTTTCAGCCCACAAATGGC
GTGGGCTATCAGCCCTACAGAGTGGTGGTGCTGAGCTTCGAACTGCTGCATGCCCCTGCCAC
AGTGTGCGGCCCTAAGAAAAGCACCAATCTCGTGAAGAACAAATGCGTGAACTTCAACTTCA
ACGGCCTGACCGGCACCGGCGTGCTGACAGATAGCAGCAAGAGATTCCAGCCTTTCCAGCAG
TTTGGCCGGGATGTGTCCGATTTCACCGATAGCGTGCGGGACCCCAAGACACTGGAAATCCT
GGACATCAGCCCCTGCAGCTTTGGCGGAGTGTCTGTGATCACCCCTGGCACCAATACCTCTA
GCGAAGTGGCCGTGCTGTATCAGGACGTGAACTGCACCGATGTGCCCACAGCCATTCACGCC
GATCAGCTGACACCAGCTTGGAGAGTGTACTCTACCGGTGTCAACGTGTTCCAGACACAAGC
CGGCTGTCTGATTGGAGCCGAACACGTGAACGCCAGCTACGAGTGCGACATCCCTATCGGAG
CTGGCATCTGTGCCAGCTACCACACAGCCAGCACACTGAGAAGCACCGGCCAGAAATCCATC
GTGGCCTACACAATGTCTCTGGGCGCCGAGAACTCTATCGCCTACAGCAACAACACAATCGC
TATCCCCACCAATTTCAGCATCAGCGTGACCACCGAAGTGATGCCCGTGTCCATGGCCAAGA
CCTCCGTGGATTGCACCATGTACATCTGCGGCGACAGCACCGAGTGCAGCAACCTGCTGCTG
CAGTACGGCAGCTTCTGCACCCAGCTGAATAGAGCCCTGAGCGGAATTGCCGTGGAACAGGA
CAAGAACACCCGGGAAGTGTTCGCCCAAGTGAAGCAGATGTATAAGACCCCTGCCATCAAGG
ACTTCGGCGGCTTTAACTTCAGCCAGATCCTGCCTGATCCTAGCAAGCCCACCAAGAGAAGC
TTCATCGAGGACCTGCTGTTCAACAAAGTGACCCTGGCCGACGCCGGCTTTATGAAGCAGTA
TGGCGAATGCCTGGGCGACATCAGCGCCAGGGATCTGATTTGCGCCCAGAAGTTTAACGGAC
TGACCGTGCTGCCTCCTCTGCTGACCGATGAGATGATCGCCGCCTATACAGCCGCTCTGGTG
TCTGGCACAGCTACCGCCGGATGGACATTTGGAGCTGGCGCTGCTCTGCAGATCCCCTTTGC
TATGCAGATGGCCTACAGATTCAATGGCATCGGAGTGACCCAGAATGTGCTGTACGAGAACC
AGAAGCAGATCGCCAACCAGTTCAACAAGGCCATCAGTCAGATCCAAGAGAGCCTGACCACC
ACAAGCACAGCCCTGGGCAAACTGCAGGACGTGGTCAACCAGAATGCCCAGGCTCTGAACAC
CCTGGTCAAGCAGCTGAGCAGCAACTTCGGCGCCATCAGCTCCGTGCTGAACGATATCCTGA
GCCGGCTGGACAAGGTGGAAGCCGAGGTGCAGATCGACAGACTGATCACCGGAAGGCTGCAG
AGCCTGCAGACCTACGTTACCCAGCAGCTGATCAGAGCCGCCGAGATTAGAGCCAGCGCTAA
TCTGGCCGCCACCAAGATGTCTGAGTGTGTGCTGGGCCAGAGCAAGAGAGTGGACTTTTGCG
GCAAGGGCTACCACCTGATGAGCTTCCCTCAAGCCGCTCCTCACGGCGTGGTGTTTCTGCAC
GTGACATACGTGCCAAGCCAAGAGCGGAACTTCACCACCGCTCCAGCCATTTGCCACGAGGG
CAAAGCCTACTTTCCCCGCGAAGGCGTGTTCGTGTCTAACGGCACAAGCTGGTTCATCACCC
AGCGCAACTTCTACAGCCCTCAGATCATCACCACCGACAACACCTTCGTGTCCGGCAACTGC
GACGTCGTGATCGGCATCATCAACAATACCGTGTACGACCCACTGCAGCCCGAGCTGGACAG
CTTCAAAGAGGAACTGGACAAGTACTTCAAGAATCACACAAGCCCCGACGTGGACCTGGGCG
ATATCTCTGGCATCAATGCCTCCGTCGTGAACATCCAGAAAGAGATCGACCGGCTGAACGAG
GTGGCCAAGAACCTGAACGAGTCCCTGATCGACCTGCAAGAGCTGGGGAAGTACGAGCAAGG
ATCTGGCTATATTCCCGAGGCTCCTAGAGATGGCCAGGCCTACGTTAGAAAGGACGGCGAGT
GGGTCCTGCTGAGCACCTTTCTTGGAACTAGTCTGGAGGTGCTGTTCCAGGGCCCAGGCCAT
CACCACCATCACCACCATCATAGCGCCTGGTCCCACCCCCAGTTCGAGAAGGGCGGCGGTAG
TGGAGGGGGCGGATCTGGCGGCTCAGCTTGGAGCCACCCCCAGTTCGAAAAGTGA

Hence, in an embodiment, the nucleic acid molecule is selected from the group consisting of SEQ ID NO: 38 to 43, in particular from the group consisting of SEQ ID NO: 41 to 43 and variants thereof. A variant of any of SEQ ID NO: 38 to 43, or indeed SEQ ID NO: 41 to 43 as used herein includes a nucleic acid molecule encoding for the same coronavirus spike protein, as the nucleic acid molecule as defined in any of SEQ ID NO: 38 to 43, or indeed SEQ ID NO: 41 to 43 but may have at least one synonymous substitution, i.e., substitution of at least one base or nucleotide for another base or nucleotide such that the produced amino acid sequence is not modified. Hence, such a synonymous substitution changes at least one base in a codon in the nucleic acid molecule into another codon, which both encode for the same amino acid residue. For instance, a nucleic acid molecule according to any of SEQ ID NO: 38 to 43, or indeed SEQ ID NO: 41 to 43 could be codon optimized for expression in a particular host cell.

In an embodiment, the nucleic acid molecule is an isolated nucleic acid molecule.

The present invention also relates to an expression vector comprising a nucleic acid molecule according to the invention. The expression vector preferably also comprises a promoter. In such a case, the nucleic acid molecule is operably connected to and under transcriptional control of the promoter. The expression vector may optionally comprise other regulatory elements, such as an enhancer.

The expression vector may be a self-replicating nucleic acid structure or an expression vector to be incorporated into the genome of a host cell into which it has been introduced. Examples of expression vectors include a plasmid, an episomal plasmid and a virus vector. Non-limiting, but illustrative, examples of virus vectors include a lentiviral vector, an adenoviral vector, an adeno-associated viral vector, a retroviral vector, a Semliki Forest virus vector, a polio virus and a hybrid vector.

In an embodiment, the expression vector is an isolated expression vector.

The expression vector may be introduced into a host cell for protein expression and/or propagation of the vector comprising the nucleic acid molecule. Also provided herein is, thus, a host cell comprising the expression vector. The host cell used can be any type of host cell, including both eukaryotic and prokaryotic host cells. Examples of the former include yeast cells, mammalian cells and human cells, such as human cell line cells, whereas bacterial cells are examples of prokaryotic host cells. In a particular embodiment, the host cell is a microbial cell. Host cell as used herein includes “transformants”, “transformed cells” and “transfected cells”, including “transiently transfected cells” and “stably transfected cells”, which include the primary transformed or transfected cell and progeny derived therefrom without regard to the number of passages.

The present invention also relates to a coronavirus spike protein according to the invention or a nucleic acid molecule according to the invention for use as a vaccine.

The present invention further relates to a coronavirus spike protein according to the invention or a nucleic acid molecule according to the invention for use in prevention or treatment of a coronavirus infection or coronavirus infectious disease.

In an embodiment, the coronavirus infection is an infection caused by a coronavirus that is selected from the group consisting of MERS-CoV, SARS-CoV and SARS-CoV-2, preferably SARS-CoV-2. The coronavirus infection may cause a coronavirus infectious disease in a subject, preferably a human subject. In an embodiment, the coronavirus infectious disease is selected from the group consisting of MERS for MERS-CoV, SARS for SARS-CoV and COVID-19 for SARS-CoV-2.

A related aspect of the invention defines a method for preventing or treating a coronavirus infection or a coronavirus infectious disease. The method comprises administering an effective amount of the coronavirus spike protein or nucleic acid molecule according to the invention to a subject suffering from a coronavirus infection or infectious disease or having a risk of suffering from a coronavirus infection or infectious disease.

Treatment of a coronavirus infection or infectious disease as used herein does not necessarily mean curative treatment of the coronavirus infection or infectious disease but also encompasses inhibition or reduction of the short- and long-term symptoms of the coronavirus infection or infectious disease.

Hence, treatment also encompasses delaying onset of the coronavirus infection or infectious disease, including delaying, preventing onset of symptoms or resolving established pathologies associated with the coronavirus infection or infectious disease.

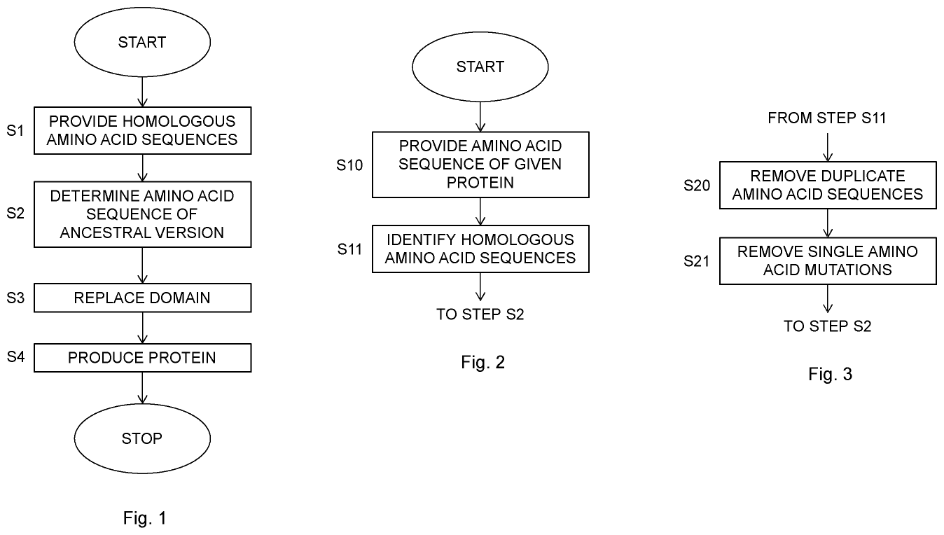

Another aspect of the invention relates to a protein production method, see FIG. 1. The method comprises providing, in step S1, a plurality of homologous amino acid sequences of a given protein. The method also comprises determining, in step S2, an amino acid sequence of an ancestral version of the given protein in an ancestral sequence reconstruction method based on the plurality of homologous amino acid sequences of the given protein. The method further comprises replacing, in step S3, a domain of the amino acid sequence of the ancestral version of the given protein with a corresponding domain derived from an amino acid sequence of the given protein or a homologous version thereof. The method additionally comprises producing, in step S4, a protein comprising the amino acid sequence obtained by replacing the domain of the amino acid sequence of the ancestral version of the given protein with the corresponding domain derived from the amino acid sequence of the given protein or the homologous version thereof.

The method of the invention thereby uses an ancestral sequence reconstruction method to determine an amino acid sequence of a protein based on a plurality of homologous amino acid sequences of a given protein, also referred herein as starting protein or input protein. “Homologous” amino acid sequences as used herein indicate that the amino acid sequences share sequence similarity and have a common ancestor.

In an embodiment, step S3 comprises replacing a domain of the amino acid sequence of the ancestral version of the given protein with a corresponding domain derived from an amino acid sequence selected among the plurality of homologous amino acid sequences of the given protein. In this embodiment, step S4 comprises producing a protein comprising the amino acid sequence obtained by replacing the domain of the amino acid sequence of the ancestral version of the given protein with the corresponding domain derived from the amino acid sequence selected among the plurality of homologous amino acid sequences of the given protein.

A homologous version of the given protein as used herein is, thus, preferably selected among the plurality of homologous amino acid sequences of the given protein provided in step S1. The embodiments are, however, not limited thereof. The homologous version of the given protein could also be another homologous amino acid sequence of the given protein that is not among the plurality of homologous amino acid sequences as provided in step S1.

In another embodiment, step S3 comprises replacing a domain of the amino acid sequence of the ancestral version of the given protein with a corresponding domain derived from an amino acid sequence of the given protein. In this embodiment, step S4 comprises producing a protein comprising the amino acid sequence obtained by replacing the domain of the amino acid sequence of the ancestral version of the given protein with the corresponding domain derived from the amino acid sequence of the given protein.

The amino acid sequence of the ancestral version of the given protein as obtained from the ancestral sequence reconstruction method in step S2 is then modified in step S3 by replacing at least one domain of the amino acid sequence determined in step S2 with a respective corresponding domain derived from one or more of the amino acid sequences provided in step S1, i.e., of the homologous amino acid sequences of the given protein. The protein as produced in step S4 therefore comprises the amino acid sequence obtained by replacing the at least one domain of the amino acid sequence of the ancestral version of the given protein with the corresponding respective domain derived from an amino acid sequence selected among the plurality of homologous amino acid sequences of the given protein. This means that the protein comprises a major portion that corresponds to the ancestral version of the given protein but with at least one domain from one of the homologous amino acid sequences of the given protein. In other words, the protein comprises one or more amino acid domains, i.e., at least one or more protein domains, from the ancestral version of the given protein and one or more amino acid domains, i.e., at least one or more protein domains, from a currently existing version of the given protein, as represented by the plurality of homologous amino acid sequences provided in step S1.

The mixture of the amino acid or protein domains in the protein as produced in step S4 provides significant advantages to the protein in terms of being useful as antigen candidate or in vaccine production. Firstly, the amino acid or protein domain(s) of the protein originating from the ancestral version of the given protein as determined in step S2 provides the previously mentioned advantages of ancestral proteins including inherent robustness and enables production at high titers. Secondly, the amino acid or protein domain(s) of the protein as taken from one or more of the homologous amino acid sequences of the given protein means that the protein comprises at least one portion or domain that corresponds to a currently existing version of the given protein. This at least one portion or domain could, for instance, correspond to a domain of a pathogen protein that is configured to interact with a host protein or receptor during pathogen infection of a host subject. For instance, this at least one portion or domain could be involved in the initial attachment of the pathogen to a cell of a subject and/or penetration through or fusion with the cellular membrane of the cell or through another cell entry mechanism, such as endocytosis. This at least one protein portion or domain is thereby adapted to infect cells using currently existing and future versions of receptors or molecules attached to or anchored in the cell membranes.

Hence, illustrative examples of domains that could be replaced in step S3 include receptor binding domains and host binding domains of the ancestral version of the given protein.

A single domain of the amino acid sequence of the ancestral version of the given protein could be replaced in step S3 or multiple domains of the of the amino acid sequence of the ancestral version of the given protein could be replaced in step S3.

A protein consisting of at least one amino acid sequence and protein domain derived from the ancestral version of the given protein and at least one amino acid sequence and protein domain derived from the given protein or the homologous version thereof, such as one or more of the homologous amino acid sequences provided in step S1, generally has improved characteristics as compared to the given protein and the ancestral version of the given protein determined in step S2. The protein produced in step S4 is typically more robust and can be produced in higher titers as compared to the given protein. Furthermore, the protein produced in step S4 is more antigenic as compared to the ancestral version of the given protein determined in step S2 in terms of antibodies raised by a subject against the protein produced in step S4 which are believed to be more effective in protecting the subject against current and emerging strains of a pathogen as compared to the ancestral version of the given protein determined in step S2 when the given protein is a pathogen protein, i.e., a protein from a pathogen.

FIG. 2 is a flow chart illustrating various embodiments of step S1. In these various embodiments, the method starts in step S10, which comprises providing an amino acid sequence of the given protein. The method then continues to step S11. This step S11 comprises identifying a plurality of amino acid sequences. The method then continues to step S2 in FIG. 1.

In an embodiment, step S11 comprises identifying a plurality of amino acid sequences having a sequence identity of at least 40% with the provided amino acid sequence of the given protein. In preferred embodiments, step S11 comprises identifying a plurality of amino acid sequences having a sequence identity of at least 50% with the provided amino acid sequence of the given protein, preferably at least 60% with the provided amino acid sequence of the given protein and more preferably at least 70% with the provided amino acid sequence of the given protein.

In another embodiment, step S11 comprises identifying, in a protein database, the N amino acid sequences having highest sequence identity with the provided amino acid sequence of the given protein. The parameter N is at least 25. In preferred embodiments, the parameter N is at least 50, more preferably at least 100, and even more preferably at least 200.

Hence, in these embodiments, an initial amino acid sequence of the given protein is provided in step S10 and used as input to search for homologous amino acid sequences of the given protein, such as in one or more protein or amino acid databases. For instance, the amino acid sequence provided in step S10 could be used as input sequence for a BLAST search for homologous sequences.

The various embodiments of step S11 then identify either a minimum number of homologous amino acid sequences or amino acid sequences having a minimum sequence identity with the provided amino acid sequence.

In an embodiment, the method comprises an optional, but preferred step S20 as shown in FIG. 3. In such a case, the method continues from step S11 in FIG. 2. This step S20 comprises removing, from the identified amino acid sequences, any duplicate amino acid sequences. The method then continues to step S2 or to the optional step S21.

Hence, in this embodiment, duplicate amino acid sequences are removed so that the homologous amino acid sequences input to the ancestral sequence reconstruction method in step S2 only comprises one copy of each unique amino acid sequence. This step S20 reduces the risk of putting biases onto any amino acid sequence of the given protein that is present as multiple identical versions or copies in the protein database.

FIG. 3 also illustrates another optional, but preferred step S21 of the method. This step S21 comprises removing, from the identified amino acid sequences, any amino acid sequence being a single amino acid mutant of the amino acid sequence of the given protein. This step S21 also reduces the risk of putting biases onto near identical amino acid sequences of the given protein. Step S21 is optional and may be omitted. In such an embodiment, also single amino acid mutants of the amino acid sequence of the given protein are input into the ancestral sequence reconstruction method in step S2.

In an embodiment, the method comprises step S20. In another embodiment, the method comprises step S21. In a further embodiment, the method comprises steps S20 and S21. In this latter embodiment, steps S20 and S21 can be performed serially in any order or at least partly in parallel.

In an embodiment, step S3 in FIG. 1 comprises replacing the domain of the amino acid sequence of the ancestral version of the given protein with a corresponding domain derived from the provided amino acid sequence of the given protein. Hence, in this particular embodiment, step S3 comprises replacing the domain of the amino acid sequence of the ancestral version of the given protein with a corresponding domain derived from the amino acid sequence provided in step S10.

In another embodiment, step S3 in FIG. 1 comprises replacing the domain of the amino acid sequence of the ancestral version of the given protein with a corresponding domain derived from one of the amino acid sequences identified in step S11.

In an embodiment, step S2 comprises determining the amino acid sequence of a node of a phylogenetic tree generated in the ancestral sequence reconstruction method based on the plurality of homologous amino acid sequences of the given protein.

For instance, a sequence identity search could be conducted in a protein database based on the amino acid sequence of a given extant protein. The top hits ranging, for instance, from 100-1000 sequences are used to construct an initial phylogenetic tree. The sequences are then selected and scrutinized for relevance and similarity in the phylogenetic tree, including evolutionary relevance and uniqueness of amino acid sequences and optionally excluding single amino acid mutations of a protein sequence from the selection of amino acid sequences. A sub-selection of the initial sequences could be used to perform a multiple sequence alignment. The multiple sequence alignment may be optionally trimmed, e.g., by removing regions to harmonize sequence length. Based on the multiple sequence alignment, the phylogenetic tree is constructed with a suitable ancestral sequence reconstruction algorithm or software, such as IQ-Tree. An amino acid substitution matrix (also referred to as the “evolutionary model”) that results in a phylogenetic tree with the highest likelihood is selected via the ancestral sequence reconstruction algorithm or software to determine the most likely evolutionary trajectory. The phylogenetic tree is then analyzed with the alignment for most likely substitutions on every ancestral node, creating the inferred ancestral sequence.

In an embodiment, step S3 comprises replacing a receptor binding domain of the amino acid sequence of the ancestral version of the given protein with a corresponding receptor binding domain derived from the amino acid sequence of the given protein or the homologous version thereof.

In another embodiment, step S3 comprises replacing a host binding domain of the amino acid sequence of the ancestral version of the given protein with a corresponding host binding domain derived from the amino acid sequence of the given protein or the homologous version thereof. In this embodiment, the host binding domain is configured to bind to a macromolecule present on a cell surface of an animal cell, preferably a mammalian cell, and more preferably a human cell.

In a further embodiment, step S3 comprises replacing an antigenic or immunogenic domain of the amino acid sequence of the ancestral version of the given protein with a corresponding antigenic or immunogenic domain derived from the amino acid sequence of the given protein or the homologous version thereof. Immunogenic domain as used herein refers to a domain, sometimes also referred to as an immunogenic site, of a protein having the ability to induce a cellular and humoral immune response in a host. Correspondingly, antigenic domain as used herein refers to a domain, sometimes also referred to as antigenic site, of a protein having the ability to be specifically recognized by antibodies generated as a result of an immune response to the given domain.

In a particular embodiment, step S3 comprises replacing an antigenic domain of the amino acid sequence of the ancestral version of the given protein with a corresponding antigenic domain derived from the amino acid sequence of the given protein or the homologous version thereof. In another particular embodiment, step S3 comprises replacing an immunogenic domain of the amino acid sequence of the ancestral version of the given protein with a corresponding immunogenic domain derived from the amino acid sequence of the given protein or the homologous version thereof.

Hence, in an embodiment, step S3 comprises replacing a receptor binding domain, a host binding domain, an antigenic domain or an immunogenic domain of the amino acid sequence of the ancestral version of the given protein with a corresponding receptor binding domain, a corresponding host binding domain, a corresponding antigenic domain or a corresponding immunogenic domain derived from the amino acid sequence of the given protein or the homologous version thereof.

In a particular embodiment, step S3 comprises replacing a receptor binding domain or a host binding domain of the amino acid sequence of the ancestral version of the given protein with a corresponding receptor binding domain or a corresponding host binding domain derived from the amino acid sequence of the given protein or the homologous version thereof.

In an embodiment, the domain of the amino acid sequence of the ancestral version of the given protein is a domain of a plurality of M consecutive amino acids of the amino acid sequence of the ancestral version of the given protein. In this embodiment, the corresponding domain derived from the amino acid sequence of the given protein or the homologous version thereof is a corresponding domain of a plurality of N consecutive amino acids of the amino acid sequence of the given protein or the homologous version thereof. Each of M, N is at least 5, preferably at least 10, and more preferably at least 25. N could be equal to or different than M, such as larger than M or smaller than M.

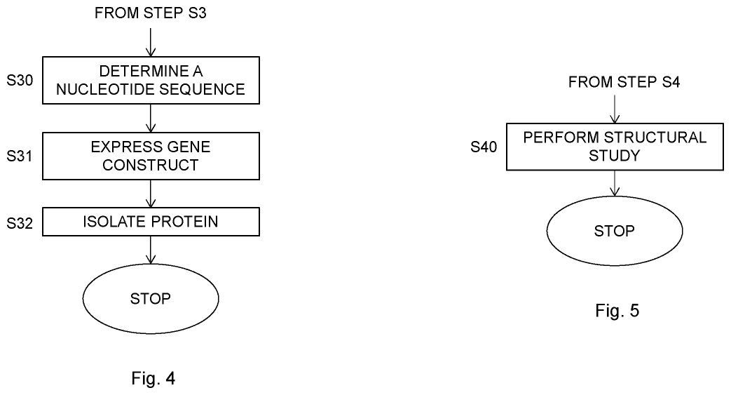

FIG. 4 is a flow chart illustrating an embodiment of step S4 in FIG. 1. In this embodiment, the method continues from step S3 in FIG. 1. A next step S30 comprises determining a nucleotide sequence encoding the amino acid sequence obtained by replacing the domain of the amino acid sequence of the ancestral version of the given protein with the corresponding domain derived from the amino acid sequence of the given protein or the homologous version thereof. The following step S31 comprises expressing a gene construct comprising the determined nucleotide sequence in a host cell comprising the gene construct and isolating, in step S32, the protein from the host cell or from a culture medium, in which the host cell is cultured.

Thus, the amino acid sequence of the protein is used as a basis for determining the nucleotide sequence in step S30 to get a nucleic acid sequence encoding the protein. This nucleic acid sequence is optionally codon-optimized for expression in a selected host cell. A gene construct comprising the determined nucleic acid sequence, such as in the form of an expression vector comprising the determined nucleic acid sequence, is expressed in a selected host cell, such as a prokaryotic host cell, e.g., a bacterial host cell, or an eukaryotic host cell, e.g., a yeast cell, a mammalian cell or a human cell, to produce the protein. The produced protein is then isolated from the host cell in step S32 and/or from the culture medium if the protein is secreted into the culture medium.

The isolating step S32 is conducted according to well-known protein isolation or purification protocols, such as disclosed in the Example section.

FIG. 5 is a flow chart illustrating an additional, optional step of the method. The method continues from step S4 in FIG. 1 to step S40. This step S40 comprises performing a structural study of the protein produced in step S4.

Various types of structural studies can be performed in step S40 including, but not limited to, X-ray crystallography and cryo-electron (CE) microscopy. In a currently preferred embodiment, step S40 comprises performing structural study of the protein produced in step S4 by CE microscopy.

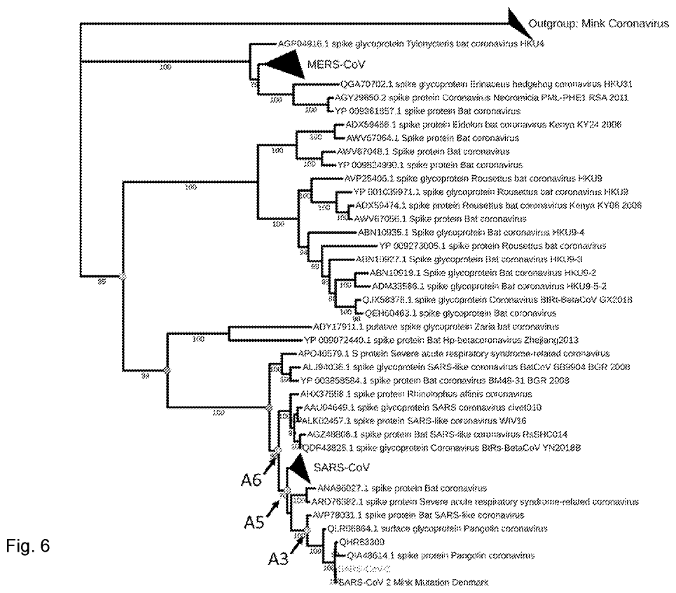

FIG. 6 illustrates a phylogenic tree obtained for various spike proteins. The phylogenic tree further indicates coronavirus spike proteins according to the embodiments (A3, A5, A6).

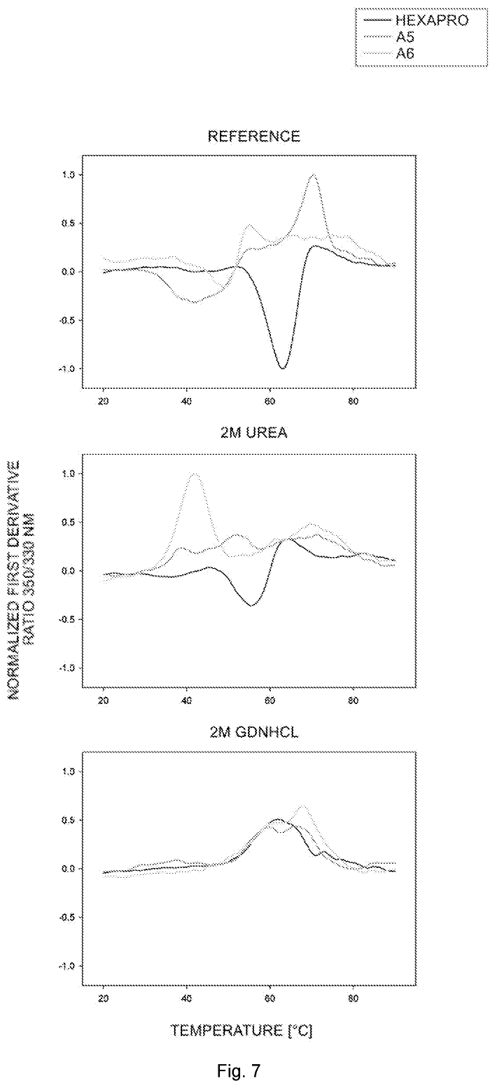

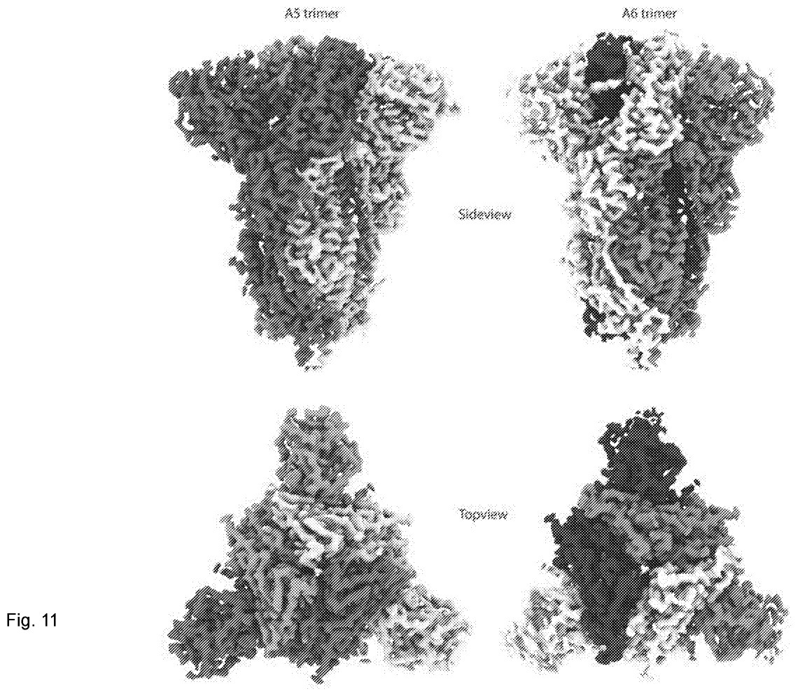

The protein as produced in step S4 is, as is further described herein, robust and can be produced in high titer and yield as shown in FIGS. 7 to 9. Accordingly, the protein is suitable for structural studies, in which these properties are of benefit as shown in FIG. 11. The structural studies conducted in step S40 could provide structural insight into the 3D structure of the given protein even though structural studies might not be possible to perform on the given protein itself due to low robustness and low titer. The protein as produced in step S40 then constitutes a structural substitute for the given protein.

In an embodiment, the given protein is a protein of a pathogen, also referred to as pathogen protein herein, which produces a disease in animals, preferably mammals and more preferably humans. In an embodiment, the pathogen is selected from the group consisting of bacteria, fungi, prions, viroids, viruses and protozoans. In a preferred embodiment, the pathogen is selected from the group consisting of bacteria, fungi and viruses. In a currently preferred embodiment, the pathogen is a virus and the given protein is a viral or virus protein.

In an embodiment, step S1 in FIG. 1 comprises providing a plurality of homologous amino acid sequences of a pathogen protein. In this embodiment, step S2 comprises determining an amino acid sequence of an ancestral version of the pathogen protein in an ancestral sequence reconstruction method based on the plurality of homologous amino acid sequences of the pathogen protein. Step S3 comprises, in this embodiment, replacing a domain of the amino acid sequence of the ancestral version of the pathogen protein with a corresponding domain derived from an amino acid sequence of the pathogen protein or a homologous version thereof. In this embodiment, step S4 comprises producing an antigenic protein comprising the amino acid sequence obtained by replacing the domain of the amino acid sequence of the ancestral pathogen protein with the corresponding domain derived from the amino acid sequence of the pathogen protein or the homologous version thereof.

The present invention also relates to a protein obtainable by the method according to the invention, such as shown in any of FIGS. 1 to 5.

wt SARS-CoV-2 spike protein
(SEQ ID NO: 1)
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNFRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPR
RARSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIANQFNSA
IGKIQDSLSSTASALGKLQDVVNQNAQALNTLVKQLSSNFGAISSVLNDILSRLDKVEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQYIKWPWYIWLGFIAGLIAIVMVTIMLCCMTSC
CSCLKGCCSCGSCCKFDEDDSEPVLKGVKLHYT

The coronavirus spike proteins presented below have the following general formula: SP-Seq1-RBD-Seq2-GS-T4-GTS-HRV 3C-G-HIS-Strep.

A3
(SEQ ID NO: 2)
MFVFLVLLPLVSSQCVNLTGRTPLNPNYTNSSQRGVYYPDTIFRSDTLVLSQGYFLPFYSNV
SWYYSLTTNNAATKRFDNPILDFKDGIYFAATEKSNIIRGWIFGTTLDNTSQSLLIVNNATN
VIIKVCNFQFCYDPYLSGYYGHNNKTWSIREFAVYSSYANCTFEYVSKSFMLDISGKGGLFN
TLREFVFRNVDGYFKIYSKYTPVNLNRGLPTGFSVLQPLVELPVGINITKERTLLTIHRGDP
MPNNGWTAFSAAYFVGYLKPRTFMLKYNENGTITDAVDCALDPLSETKCTLKSLTVQKGIYQ
TSNFRVQPTQSVVRFPNITNLCPFHKVFNATRFPSVYAWERTKISDCVADYTVFYNSTSFST
FKCYGVSPSKLIDLCFTSVYADTFLIRFSEVRQVAPGQTGVIADYNYKLPDDFTGCVIAWNT
AKQDVGNYFYRSHRSTKLKPFERDLSSQDENGVRTLSTYDFNPNVPLEYQATRVVVLSFELL
NAPATVCGPKLSTQLVKNQCVNFNFNGLKGTGVLTDSSKRFQSFQQFGRDASDFTDSVRDPQ
TLEILDITPCSFGGVSVITPGTNTSSEVAVLYQDVNCTDVPTAIHADQLTPAWRIYSTGTNV
FQTQAGCLIGAEHVNASYECDIPIGAGICASYHTASILRSTSQKAIVAYTMSLGAENSIAYA
NNSIAIPTNFSISVTTEVMPVSMAKTSVDCTMYICGDSIECSNLLLQYGSFCTQLNRALTGI
AIEQDKNTQEVFAQVKQIYKTPPIKDFGGFNFSQILPDPSKPSKRSFIEDLLENKVTLADAG
FIKQYGDCLGDISARDLICAQKFNGLTVLPPLLTDEMIAAYTAALISGTATAGWTFGAGAAL
QIPFAMQMAYRENGIGVTQNVLYENQKLIANQFNSAIGKIQESLTSTASALGKLQDVVNQNA
QALNTLVKQLSSNFGAISSVLNDILSRLDKVEAEVQIDRLITGRLQSLQTYVTQQLIRAAEI
RASANLAATKMSECVLGQSKRVDFCGKGYHLMSFPQSAPHGVVFLHVTYVPSQEKNFTTAPA
ICHEGKAHFPREGVFVSNGTHWFVTQRNFYEPQIITTDNTFVSGNCDVVIGIINNTVYDPLQ
PELDSFKEELDKYFKNHTSPDVDLGDISGINASVVNIQKEIDRLNEVAKNLNESLIDLQELG
KYEQGSGYIPEAPRDGQAYVRKDGEWVLLSTFLGTSLEVLFQGPGHHHHHHHHSAWSHPQFE
KGGGSGGGGSGGSAWSHPQFEK
A3 without C-terminal additions
(SEQ ID NO: 3)
MFVFLVLLPLVSSQCVNLTGRTPLNPNYTNSSQRGVYYPDTIFRSDTLVLSQGYFLPFYSNV
SWYYSLTTNNAATKRFDNPILDFKDGIYFAATEKSNIIRGWIFGTTLDNTSQSLLIVNNATN
VIIKVCNFQFCYDPYLSGYYGHNNKTWSIREFAVYSSYANCTFEYVSKSFMLDISGKGGLFN
TLREFVFRNVDGYFKIYSKYTPVNLNRGLPTGFSVLQPLVELPVGINITKERTLLTIHRGDP
MPNNGWTAFSAAYFVGYLKPRTFMLKYNENGTITDAVDCALDPLSETKCTLKSLTVQKGIYQ
TSNFRVQPTQSVVRFPNITNLCPFHKVFNATRFPSVYAWERTKISDCVADYTVFYNSTSFST
FKCYGVSPSKLIDLCFTSVYADTFLIRFSEVRQVAPGQTGVIADYNYKLPDDFTGCVIAWNT
AKQDVGNYFYRSHRSTKLKPFERDLSSQDENGVRTLSTYDFNPNVPLEYQATRVVVLSFELL
NAPATVCGPKLSTQLVKNQCVNFNFNGLKGTGVLTDSSKRFQSFQQFGRDASDFTDSVRDPQ
TLEILDITPCSFGGVSVITPGTNTSSEVAVLYQDVNCTDVPTAIHADQLTPAWRIYSTGTNV
FQTQAGCLIGAEHVNASYECDIPIGAGICASYHTASILRSTSQKAIVAYTMSLGAENSIAYA
NNSIAIPTNFSISVTTEVMPVSMAKTSVDCTMYICGDSIECSNLLLQYGSFCTQLNRALTGI
AIEQDKNTQEVFAQVKQIYKTPPIKDFGGFNFSQILPDPSKPSKRSFIEDLLFNKVTLADAG
FIKQYGDCLGDISARDLICAQKFNGLTVLPPLLTDEMIAAYTAALISGTATAGWTFGAGAAL
QIPFAMQMAYRFNGIGVTQNVLYENQKLIANQFNSAIGKIQESLTSTASALGKLQDVVNQNA
QALNTLVKQLSSNFGAISSVLNDILSRLDKVEAEVQIDRLITGRLQSLQTYVTQQLIRAAEI
RASANLAATKMSECVLGQSKRVDFCGKGYHLMSFPQSAPHGVVFLHVTYVPSQEKNFTTAPA
ICHEGKAHFPREGVFVSNGTHWFVTQRNFYEPQIITTDNTFVSGNCDVVIGIINNTVYDPLQ
PELDSFKEELDKYFKNHTSPDVDLGDISGINASVVNIQKEIDRLNEVAKNLNESLIDLQELG
KYEQ
A3 without N-terminal signal peptide and C-terminal additions
(SEQ ID NO: 4)
QCVNLTGRTPLNPNYTNSSQRGVYYPDTIFRSDTLVLSQGYFLPFYSNVSWYYSLTINNAAT
KRFDNPILDFKDGIYFAATEKSNIIRGWIFGTTLDNTSQSLLIVNNATNVIIKVCNFQFCYD
PYLSGYYGHNNKTWSIREFAVYSSYANCTFEYVSKSFMLDISGKGGLFNTLREFVFRNVDGY
FKIYSKYTPVNLNRGLPTGFSVLQPLVELPVGINITKERTLLTIHRGDPMPNNGWTAFSAAY
FVGYLKPRTFMLKYNENGTITDAVDCALDPLSETKCTLKSLTVQKGIYQTSNERVQPTQSVV
RFPNITNLCPFHKVFNATRFPSVYAWERTKISDCVADYTVFYNSTSFSTFKCYGVSPSKLID
LCFTSVYADTFLIRFSEVRQVAPGQTGVIADYNYKLPDDFTGCVIAWNTAKQDVGNYFYRSH
RSTKLKPFERDLSSQDENGVRTLSTYDFNPNVPLEYQATRVVVLSFELLNAPATVCGPKLST
QLVKNQCVNFNFNGLKGTGVLTDSSKRFQSFQQFGRDASDFTDSVRDPQTLEILDITPCSFG
GVSVITPGTNTSSEVAVLYQDVNCTDVPTAIHADQLTPAWRIYSTGTNVFQTQAGCLIGAEH
VNASYECDIPIGAGICASYHTASILRSTSQKAIVAYTMSLGAENSIAYANNSIAIPTNFSIS
VTTEVMPVSMAKTSVDCTMYICGDSIECSNLLLQYGSFCTQLNRALTGIAIEQDKNTQEVFA
QVKQIYKTPPIKDFGGFNFSQILPDPSKPSKRSFIEDLLFNKVTLADAGFIKQYGDCLGDIS
ARDLICAQKFNGLTVLPPLLTDEMIAAYTAALISGTATAGWTFGAGAALQIPFAMQMAYRFN
GIGVTQNVLYENQKLIANQFNSAIGKIQESLTSTASALGKLQDVVNQNAQALNTLVKQLSSN
FGAISSVLNDILSRLDKVEAEVQIDRLITGRLQSLQTYVTQQLIRAAEIRASANLAATKMSE
CVLGQSKRVDFCGKGYHLMSFPQSAPHGVVFLHVTYVPSQEKNFTTAPAICHEGKAHFPREG
VFVSNGTHWFVTQRNFYEPQIITTDNTFVSGNCDVVIGIINNTVYDPLQPELDSFKEELDKY
FKNHTSPDVDLGDISGINASVVNIQKEIDRLNEVAKNLNESLIDLQELGKYEQ
A3-part 1 without N-terminal signal peptide
(SEQ ID NO: 5)
QCVNLTGRTPLNPNYTNSSQRGVYYPDTIFRSDTLVLSQGYFLPFYSNVSWYYSLTINNAAT
KRFDNPILDFKDGIYFAATEKSNIIRGWIFGTTLDNTSQSLLIVNNATNVIIKVCNFQFCYD
PYLSGYYGHNNKTWSIREFAVYSSYANCTFEYVSKSFMLDISGKGGLFNTLREFVFRNVDGY
FKIYSKYTPVNLNRGLPTGFSVLQPLVELPVGINITKERTLLTIHRGDPMPNNGWTAFSAAY
FVGYLKPRTFMLKYNENGTITDAVDCALDPLSETKCTLKSLTVQKGIYQTSNF
A3-part 1
(SEQ ID NO: 6)
MFVFLVLLPLVSSQCVNLTGRTPLNPNYTNSSQRGVYYPDTIFRSDTLVLSQGYFLPFYSNV
SWYYSLTTNNAATKRFDNPILDFKDGIYFAATEKSNIIRGWIFGTTLDNTSQSLLIVNNATN
VIIKVCNFQFCYDPYLSGYYGHNNKTWSIREFAVYSSYANCTFEYVSKSFMLDISGKGGLFN
TLREFVFRNVDGYFKIYSKYTPVNLNRGLPTGFSVLQPLVELPVGINITKERTLLTIHRGDP
MPNNGWTAFSAAYFVGYLKPRTFMLKYNENGTITDAVDCALDPLSETKCTLKSLTVQKGIYQ
TSNF
A3-RBD
(SEQ ID NO: 7)
RVQPTQSVVRFPNITNLCPFHKVFNATRFPSVYAWERTKISDCVADYTVFYNSTSFSTEKCY
GVSPSKLIDLCFTSVYADTFLIRFSEVRQVAPGQTGVIADYNYKLPDDFTGCVIAWNTAKQD
VGNYFYRSHRSTKLKPFERDLSSQDENGVRTLSTYDENPNVPLEYQATRVVVLSFELLNAPA
TVCGPKLSTQLVKNQCVNF
A3-part 2
(SEQ ID NO: 8)
NFNGLKGTGVLTDSSKRFQSFQQFGRDASDFTDSVRDPQTLEILDITPCSFGGVSVITPGTN
TSSEVAVLYQDVNCTDVPTAIHADQLTPAWRIYSTGTNVFQTQAGCLIGAEHVNASYECDIP
IGAGICASYHTASILRSTSQKAIVAYTMSLGAENSIAYANNSIAIPTNFSISVTTEVMPVSM
AKTSVDCTMYICGDSIECSNLLLQYGSFCTQLNRALTGIAIEQDKNTQEVFAQVKQIYKTPP
IKDFGGFNFSQILPDPSKPSKRSFIEDLLENKVTLADAGFIKQYGDCLGDISARDLICAQKF
NGLTVLPPLLTDEMIAAYTAALISGTATAGWTFGAGAALQIPFAMQMAYRFNGIGVTQNVLY
ENQKLIANQFNSAIGKIQESLTSTASALGKLQDVVNQNAQALNTLVKQLSSNFGAISSVIND
ILSRLDKVEAEVQIDRLITGRLQSLQTYVTQQLIRAAEIRASANLAATKMSECVLGQSKRVD
FCGKGYHLMSFPQSAPHGVVFLHVTYVPSQEKNFTTAPAICHEGKAHFPREGVFVSNGTHWF
VTQRNFYEPQIITTDNTFVSGNCDVVIGIINNTVYDPLQPELDSFKEELDKYFKNHTSPDVD
LGDISGINASVVNIQKEIDRLNEVAKNLNESLIDLQELGKYEQ
A3 with wt RBD
(SEQ ID NO: 9)
MFVFLVLLPLVSSQCVNLTGRTPLNPNYTNSSQRGVYYPDTIFRSDTLVLSQGYFLPFYSNV
SWYYSLTTNNAATKRFDNPILDFKDGIYFAATEKSNIIRGWIFGTTLDNTSQSLLIVNNATN
VIIKVCNFQFCYDPYLSGYYGHNNKTWSIREFAVYSSYANCTFEYVSKSFMLDISGKGGLFN
TLREFVFRNVDGYFKIYSKYTPVNLNRGLPTGFSVLQPLVELPVGINITKERTLLTIHRGDP
MPNNGWTAFSAAYFVGYLKPRTFMLKYNENGTITDAVDCALDPLSETKCTLKSLTVQKGIYQ
TSNFRVQPTESIVRFPNITNLCPFGEVFNATRFASVYAWNRKRISNCVADYSVLYNSASFST
FKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLKGTGVLTDSSKRFQSF
QQFGRDASDFTDSVRDPQTLEILDITPCSFGGVSVITPGTNTSSEVAVLYQDVNCTDVPTAI
HADQLTPAWRIYSTGTNVFQTQAGCLIGAEHVNASYECDIPIGAGICASYHTASILRSTSQK
AIVAYTMSLGAENSIAYANNSIAIPTNFSISVTTEVMPVSMAKTSVDCTMYICGDSIECSNL
LLQYGSFCTQLNRALTGIAIEQDKNTQEVFAQVKQIYKTPPIKDFGGFNFSQILPDPSKPSK
RSFIEDLLFNKVTLADAGFIKQYGDCLGDISARDLICAQKFNGLTVLPPLLTDEMIAAYTAA
LISGTATAGWTFGAGAALQIPFAMQMAYRFNGIGVTQNVLYENQKLIANQFNSAIGKIQESL
TSTASALGKLQDVVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLMSFPQSAPHGVVF
LHVTYVPSQEKNFTTAPAICHEGKAHFPREGVFVSNGTHWFVTQRNFYEPQIITTDNTFVSG
NCDVVIGIINNTVYDPLQPELDSFKEELDKYFKNHTSPDVDLGDISGINASVVNIQKEIDRL
NEVAKNLNESLIDLQELGKYEQGSGYIPEAPRDGQAYVRKDGEWVLLSTFLGTSLEVLFQGP
GHHHHHHHHSAWSHPQFEKGGGSGGGGSGGSAWSHPQFEK
A3 with wt RBD without C-terminal additions
(SEQ ID NO: 10)
MFVFLVLLPLVSSQCVNLTGRTPLNPNYTNSSQRGVYYPDTIFRSDTLVLSQGYFLPFYSNV
SWYYSLTTNNAATKRFDNPILDFKDGIYFAATEKSNIIRGWIFGTTLDNTSQSLLIVNNATN
VIIKVCNFQFCYDPYLSGYYGHNNKTWSIREFAVYSSYANCTFEYVSKSFMLDISGKGGLFN
TLREFVFRNVDGYFKIYSKYTPVNLNRGLPTGFSVLQPLVELPVGINITKFRTLLTIHRGDP
MPNNGWTAFSAAYFVGYLKPRTFMLKYNENGTITDAVDCALDPLSETKCTLKSLTVQKGIYQ
TSNFRVQPTESIVRFPNITNLCPFGEVFNATRFASVYAWNRKRISNCVADYSVLYNSASFST
FKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLKGTGVLTDSSKRFQSF
QQFGRDASDFTDSVRDPQTLEILDITPCSFGGVSVITPGTNTSSEVAVLYQDVNCTDVPTAI
HADQLTPAWRIYSTGTNVFQTQAGCLIGAEHVNASYECDIPIGAGICASYHTASILRSTSQK
AIVAYTMSLGAENSIAYANNSIAIPTNFSISVTTEVMPVSMAKTSVDCTMYICGDSIECSNL
LLQYGSFCTQLNRALTGIAIEQDKNTQEVFAQVKQIYKTPPIKDFGGFNFSQILPDPSKPSK
RSFIEDLLFNKVTLADAGFIKQYGDCLGDISARDLICAQKFNGLTVLPPLLTDEMIAAYTAA
LISGTATAGWTFGAGAALQIPFAMQMAYRFNGIGVTQNVLYENQKLIANQFNSAIGKIQESL
TSTASALGKLQDVVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLMSFPQSAPHGVVF
LHVTYVPSQEKNFTTAPAICHEGKAHFPREGVFVSNGTHWFVTQRNFYEPQIITTDNTFVSG
NCDVVIGIINNTVYDPLQPELDSFKEELDKYFKNHTSPDVDLGDISGINASVVNIQKEIDRL
NEVAKNLNESLIDLQELGKYEQ
A3 with wt RBD without N-terminal signal peptide and C-terminal
additions
(SEQ ID NO: 11)
QCVNLTGRTPLNPNYTNSSQRGVYYPDTIFRSDTLVLSQGYFLPFYSNVSWYYSLTTNNAAT
KRFDNPILDFKDGIYFAATEKSNIIRGWIFGTTLDNTSQSLLIVNNATNVIIKVCNFQFCYD
PYLSGYYGHNNKTWSIREFAVYSSYANCTFEYVSKSFMLDISGKGGLFNTLREFVFRNVDGY
FKIYSKYTPVNLNRGLPTGFSVLQPLVELPVGINITKERTLLTIHRGDPMPNNGWTAFSAAY
FVGYLKPRTFMLKYNENGTITDAVDCALDPLSETKCTLKSLTVQKGIYQTSNERVQPTESIV
RFPNITNLCPFGEVFNATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGNYNY
LYRLFRKSNLKPFERDISTEIYQAGSTPCNGVEGFNCYFPLQSYGFQPTNGVGYQPYRVVVL
SFELLHAPATVCGPKKSTNLVKNKCVNFNFNGLKGTGVLTDSSKRFQSFQQFGRDASDFTDS
VRDPQTLEILDITPCSFGGVSVITPGTNTSSEVAVLYQDVNCTDVPTAIHADQLTPAWRIYS
TGTNVFQTQAGCLIGAEHVNASYECDIPIGAGICASYHTASILRSTSQKAIVAYTMSLGAEN
SIAYANNSIAIPTNFSISVTTEVMPVSMAKTSVDCTMYICGDSIECSNLLLQYGSFCTQLNR
ALTGIAIEQDKNTQEVFAQVKQIYKTPPIKDFGGFNFSQILPDPSKPSKRSFIEDLLFNKVT
LADAGFIKQYGDCLGDISARDLICAQKFNGLTVLPPLLTDEMIAAYTAALISGTATAGWTFG
AGAALQIPFAMQMAYRFNGIGVTQNVLYENQKLIANQFNSAIGKIQESLTSTASALGKLQDV
VNQNAQALNTLVKQLSSNFGAISSVLNDILSRLDKVEAEVQIDRLITGRLQSLQTYVTQQLI
RAAEIRASANLAATKMSECVLGQSKRVDFCGKGYHLMSFPQSAPHGVVFLHVTYVPSQEKNF
TTAPAICHEGKAHFPREGVFVSNGTHWFVTQRNFYEPQIITTDNTFVSGNCDVVIGIINNTV
YDPLQPELDSFKEELDKYFKNHTSPDVDLGDISGINASVVNIQKEIDRLNEVAKNLNESLID
LQELGKYEQ
A5
(SEQ ID NO: 12)
MFVFLVLLPLVSSTAQEGTCGTISNKTPPNMNQFSSSRRGVYYPDDIFRSDVLHLTQDYFLP
FNSNVTRYLSLNADSNRIVRFDNPILPEGDGIYFAATEKSNVIRGWIFGSTLDNTSQSAIIV
NNSTHIIIKVCNFQLCDDPMFTVSRGQHYKTWVYTNARNCTYEYVSKSFQLDVSEKNGNFKH
LREFVFKNVDGFLHVYSAYEPIDLARGLPSGFSVLKPILKLPLGINITSFRVVMTMESPTTS
NWLAESAAYFVGYLKPTTFMLKFNENGTITDAVDCSQDPLSELKCTLKSFNVEKGIYQTSNF
RVSPTQEVVRFPNITNLCPFDKVFNATRFPSVYAWERTKISDCVADYTVLYNSTSFSTFKCY
GVSPSKLIDLCFTSVYADTFLIRSSEVRQVAPGQTGVIADYNYKLPDDFTGCVIAWNTAKQD
AGNYYYRSHRKTKLKPFERDLSNSDENGVRTLSTYDFNPNVPIEYQATRVVVLSFELLNAPA
TVCGPKLSTQLVKNQCVNFNFNGLKGTGVLTDSSKRFQSFQQFGRDASDFTDSVRDPQTLEI
LDISPCSFGGVSVITPGTNASSEVAVLYQDVNCTDVPTAIHADQLTPAWRVYSTGVNVFQTQ
AGCLIGAEHVNASYECDIPIGAGICASYHTASTLRSTGQKSIVAYTMSLGAENSIAYANNSI
AIPTNFSISVTTEVMPVSMAKTSVDCTMYICGDSQECSNLLLQYGSFCTQLNRALTGIAIEQ
DKNTQEVFAQVKQMYKTPAIKDFGGFNFSQILPDPSKPTKRSFIEDLLFNKVTLADAGFMKQ
YGECLGDISARDLICAQKFNGLTVLPPLLTDEMIAAYTAALVSGTATAGWTFGAGAALQIPF
AMQMAYRFNGIGVTQNVLYENQKQIANQFNKAISQIQESLTTTSTALGKLQDVVNQNAQALN
TLVKQLSSNFGAISSVLNDILSRLDKVEAEVQIDRLITGRLQSLQTYVTQQLIRAAEIRASA
NLAATKMSECVLGQSKRVDFCGKGYHLMSFPQAAPHGVVFLHVTYVPSQERNFTTAPAICHE
GKAYFPREGVFVSNGTSWFITQRNFYSPQIITTDNTFVAGNCDVVIGIINNTVYDPLQPELD
SFKEELDKYFKNHTSPDVDLGDISGINASVVNIQKEIDRLNEVAKNLNESLIDLQELGKYEQ
GSGYIPEAPRDGQAYVRKDGEWVLLSTFLGTSLEVLFQGPGHHHHHHHHSAWSHPQFEKGGG
SGGGGSGGSAWSHPQFEK
A5 without C-terminal additions
(SEQ ID NO: 13)
MFVFLVLLPLVSSTAQEGTCGTISNKTPPNMNQFSSSRRGVYYPDDIFRSDVLHLTQDYFLP
FNSNVTRYLSLNADSNRIVRFDNPILPFGDGIYFAATEKSNVIRGWIFGSTLDNTSQSAIIV
NNSTHIIIKVCNFQLCDDPMFTVSRGQHYKTWVYTNARNCTYEYVSKSFQLDVSEKNGNFKH
LREFVFKNVDGFLHVYSAYEPIDLARGLPSGFSVLKPILKLPLGINITSFRVVMTMFSPTTS
NWLAESAAYFVGYLKPTTFMLKFNENGTITDAVDCSQDPLSELKCTLKSFNVEKGIYQTSNF
RVSPTQEVVRFPNITNLCPFDKVFNATRFPSVYAWERTKISDCVADYTVLYNSTSFSTFKCY
GVSPSKLIDLCFTSVYADTFLIRSSEVRQVAPGQTGVIADYNYKLPDDFTGCVIAWNTAKQD
AGNYYYRSHRKTKLKPFERDLSNSDENGVRTLSTYDFNPNVPIEYQATRVVVLSFELLNAPA
TVCGPKLSTQLVKNQCVNFNFNGLKGTGVLTDSSKRFQSFQQFGRDASDFTDSVRDPQTLEI
LDISPCSFGGVSVITPGTNASSEVAVLYQDVNCTDVPTAIHADQLTPAWRVYSTGVNVFQTQ
AGCLIGAEHVNASYECDIPIGAGICASYHTASTLRSTGQKSIVAYTMSLGAENSIAYANNSI
AIPTNFSISVTTEVMPVSMAKTSVDCTMYICGDSQECSNLLLQYGSFCTQLNRALTGIAIEQ
DKNTQEVFAQVKQMYKTPAIKDFGGFNFSQILPDPSKPTKRSFIEDLLFNKVTLADAGEMKQ
YGECLGDISARDLICAQKFNGLTVLPPLLTDEMIAAYTAALVSGTATAGWTFGAGAALQIPF
AMQMAYRFNGIGVTQNVLYENQKQIANQFNKAISQIQESLTTTSTALGKLQDVVNQNAQALN
TLVKQLSSNFGAISSVLNDILSRLDKVEAEVQIDRLITGRLQSLQTYVTQQLIRAAEIRASA
NLAATKMSECVLGQSKRVDFCGKGYHLMSFPQAAPHGVVFLHVTYVPSQERNFTTAPAICHE
GKAYFPREGVFVSNGTSWFITQRNFYSPQIITTDNTFVAGNCDVVIGIINNTVYDPLQPELD
SFKEELDKYFKNHTSPDVDLGDISGINASVVNIQKEIDRLNEVAKNLNESLIDLQELGKYEQ
A5 without N-terminal signal peptide and C-terminal additions
(SEQ ID NO: 14)
TAQEGTCGTISNKTPPNMNQFSSSRRGVYYPDDIFRSDVLHLTQDYFLPFNSNVTRYLSLNA
DSNRIVRFDNPILPFGDGIYFAATEKSNVIRGWIFGSTLDNTSQSAIIVNNSTHIIIKVCNF
QLCDDPMFTVSRGQHYKTWVYTNARNCTYEYVSKSFQLDVSEKNGNFKHLREFVFKNVDGFL
HVYSAYEPIDLARGLPSGFSVLKPILKLPLGINITSFRVVMTMESPTTSNWLAESAAYFVGY
LKPTTFMLKFNENGTITDAVDCSQDPLSELKCTLKSFNVEKGIYQTSNFRVSPTQEVVRFPN
ITNLCPFDKVFNATRFPSVYAWERTKISDCVADYTVLYNSTSFSTFKCYGVSPSKLIDLCFT
SVYADTFLIRSSEVRQVAPGQTGVIADYNYKLPDDFTGCVIAWNTAKQDAGNYYYRSHRKTK
LKPFERDLSNSDENGVRTLSTYDFNPNVPIEYQATRVVVLSFELLNAPATVCGPKLSTQLVK
NQCVNFNFNGLKGTGVLTDSSKRFQSFQQFGRDASDFTDSVRDPQTLEILDISPCSFGGVSV
ITPGTNASSEVAVLYQDVNCTDVPTAIHADQLTPAWRVYSTGVNVFQTQAGCLIGAEHVNAS
YECDIPIGAGICASYHTASTLRSTGQKSIVAYTMSLGAENSIAYANNSIAIPTNFSISVTTE
VMPVSMAKTSVDCTMYICGDSQECSNLLLQYGSFCTQLNRALTGIAIEQDKNTQEVFAQVKQ
MYKTPAIKDFGGFNFSQILPDPSKPTKRSFIEDLLFNKVTLADAGEMKQYGECLGDISARDL
ICAQKFNGLTVLPPLLTDEMIAAYTAALVSGTATAGWTFGAGAALQIPFAMQMAYRFNGIGV
TQNVLYENQKQIANQFNKAISQIQESLTTTSTALGKLQDVVNQNAQALNTLVKQLSSNFGAI
SSVLNDILSRLDKVEAEVQIDRLITGRLQSLQTYVTQQLIRAAEIRASANLAATKMSECVLG
QSKRVDFCGKGYHLMSFPQAAPHGVVFLHVTYVPSQERNFTTAPAICHEGKAYFPREGVFVS
NGTSWFITQRNFYSPQIITTDNTFVAGNCDVVIGIINNTVYDPLQPELDSFKEELDKYFKNH
TSPDVDLGDISGINASVVNIQKEIDRLNEVAKNLNESLIDLQELGKYEQ
A5-part 1 without N-terminal signal peptide
(SEQ ID NO: 15)
TAQEGTCGTISNKTPPNMNQFSSSRRGVYYPDDIFRSDVLHLTQDYFLPFNSNVTRYLSLNA
DSNRIVRFDNPILPFGDGIYFAATEKSNVIRGWIFGSTLDNTSQSAIIVNNSTHIIIKVCNF
QLCDDPMFTVSRGQHYKTWVYTNARNCTYEYVSKSFQLDVSEKNGNFKHLREFVFKNVDGFL
HVYSAYEPIDLARGLPSGFSVLKPILKLPLGINITSFRVVMTMFSPTTSNWLAESAAYFVGY
LKPTTFMLKFNENGTITDAVDCSQDPLSELKCTLKSFNVEKGIYQTSNF
A5-part 1
(SEQ ID NO: 16)
MFVFLVLLPLVSSTAQEGTCGTISNKTPPNMNQFSSSRRGVYYPDDIFRSDVLHLTQDYFLP
FNSNVTRYLSLNADSNRIVRFDNPILPFGDGIYFAATEKSNVIRGWIFGSTLDNTSQSAIIV
NNSTHIIIKVCNFQLCDDPMFTVSRGQHYKTWVYTNARNCTYEYVSKSFQLDVSEKNGNFKH
LREFVFKNVDGFLHVYSAYEPIDLARGLPSGFSVLKPILKLPLGINITSFRVVMTMFSPTTS
NWLAESAAYFVGYLKPTTFMLKFNENGTITDAVDCSQDPLSELKCTLKSFNVEKGIYQTSNF
A5 RBD
(SEQ ID NO: 17)
RVSPTQEVVRFPNITNLCPFDKVFNATRFPSVYAWERTKISDCVADYTVLYNSTSFSTFKCY
GVSPSKLIDLCFTSVYADTFLIRSSEVRQVAPGQTGVIADYNYKLPDDFTGCVIAWNTAKQD
AGNYYYRSHRKTKLKPFERDLSNSDENGVRTLSTYDFNPNVPIEYQATRVVVLSFELLNAPA
TVCGPKLSTQLVKNQCVNF
A5-part 2
(SEQ ID NO: 18)
NFNGLKGTGVLTDSSKRFQSFQQFGRDASDFTDSVRDPQTLEILDISPCSFGGVSVITPGTN
ASSEVAVLYQDVNCTDVPTAIHADQLTPAWRVYSTGVNVFQTQAGCLIGAEHVNASYECDIP
IGAGICASYHTASTLRSTGQKSIVAYTMSLGAENSIAYANNSIAIPTNFSISVTTEVMPVSM
AKTSVDCTMYICGDSQECSNLLLQYGSFCTQLNRALTGIAIEQDKNTQEVFAQVKQMYKTPA
IKDFGGFNFSQILPDPSKPTKRSFIEDLLENKVTLADAGFMKQYGECLGDISARDLICAQKF
NGLTVLPPLLTDEMIAAYTAALVSGTATAGWTFGAGAALQIPFAMQMAYRFNGIGVTQNVLY
ENQKQIANQFNKAISQIQESLTTTSTALGKLQDVVNQNAQALNTLVKQLSSNFGAISSVLND
ILSRLDKVEAEVQIDRLITGRLQSLQTYVTQQLIRAAEIRASANLAATKMSECVLGQSKRVD
FCGKGYHLMSFPQAAPHGVVFLHVTYVPSQERNFTTAPAICHEGKAYFPREGVFVSNGTSWF
ITQRNFYSPQIITTDNTFVAGNCDVVIGIINNTVYDPLQPELDSFKEELDKYFKNHTSPDVD
LGDISGINASVVNIQKEIDRLNEVAKNLNESLIDLQELGKYEQ
A5 with wt RBD
(SEQ ID NO: 19)
MFVFLVLLPLVSSTAQEGTCGTISNKTPPNMNQFSSSRRGVYYPDDIFRSDVLHLTQDYFLP
FNSNVTRYLSLNADSNRIVRFDNPILPFGDGIYFAATEKSNVIRGWIFGSTLDNTSQSAIIV
NNSTHIIIKVCNFQLCDDPMFTVSRGQHYKTWVYTNARNCTYEYVSKSFQLDVSEKNGNFKH
LREFVFKNVDGFLHVYSAYEPIDLARGLPSGFSVLKPILKLPLGINITSFRVVMTMFSPTTS
NWLAESAAYFVGYLKPTTFMLKFNENGTITDAVDCSQDPLSELKCTLKSFNVEKGIYQTSNF
RVQPTESIVRFPNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLD
SKVGGNYNYLYRLFRKSNLKPFERDISTEIYQAGSTPCNGVEGFNCYFPLQSYGFQPTNGVG
YQPYRVVVLSFELLHAPATVCGPKKSTNLVKNKCVNFNFNGLKGTGVLTDSSKRFQSFQQFG
RDASDFTDSVRDPQTLEILDISPCSFGGVSVITPGTNASSEVAVLYQDVNCTDVPTAIHADQ
LTPAWRVYSTGVNVFQTQAGCLIGAEHVNASYECDIPIGAGICASYHTASTLRSTGQKSIVA
YTMSLGAENSIAYANNSIAIPTNFSISVTTEVMPVSMAKTSVDCTMYICGDSQECSNLLLQY
GSFCTQLNRALTGIAIEQDKNTQEVFAQVKQMYKTPAIKDFGGFNFSQILPDPSKPTKRSFI
EDLLFNKVTLADAGEMKQYGECLGDISARDLICAQKFNGLTVLPPLLTDEMIAAYTAALVSG
TATAGWTFGAGAALQIPFAMQMAYRFNGIGVTQNVLYENQKQIANQFNKAISQIQESLTTTS
TALGKLQDVVNQNAQALNTLVKQLSSNFGAISSVLNDILSRLDKVEAEVQIDRLITGRLQSL
QTYVTQQLIRAAEIRASANLAATKMSECVLGQSKRVDFCGKGYHLMSFPQAAPHGVVFLHVT
YVPSQERNFTTAPAICHEGKAYFPREGVFVSNGTSWFITQRNFYSPQIITTDNTFVAGNCDV
VIGIINNTVYDPLQPELDSFKEELDKYFKNHTSPDVDLGDISGINASVVNIQKEIDRLNEVA
KNLNESLIDLQELGKYEQGSGYIPEAPRDGQAYVRKDGEWVLLSTFLGTSLEVLFQGPGHHH
HHHHHSAWSHPQFEKGGGSGGGGSGGSAWSHPQFEK
A5 with wt RBD without C-terminal additions
(SEQ ID NO: 20)
MFVFLVLLPLVSSTAQEGTCGTISNKTPPNMNQFSSSRRGVYYPDDIFRSDVLHLTQDYFLP
FNSNVTRYLSLNADSNRIVRFDNPILPFGDGIYFAATEKSNVIRGWIFGSTLDNTSQSAIIV
NNSTHIIIKVCNFQLCDDPMFTVSRGQHYKTWVYTNARNCTYEYVSKSFQLDVSEKNGNFKH
LREFVFKNVDGFLHVYSAYEPIDLARGLPSGFSVLKPILKLPLGINITSFRVVMTMFSPTTS
NWLAESAAYFVGYLKPTTFMLKFNENGTITDAVDCSQDPLSELKCTLKSFNVEKGIYQTSNF
RVQPTESIVRFPNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLD
SKVGGNYNYLYRLFRKSNLKPFERDISTEIYQAGSTPCNGVEGFNCYFPLQSYGFQPTNGVG
YQPYRVVVLSFELLHAPATVCGPKKSTNLVKNKCVNFNFNGLKGTGVLTDSSKRFQSFQQFG
RDASDFTDSVRDPQTLEILDISPCSFGGVSVITPGTNASSEVAVLYQDVNCTDVPTAIHADQ
LTPAWRVYSTGVNVFQTQAGCLIGAEHVNASYECDIPIGAGICASYHTASTLRSTGQKSIVA
YTMSLGAENSIAYANNSIAIPTNFSISVTTEVMPVSMAKTSVDCTMYICGDSQECSNLLLQY
GSFCTQLNRALTGIAIEQDKNTQEVFAQVKQMYKTPAIKDFGGFNFSQILPDPSKPTKRSFI
EDLLFNKVTLADAGEMKQYGECLGDISARDLICAQKENGLTVLPPLLTDEMIAAYTAALVSG
TATAGWTFGAGAALQIPFAMQMAYRFNGIGVTQNVLYENQKQIANQFNKAISQIQESLTTTS
TALGKLQDVVNQNAQALNTLVKQLSSNFGAISSVLNDILSRLDKVEAEVQIDRLITGRLQSL
QTYVTQQLIRAAEIRASANLAATKMSECVLGQSKRVDFCGKGYHLMSFPQAAPHGVVFLHVT
YVPSQERNFTTAPAICHEGKAYFPREGVFVSNGTSWFITQRNFYSPQIITTDNTFVAGNCDV
VIGIINNTVYDPLQPELDSFKEELDKYFKNHTSPDVDLGDISGINASVVNIQKEIDRLNEVA
KNLNESLIDLQELGKYEQ
A5 with wt RBD without N-terminal signal peptide and C-terminal
additions
(SEQ ID NO: 21)
TAQEGTCGTISNKTPPNMNQFSSSRRGVYYPDDIFRSDVLHLTQDYFLPFNSNVTRYLSLNA
DSNRIVRFDNPILPFGDGIYFAATEKSNVIRGWIFGSTLDNTSQSAIIVNNSTHIIIKVCNF
QLCDDPMFTVSRGQHYKTWVYTNARNCTYEYVSKSFQLDVSEKNGNFKHLREFVFKNVDGFL
HVYSAYEPIDLARGLPSGFSVLKPILKLPLGINITSFRVVMTMFSPTTSNWLAESAAYFVGY
LKPTTFMLKFNENGTITDAVDCSQDPLSELKCTLKSFNVEKGIYQTSNERVQPTESIVRFPN
ITNLCPFGEVFNATRFASVYAWNRKRISNCVADYSVLYNSASFSTFKCYGVSPTKLNDLCFT
NVYADSFVIRGDEVRQIAPGQTGKIADYNYKLPDDFTGCVIAWNSNNLDSKVGGNYNYLYRL
FRKSNLKPFERDISTEIYQAGSTPCNGVEGENCYFPLQSYGFQPTNGVGYQPYRVVVLSFEL
LHAPATVCGPKKSTNLVKNKCVNFNFNGLKGTGVLTDSSKRFQSFQQFGRDASDETDSVRDP
QTLEILDISPCSFGGVSVITPGTNASSEVAVLYQDVNCTDVPTAIHADQLTPAWRVYSTGVN
VFQTQAGCLIGAEHVNASYECDIPIGAGICASYHTASTLRSTGQKSIVAYTMSLGAENSIAY
ANNSIAIPTNFSISVTTEVMPVSMAKTSVDCTMYICGDSQECSNLLLQYGSFCTQLNRALTG
IAIEQDKNTQEVFAQVKQMYKTPAIKDFGGFNFSQILPDPSKPTKRSFIEDLLFNKVTLADA
GFMKQYGECLGDISARDLICAQKFNGLTVLPPLLTDEMIAAYTAALVSGTATAGWTFGAGAA
LQIPFAMQMAYRFNGIGVTQNVLYENQKQIANQFNKAISQIQESLTTTSTALGKLQDVVNQN
AQALNTLVKQLSSNFGAISSVLNDILSRLDKVEAEVQIDRLITGRLQSLQTYVTQQLIRAAE
IRASANLAATKMSECVLGQSKRVDFCGKGYHLMSFPQAAPHGVVFLHVTYVPSQERNFTTAP
AICHEGKAYFPREGVFVSNGTSWFITQRNFYSPQIITTDNTFVAGNCDVVIGIINNTVYDPL
QPELDSFKEELDKYFKNHTSPDVDLGDISGINASVVNIQKEIDRLNEVAKNLNESLIDLQEL
GKYEQ
A6
(SEQ ID NO: 22)
MFVFLVLLPLVSSTAQEGTCGTLSNKSPPNMTQFSSSRRGVYYPDDIFRSDVLHLTQDYFLP
FNSNVTRYLSLNSDSDRIVRFDNPIIPFGDGVYFAATEKSNVIRGWIFGSTLDNTSQSAIIM
NNSTHIVIRVCNFQLCDDPMFAVSRPTGQHYKTWIYTNARNCTYEYVSKSFQLDVSEKPGNF
KHLREFVFKNVDGFLHVYSGYEPIDVARGLPSGFSVLKPIFKLPLGINITNFRVIMTMFSPT
TSNWGAEAAAYFVGYLKPTTFMLKFDENGTITDAVDCSQDPLSELKCTVKSFNVEKGIYQTS
NFRVSPTKEVVRFPNITNLCPFGEVFNATTFPSVYAWERTRISDCVADYSVLYNSTSFSTFK
CYGVSPTKLNDLCFSSVYADSFVVKGDDVRQIAPGQTGVIADYNYKLPDDFTGCVIAWNTAN
LDATSTGNYNYYYRSLRHGKLKPFERDISNVPFSPEGKPCTPPAFNCYRPLNTYGFNPTVGI
GYQPYRVVVLSFELLNAPATVCGPKLSTELVKNQCVNFNFNGLTGTGVLTDSSKRFQPFQQF
GRDVSDFTDSVRDPKTLEILDISPCSFGGVSVITPGTNTSSEVAVLYQDVNCTDVPTAIHAD
QLTPAWRVYSTGVNVFQTQAGCLIGAEHVNASYECDIPIGAGICASYHTASTLRSTGQKSIV
AYTMSLGAENSIAYSNNTIAIPTNFSISVTTEVMPVSMAKTSVDCTMYICGDSTECSNLLLQ
YGSFCTQLNRALSGIAVEQDKNTREVFAQVKQMYKTPAIKDFGGFNFSQILPDPSKPTKRSF
IEDLLFNKVTLADAGFMKQYGECLGDISARDLICAQKFNGLTVLPPLLTDEMIAAYTAALVS
GTATAGWTFGAGAALQIPFAMQMAYRFNGIGVTQNVLYENQKQIANQFNKAISQIQESLTTT
STALGKLQDVVNQNAQALNTLVKQLSSNFGAISSVLNDILSRLDKVEAEVQIDRLITGRLQS
LQTYVTQQLIRAAEIRASANLAATKMSECVLGQSKRVDFCGKGYHLMSFPQAAPHGVVFLHV
TYVPSQERNFTTAPAICHEGKAYFPREGVFVSNGTSWFITQRNFYSPQIITTDNTFVSGNCD
VVIGIINNTVYDPLQPELDSFKEELDKYFKNHTSPDVDLGDISGINASVVNIQKEIDRLNEV
AKNLNESLIDLQELGKYEQGSGYIPEAPRDGQAYVRKDGEWVLLSTFLGTSLEVLFQGPGHH
HHHHHHSAWSHPQFEKGGGSGGGGSGGSAWSHPQFEK
A6 without C-terminal additions
(SEQ ID NO: 23)
MFVFLVLLPLVSSTAQEGTCGTLSNKSPPNMTQFSSSRRGVYYPDDIFRSDVLHLTQDYFLP
FNSNVTRYLSLNSDSDRIVRFDNPIIPFGDGVYFAATEKSNVIRGWIFGSTLDNTSQSAIIM
NNSTHIVIRVCNFQLCDDPMFAVSRPTGQHYKTWIYTNARNCTYEYVSKSFQLDVSEKPGNF
KHLREFVFKNVDGFLHVYSGYEPIDVARGLPSGFSVLKPIFKLPLGINITNFRVIMTMFSPT
TSNWGAEAAAYFVGYLKPTTFMLKFDENGTITDAVDCSQDPLSELKCTVKSFNVEKGIYQTS
NFRVSPTKEVVRFPNITNLCPFGEVFNATTFPSVYAWERTRISDCVADYSVLYNSTSFSTFK
CYGVSPTKLNDLCFSSVYADSFVVKGDDVRQIAPGQTGVIADYNYKLPDDFTGCVIAWNTAN
LDATSTGNYNYYYRSLRHGKLKPFERDISNVPFSPEGKPCTPPAFNCYRPLNTYGFNPTVGI
GYQPYRVVVLSFELLNAPATVCGPKLSTELVKNQCVNFNFNGLTGTGVLTDSSKRFQPFQQF
GRDVSDFTDSVRDPKTLEILDISPCSFGGVSVITPGTNTSSEVAVLYQDVNCTDVPTAIHAD
QLTPAWRVYSTGVNVFQTQAGCLIGAEHVNASYECDIPIGAGICASYHTASTLRSTGQKSIV
AYTMSLGAENSIAYSNNTIAIPTNFSISVTTEVMPVSMAKTSVDCTMYICGDSTECSNLLLQ
YGSFCTQLNRALSGIAVEQDKNTREVFAQVKQMYKTPAIKDFGGFNFSQILPDPSKPTKRSF
IEDLLFNKVTLADAGFMKQYGECLGDISARDLICAQKFNGLTVLPPLLTDEMIAAYTAALVS
GTATAGWTFGAGAALQIPFAMQMAYRFNGIGVTQNVLYENQKQIANQFNKAISQIQESLTTT
STALGKLQDVVNQNAQALNTLVKQLSSNFGAISSVLNDILSRLDKVEAEVQIDRLITGRLQS
LQTYVTQQLIRAAEIRASANLAATKMSECVLGQSKRVDFCGKGYHLMSFPQAAPHGVVFLHV
TYVPSQERNFTTAPAICHEGKAYFPREGVFVSNGTSWFITQRNFYSPQIITTDNTFVSGNCD
VVIGIINNTVYDPLQPELDSFKEELDKYFKNHTSPDVDLGDISGINASVVNIQKEIDRLNEV
AKNLNESLIDLQELGKYEQ
A6 without N-terminal signal peptide and C-terminal additions
(SEQ ID NO: 24)
TAQEGTCGTLSNKSPPNMTQFSSSRRGVYYPDDIFRSDVLHLTQDYFLPFNSNVTRYLSLNS
DSDRIVRFDNPIIPFGDGVYFAATEKSNVIRGWIFGSTLDNTSQSAIIMNNSTHIVIRVCNF
QLCDDPMFAVSRPTGQHYKTWIYTNARNCTYEYVSKSFQLDVSEKPGNFKHLREFVFKNVDG
FLHVYSGYEPIDVARGLPSGFSVLKPIFKLPLGINITNFRVIMTMFSPTTSNWGAEAAAYFV
GYLKPTTFMLKFDENGTITDAVDCSQDPLSELKCTVKSFNVEKGIYQTSNFRVSPTKEVVRF
PNITNLCPFGEVENATTFPSVYAWERTRISDCVADYSVLYNSTSFSTFKCYGVSPTKLNDLC
FSSVYADSFVVKGDDVRQIAPGQTGVIADYNYKLPDDFTGCVIAWNTANLDATSTGNYNYYY
RSLRHGKLKPFERDISNVPFSPEGKPCTPPAFNCYRPLNTYGFNPTVGIGYQPYRVVVLSFE
LLNAPATVCGPKLSTELVKNQCVNFNFNGLTGTGVLTDSSKRFQPFQQFGRDVSDFTDSVRD
PKTLEILDISPCSFGGVSVITPGTNTSSEVAVLYQDVNCTDVPTAIHADQLTPAWRVYSTGV
NVFQTQAGCLIGAEHVNASYECDIPIGAGICASYHTASTLRSTGQKSIVAYTMSLGAENSIA
YSNNTIAIPTNFSISVTTEVMPVSMAKTSVDCTMYICGDSTECSNLLLQYGSFCTQLNRALS
GIAVEQDKNTREVFAQVKQMYKTPAIKDFGGFNFSQILPDPSKPTKRSFIEDLLFNKVTLAD
AGFMKQYGECLGDISARDLICAQKFNGLTVLPPLLTDEMIAAYTAALVSGTATAGWTFGAGA
ALQIPFAMQMAYRENGIGVTQNVLYENQKQIANQFNKAISQIQESLTTTSTALGKLQDVVNQ
NAQALNTLVKQLSSNFGAISSVLNDILSRLDKVEAEVQIDRLITGRLQSLQTYVTQQLIRAA
EIRASANLAATKMSECVLGQSKRVDFCGKGYHLMSFPQAAPHGVVFLHVTYVPSQERNFTTA
PAICHEGKAYFPREGVFVSNGTSWFITQRNFYSPQIITTDNTFVSGNCDVVIGIINNTVYDP
LQPELDSFKEELDKYFKNHTSPDVDLGDISGINASVVNIQKEIDRLNEVAKNLNESLIDLQE
LGKYEQ
A6-part 1 without N-terminal signal peptide
(SEQ ID NO: 25)
TAQEGTCGTLSNKSPPNMTQFSSSRRGVYYPDDIFRSDVLHLTQDYFLPFNSNVTRYLSLNS
DSDRIVRFDNPIIPFGDGVYFAATEKSNVIRGWIFGSTLDNTSQSAIIMNNSTHIVIRVCNF
QLCDDPMFAVSRPTGQHYKTWIYTNARNCTYEYVSKSFQLDVSEKPGNFKHLREFVFKNVDG
FLHVYSGYEPIDVARGLPSGFSVLKPIFKLPLGINITNFRVIMTMFSPTTSNWGAEAAAYFV
GYLKPTTFMLKFDENGTITDAVDCSQDPLSELKCTVKSFNVEKGIYQTSNF
A6-part 1
(SEQ ID NO: 26)
MFVFLVLLPLVSSTAQEGTCGTLSNKSPPNMTQFSSSRRGVYYPDDIFRSDVLHLTQDYFLP
FNSNVTRYLSLNSDSDRIVRFDNPIIPFGDGVYFAATEKSNVIRGWIFGSTLDNTSQSAIIM
NNSTHIVIRVCNFQLCDDPMFAVSRPTGQHYKTWIYTNARNCTYEYVSKSFQLDVSEKPGNF
KHLREFVFKNVDGFLHVYSGYEPIDVARGLPSGFSVLKPIFKLPLGINITNFRVIMTMFSPT
TSNWGAEAAAYFVGYLKPTTEMLKFDENGTITDAVDCSQDPLSELKCTVKSFNVEKGIYQTS
NF
A6 RBD
(SEQ ID NO: 27)
RVSPTKEVVRFPNITNLCPFGEVFNATTFPSVYAWERTRISDCVADYSVLYNSTSFSTFKCY
GVSPTKLNDLCFSSVYADSFVVKGDDVRQIAPGQTGVIADYNYKLPDDFTGCVIAWNTANLD
ATSTGNYNYYYRSLRHGKLKPFERDISNVPFSPEGKPCTPPAFNCYRPLNTYGFNPTVGIGY
QPYRVVVLSFELLNAPATVCGPKLSTELVKNQCVNF
A6-part 2
(SEQ ID NO: 28)
NFNGLTGTGVLTDSSKRFQPFQQFGRDVSDFTDSVRDPKTLEILDISPCSFGGVSVITPGTN
TSSEVAVLYQDVNCTDVPTAIHADQLTPAWRVYSTGVNVFQTQAGCLIGAEHVNASYECDIP
IGAGICASYHTASTLRSTGQKSIVAYTMSLGAENSIAYSNNTIAIPTNFSISVTTEVMPVSM
AKTSVDCTMYICGDSTECSNLLLQYGSFCTQLNRALSGIAVEQDKNTREVFAQVKQMYKTPA
IKDFGGFNFSQILPDPSKPTKRSFIEDLLFNKVTLADAGFMKQYGECLGDISARDLICAQKF
NGLTVLPPLLTDEMIAAYTAALVSGTATAGWTFGAGAALQIPFAMQMAYRFNGIGVTQNVLY
ENQKQIANQFNKAISQIQESLTTTSTALGKLQDVVNQNAQALNTLVKQLSSNFGAISSVLND
ILSRLDKVEAEVQIDRLITGRLQSLQTYVTQQLIRAAEIRASANLAATKMSECVLGQSKRVD
FCGKGYHLMSFPQAAPHGVVFLHVTYVPSQERNFTTAPAICHEGKAYFPREGVFVSNGTSWF
ITQRNFYSPQIITTDNTFVSGNCDVVIGIINNTVYDPLQPELDSFKEELDKYFKNHTSPDVD
LGDISGINASVVNIQKEIDRLNEVAKNLNESLIDLQELGKYEQ
A6 with wt RBD
(SEQ ID NO: 29)
MFVFLVLLPLVSSTAQEGTCGTLSNKSPPNMTQFSSSRRGVYYPDDIFRSDVLHLTQDYFLP
FNSNVTRYLSLNSDSDRIVRFDNPIIPFGDGVYFAATEKSNVIRGWIFGSTLDNTSQSAIIM
NNSTHIVIRVCNFQLCDDPMFAVSRPTGQHYKTWIYTNARNCTYEYVSKSFQLDVSEKPGNF
KHLREFVFKNVDGFLHVYSGYEPIDVARGLPSGFSVLKPIFKLPLGINITNFRVIMTMFSPT
TSNWGAEAAAYFVGYLKPTTEMLKFDENGTITDAVDCSQDPLSELKCTVKSFNVEKGIYQTS
NFRVQPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGFNCYFPLQSYGFQPTNG
VGYQPYRVVVLSFELLHAPATVCGPKKSTNLVKNKCVNFNFNGLTGTGVLTDSSKRFQPFQQ
FGRDVSDFTDSVRDPKTLEILDISPCSFGGVSVITPGTNTSSEVAVLYQDVNCTDVPTAIHA
DQLTPAWRVYSTGVNVFQTQAGCLIGAEHVNASYECDIPIGAGICASYHTASTLRSTGQKSI
VAYTMSLGAENSIAYSNNTIAIPTNFSISVTTEVMPVSMAKTSVDCTMYICGDSTECSNLLL
QYGSFCTQLNRALSGIAVEQDKNTREVFAQVKQMYKTPAIKDFGGFNFSQILPDPSKPTKRS
FIEDLLFNKVTLADAGFMKQYGECLGDISARDLICAQKFNGLTVLPPLLTDEMIAAYTAALV
SGTATAGWTFGAGAALQIPFAMQMAYRFNGIGVTQNVLYENQKQIANQFNKAISQIQESLTT
TSTALGKLQDVVNQNAQALNTLVKQLSSNFGAISSVLNDILSRLDKVEAEVQIDRLITGRLQ
SLQTYVTQQLIRAAEIRASANLAATKMSECVLGQSKRVDFCGKGYHLMSFPQAAPHGVVFLH
VTYVPSQERNFTTAPAICHEGKAYFPREGVFVSNGTSWFITQRNFYSPQIITTDNTFVSGNC
DVVIGIINNTVYDPLQPELDSFKEELDKYFKNHTSPDVDLGDISGINASVVNIQKEIDRLNE
VAKNLNESLIDLQELGKYEQGSGYIPEAPRDGQAYVRKDGEWVLLSTFLGTSLEVLFQGPGH
HHHHHHHSAWSHPQFEKGGGSGGGGSGGSAWSHPQFEK
A6 with wt RBD without C-terminal additions
(SEQ ID NO: 30)
MFVFLVLLPLVSSTAQEGTCGTLSNKSPPNMTQFSSSRRGVYYPDDIFRSDVLHLTQDYFLP
FNSNVTRYLSLNSDSDRIVRFDNPIIPFGDGVYFAATEKSNVIRGWIFGSTLDNTSQSAIIM
NNSTHIVIRVCNFQLCDDPMFAVSRPTGQHYKTWIYTNARNCTYEYVSKSFQLDVSEKPGNF
KHLREFVFKNVDGFLHVYSGYEPIDVARGLPSGFSVLKPIFKLPLGINITNFRVIMTMFSPT
TSNWGAEAAAYFVGYLKPTTFMLKFDENGTITDAVDCSQDPLSELKCTVKSFNVEKGIYQTS
NFRVQPTESIVRFPNITNLCPFGEVENATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGENCYFPLQSYGFQPTNG
VGYQPYRVVVLSFELLHAPATVCGPKKSTNLVKNKCVNFNFNGLTGTGVLTDSSKRFQPFQQ
FGRDVSDFTDSVRDPKTLEILDISPCSFGGVSVITPGTNTSSEVAVLYQDVNCTDVPTAIHA
DQLTPAWRVYSTGVNVFQTQAGCLIGAEHVNASYECDIPIGAGICASYHTASTLRSTGQKSI
VAYTMSLGAENSIAYSNNTIAIPTNFSISVTTEVMPVSMAKTSVDCTMYICGDSTECSNLLL
QYGSFCTQLNRALSGIAVEQDKNTREVFAQVKQMYKTPAIKDFGGFNFSQILPDPSKPTKRS
FIEDLLFNKVTLADAGEMKQYGECLGDISARDLICAQKENGLTVLPPLLTDEMIAAYTAALV
SGTATAGWTFGAGAALQIPFAMQMAYRFNGIGVTQNVLYENQKQIANQFNKAISQIQESLTT
TSTALGKLQDVVNQNAQALNTLVKQLSSNFGAISSVLNDILSRLDKVEAEVQIDRLITGRLQ
SLQTYVTQQLIRAAEIRASANLAATKMSECVLGQSKRVDFCGKGYHLMSFPQAAPHGVVFLH
VTYVPSQERNFTTAPAICHEGKAYFPREGVFVSNGTSWFITQRNFYSPQIITTDNTFVSGNC
DVVIGIINNTVYDPLQPELDSFKEELDKYFKNHTSPDVDLGDISGINASVVNIQKEIDRLNE
VAKNLNESLIDLQELGKYEQ
A6 with wt RBD without N-terminal signal peptide and C-terminal
additions
(SEQ ID NO: 31)
TAQEGTCGTLSNKSPPNMTQFSSSRRGVYYPDDIFRSDVLHLTQDYFLPFNSNVTRYLSLNS
DSDRIVRFDNPIIPFGDGVYFAATEKSNVIRGWIFGSTLDNTSQSAIIMNNSTHIVIRVCNF
QLCDDPMFAVSRPTGQHYKTWIYTNARNCTYEYVSKSFQLDVSEKPGNFKHLREFVFKNVDG
FLHVYSGYEPIDVARGLPSGFSVLKPIFKLPLGINITNFRVIMTMFSPTTSNWGAEAAAYFV
GYLKPTTFMLKFDENGTITDAVDCSQDPLSELKCTVKSFNVEKGIYQTSNERVQPTESIVRF
PNITNLCPFGEVFNATRFASVYAWNRKRISNCVADYSVLYNSASFSTFKCYGVSPTKLNDLC
FTNVYADSFVIRGDEVRQIAPGQTGKIADYNYKLPDDFTGCVIAWNSNNLDSKVGGNYNYLY
RLFRKSNLKPFERDISTEIYQAGSTPCNGVEGENCYFPLQSYGFQPTNGVGYQPYRVVVLSF
ELLHAPATVCGPKKSTNLVKNKCVNFNFNGLTGTGVLTDSSKRFQPFQQFGRDVSDFTDSVR
DPKTLEILDISPCSFGGVSVITPGTNTSSEVAVLYQDVNCTDVPTAIHADQLTPAWRVYSTG
VNVFQTQAGCLIGAEHVNASYECDIPIGAGICASYHTASTLRSTGQKSIVAYTMSLGAENSI
AYSNNTIAIPTNFSISVTTEVMPVSMAKTSVDCTMYICGDSTECSNLLLQYGSFCTQLNRAL
SGIAVEQDKNTREVFAQVKQMYKTPAIKDFGGFNFSQILPDPSKPTKRSFIEDLLFNKVTLA
DAGFMKQYGECLGDISARDLICAQKFNGLTVLPPLLTDEMIAAYTAALVSGTATAGWTFGAG
AALQIPFAMQMAYRENGIGVTQNVLYENQKQIANQFNKAISQIQESLTTTSTALGKLQDVVN
QNAQALNTLVKQLSSNFGAISSVLNDILSRLDKVEAEVQIDRLITGRLQSLQTYVTQQLIRA
AEIRASANLAATKMSECVLGQSKRVDFCGKGYHLMSFPQAAPHGVVFLHVTYVPSQERNFTT
APAICHEGKAYFPREGVFVSNGTSWFITQRNFYSPQIITTDNTFVSGNCDVVIGIINNTVYD
PLQPELDSFKEELDKYFKNHTSPDVDLGDISGINASVVNIQKEIDRLNEVAKNLNESLIDLQ
ELGKYEQ
wt RBD
(SEQ ID NO: 32)
RVQPTESIVRFPNITNLCPFGEVENATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLD
SKVGGNYNYLYRLFRKSNLKPFERDISTEIYQAGSTPCNGVEGFNCYFPLQSYGFQPTNGVG
YQPYRVVVLSFELLHAPATVCGPKKSTNLVKNKCVNF
wt N-terminal signal peptide
(SEQ ID NO: 33)
MFVFLVLLPLVSS
T4-FoldOn Trimerization domain
(SEQ ID NO: 34)
GYIPEAPRDGQAYVRKDGEWVLLSTEL
HRV 3C protease restriction domain
(SEQ ID NO: 35)
LEVLFQGP
HIS8-tag (SEQ ID NO: 36)
HHHHHHHH
Twin-Strep-tag ®
(SEQ ID NO: 37)
SAWSHPQFEKGGGSGGGGSGGSAWSHPQFEK

Ancestral sequence reconstruction has also been performed on the human respiratory syncytial (RS) virus fusion glycoprotein F0.

wt human RS virus fusion glycoprotein F0
(SEQ ID NO: 45)
MELLILKANAITTILTAVTFCFASGQNITEEFYQSTCSAVSKGYLSALRTGWYTSVITIELS
NIKENKCNGTDAKVKLIKQELDKYKNAVTELQLLMQSTPPTNNRARRELPRFMNYTLNNAKK
TNVTLSKKRKRRFLGFLLGVGSAIASGVAVSKVLHLEGEVNKIKSALLSTNKAVVSLSNGVS
VLTSKVLDLKNYIDKQLLPIVNKQSCSISNIETVIEFQQKNNRLLEITREFSVNAGVTTPVS
TYMLTNSELLSLINDMPITNDQKKLMSNNVQIVRQQSYSIMSIIKEEVLAYVVQLPLYGVID
TPCWKLHTSPLCTTNTKEGSNICLTRTDRGWYCDNAGSVSFFPQAETCKVQSNRVFCDTMNS
LTLPSEINLCNVDIFNPKYDCKIMTSKTDVSSSVITSLGAIVSCYGKTKCTASNKNRGIIKT
FSNGCDYVSNKGMDTVSVGNTLYYVNKQEGKSLYVKGEPIINFYDPLVFPSDEFDASISQVN
EKINQSLAFIRKSDELLHNVNAGKSTTNIMITTIIIVIIVILLSLIAVGLLLYCKARSTPVT
LSKDQLSGINNIAFSN

A maturation peptide (RRELPRFMNYTLNNAKKTNVTLSKKRKRR, SEQ ID NO: 46) of the wt human RS virus fusion protein F0 covering two protease cleavage sites is marked with underline in the sequence above and a two-part antigenic site Ø (KNYIDKQLLPIVNKQSC, SEQ ID NO: 47; SNIKENKC, SEQ ID NO: 83) of the wt human RS virus fusion protein F0 is marked in bold in the sequence above.

The RS virus fusion glycoproteins obtained in the ancestral sequence reconstruction, A1 to A5 RSV, and presented below have the following general formula: SP-F2-maturation peptide-F1-GS-T4-GTS-HRV 3C-G-HIS-Twin-Strep. The two-part equivalent of the antigenic site Ø present in the F1 and F2 parts are presented in bold. The wt N-terminal signal peptide SP for these RS virus fusion glycoproteins is MELLILKANAITTILTAVTFCFASG (SEQ ID NO: 48), the T4-FoldOn Trimerization domain is according to SEQ ID NO: 34, the HRV 3C protease restriction domain is according to SEQ ID NO: 35, the HIS₈-tag is according to SEQ ID NO: 36 and the Twin-Strep-Tag® is according to SEQ ID NO: 37.

A1 RSV
(SEQ ID NO: 49)
MELLILKANAITTILTAVTFCFASGQNITEEFYQSTCSAVSRGYLSALRTGWYTSVITIELS
NIKENKCNGTDTKVKLIKQELDKYKNAVTELQLLMQNTPATNNRARREAPRFMNYTLNTTKN
LNVSISKKRKRRFLGFLLGVGSAIASGIAVSKVLHLEGEVNKIKNALLSTNKAVVSLSNGVS
VLTSKVLDLKNYIDKQLLPIVNKQSCRISNIETVIEFQQKNNRLLEITREFSVNAGVTTPLS
TYMLTNSELLSLINDMPITNDQKKLMSSNVQIVRQQSYSIMSIIKEEVLAYVVQLPLYGVID
TPCWKLHTSPLCTTNTKEGSNICLTRTDRGWYCDNAGSVSFFPQAETCKVQSNRVFCDTMNS
LTLPSEVNLCNTDIFNSKYDCKIMTSKTDISSSVITSLGAIVSCYGKTKCTASNKNRGIIKT
FSNGCDYVSNKGVDTVSVGNTLYYVNKLEGKSLYVKGEPIINYYDPLVFPSDEFDASISQVN
EKINQSLAFIRRSDELLGSGYIPEAPRDGQAYVRKDGEWVLLSTFLGTSLEVLFQGPGHHHH
HHHHSAWSHPQFEKGGGSGGGGSGGSAWSHPQFEK
A1 RSV without C-terminal additions
(SEQ ID NO: 50)
MELLILKANAITTILTAVTFCFASGQNITEEFYQSTCSAVSRGYLSALRTGWYTSVITIELS
NIKENKCNGTDTKVKLIKQELDKYKNAVTELQLLMQNTPATNNRARREAPRFMNYTLNTTKN
LNVSISKKRKRRFLGFLLGVGSAIASGIAVSKVLHLEGEVNKIKNALLSTNKAVVSLSNGVS
VLTSKVLDLKNYIDKQLLPIVNKQSCRISNIETVIEFQQKNNRLLEITREFSVNAGVTTPLS
TYMLTNSELLSLINDMPITNDQKKLMSSNVQIVRQQSYSIMSIIKEEVLAYVVQLPLYGVID
TPCWKLHTSPLCTTNTKEGSNICLTRTDRGWYCDNAGSVSFFPQAETCKVQSNRVFCDTMNS
LTLPSEVNLCNTDIFNSKYDCKIMTSKTDISSSVITSLGAIVSCYGKTKCTASNKNRGIIKT
FSNGCDYVSNKGVDTVSVGNTLYYVNKLEGKSLYVKGEPIINYYDPLVFPSDEFDASISQVN
EKINQSLAFIRRSDELL
A1 RSV without N-terminal signal peptide and C-terminal additions
(SEQ ID NO: 51)
QNITEEFYQSTCSAVSRGYLSALRTGWYTSVITIELSNIKENKCNGTDTKVKLIKQELDKYK
NAVTELQLLMQNTPATNNRARREAPRFMNYTLNTTKNLNVSISKKRKRRFLGFLLGVGSAIA
SGIAVSKVLHLEGEVNKIKNALLSTNKAVVSLSNGVSVLTSKVLDLKNYIDKQLLPIVNKQS
CRISNIETVIEFQQKNNRLLEITREFSVNAGVTTPLSTYMLTNSELLSLINDMPITNDQKKL
MSSNVQIVRQQSYSIMSIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNTDIFNSKYDCKIMT
SKTDISSSVITSLGAIVSCYGKTKCTASNKNRGIIKTFSNGCDYVSNKGVDTVSVGNTLYYV
NKLEGKSLYVKGEPIINYYDPLVFPSDEFDASISQVNEKINQSLAFIRRSDELL
A1 RSV-part 1 (F2) without N-terminal signal peptide
(SEQ ID NO: 52)
QNITEEFYQSTCSAVSRGYLSALRTGWYTSVITIELSNIKENKCNGTDTKVKLIKQELDKYK
NAVTELQLLMQNTPATNNRA
A1 RSV-maturation peptide
(SEQ ID NO: 53)
RREAPREMNYTLNTTKNLNVSISKKRKRR
A1 RSV-part 2 (F1)
(SEQ ID NO: 54)
FLGFLLGVGSAIASGIAVSKVLHLEGEVNKIKNALLSTNKAVVSLSNGVSVLTSKVLDLKNY
IDKQLLPIVNKQSCRISNIETVIEFQQKNNRLLEITREFSVNAGVTTPLSTYMLTNSELLSL
INDMPITNDQKKLMSSNVQIVRQQSYSIMSIIKEEVLAYVVQLPLYGVIDTPCWKLHTSPLC
TTNTKEGSNICLTRTDRGWYCDNAGSVSFFPQAETCKVQSNRVFCDTMNSLTLPSEVNLCNT
DIFNSKYDCKIMTSKTDISSSVITSLGAIVSCYGKTKCTASNKNRGIIKTFSNGCDYVSNKG
VDTVSVGNTLYYVNKLEGKSLYVKGEPIINYYDPLVFPSDEFDASISQVNEKINQSLAFIRR
SDELL
A1 RSV-antigenic site Ø
(SEQ ID NO: 83)
SNIKENKC
(SEQ ID NO: 47)
KNYIDKQLLPIVNKQSC
A2 RSV
(SEQ ID NO: 55)
MELLILKANAITTILTAVTFCFASGQNITEEFYQSTCSAVSRGYLSALRTGWYTSVITIELS
NIQKNKCNSTDSKVKLIKQELDRYKNAVTELQLLMQNTPATNNRARRETPRFMGYLLGKKRK
RRFLGFLLGVGSAIASGVAVSKVLHLEGEVNKIKNALLSTNKAVVSLSNGVSVLTSKVLDLK
NYIDKELLPKVNKHSCRISNIETVIEFQQKNNRLLEITREFSVNAGVTTPLSTYMLTNSELL
SLINDMPITNDQKKLMSSNVQIVRQQSYSIMSIVKEEVLAYVVQLPLYGVIDTPCWKLHTSP
LCTTNNKEGSNICLTRTDRGWYCDNAGSVSFFPQAETCKVQSNRVFCDTMNSLTLPTEVNLC
NTDIFNSKYDCKIMTSKTDISSSVITSLGAIVSCYGKTKCTASNKNRGIIKTFSNGCDYVSN
KGVDTVSVGNTLYYVNKLEGKSLYVKGEPIINYYDPLVFPSDEFDASISQVNEKINQSLAFI
RRSDELLGSGYIPEAPRDGQAYVRKDGEWVLLSTFLGTSLEVLFQGPGHHHHHHHHSAWSHP
QFEKGGGSGGGGSGGSAWSHPQFEK
A2 RSV without C-terminal additions
(SEQ ID NO: 56)
MELLILKANAITTILTAVTFCFASGQNITEEFYQSTCSAVSRGYLSALRTGWYTSVITIELS
NIQKNKCNSTDSKVKLIKQELDRYKNAVTELQLLMQNTPATNNRARRETPRFMGYLLGKKRK
RRFLGFLLGVGSAIASGVAVSKVLHLEGEVNKIKNALLSTNKAVVSLSNGVSVLTSKVLDLK
NYIDKELLPKVNKHSCRISNIETVIEFQQKNNRLLEITREFSVNAGVTTPLSTYMLTNSELL
SLINDMPITNDQKKLMSSNVQIVRQQSYSIMSIVKEEVLAYVVQLPLYGVIDTPCWKLHTSP
LCTTNNKEGSNICLTRTDRGWYCDNAGSVSFFPQAETCKVQSNRVFCDTMNSLTLPTEVNLC
NTDIFNSKYDCKIMTSKTDISSSVITSLGAIVSCYGKTKCTASNKNRGIIKTFSNGCDYVSN
KGVDTVSVGNTLYYVNKLEGKSLYVKGEPIINYYDPLVFPSDEFDASISQVNEKINQSLAFI
RRSDELL
A2 RSV without N-terminal signal peptide and C-terminal additions
(SEQ ID NO: 57)
QNITEEFYQSTCSAVSRGYLSALRTGWYTSVITIELSNIQKNKCNSTDSKVKLIKQELDRYK
NAVTELQLLMQNTPATNNRARRETPRFMGYLLGKKRKRRFLGFLLGVGSAIASGVAVSKVLH
LEGEVNKIKNALLSTNKAVVSLSNGVSVLTSKVLDLKNYIDKELLPKVNKHSCRISNIETVI
EFQQKNNRLLEITREFSVNAGVTTPLSTYMLINSELLSLINDMPITNDQKKLMSSNVQIVRQ
QSYSIMSIVKEEVLAYVVQLPLYGVIDTPCWKLHTSPLCTTNNKEGSNICLTRTDRGWYCDN
AGSVSFFPQAETCKVQSNRVFCDTMNSLTLPTEVNLCNTDIFNSKYDCKIMTSKTDISSSVI
TSLGAIVSCYGKTKCTASNKNRGIIKTFSNGCDYVSNKGVDTVSVGNTLYYVNKLEGKSLYV
KGEPIINYYDPLVFPSDEFDASISQVNEKINQSLAFIRRSDELL
A2 RSV-part 1 (F2) without N-terminal signal peptide
(SEQ ID NO: 58)
QNITEEFYQSTCSAVSRGYLSALRTGWYTSVITIELSNIQKNKCNSTDSKVKLIKQELDRYK
NAVTELQLLMQNTPATNNRA
A2 RSV-maturation peptide
(SEQ ID NO: 59)
RRETPRFMGYLLGKKRKRR
A2 RSV-part 2 (F1)
(SEQ ID NO: 60)
FLGFLLGVGSAIASGVAVSKVLHLEGEVNKIKNALLSTNKAVVSLSNGVSVLTSKVLDLKNY
IDKELLPKVNKHSCRISNIETVIEFQQKNNRLLEITREFSVNAGVTTPLSTYMLTNSELLSL
INDMPITNDQKKLMSSNVQIVRQQSYSIMSIVKEEVLAYVVQLPLYGVIDTPCWKLHTSPLC
TTNNKEGSNICLTRTDRGWYCDNAGSVSFFPQAETCKVQSNRVFCDTMNSLTLPTEVNLCNT
DIFNSKYDCKIMTSKTDISSSVITSLGAIVSCYGKTKCTASNKNRGIIKTFSNGCDYVSNKG
VDTVSVGNTLYYVNKLEGKSLYVKGEPIINYYDPLVFPSDEFDASISQVNEKINQSLAFIRR
SDELL
A2 RSV-antigenic site Ø
(SEQ ID NO: 84)
SNIQKNKC
(SEQ ID NO: 61)
KNYIDKELLPKVNKHSC
A3 RSV
(SEQ ID NO: 62)
MELLILKANAITTILTAVTFCFASGNNLTEKFYESTCSVVTRGYKSALRTGWYTSVMTIELS
QSNIENCKCSGTDDSNSLIKQELELYKNAVDELRTLSSDALKNRARRRSPRFLGFILGGFAL
GVGSAVTAGVALAKTIQLEGEVAKIKDALRNTNEAVVSLSNGISVLATAVNDLKNFISKELL
PKINKNSCDISDIKTVIRFQQYNKRLLEVTREFSSNAGITTAVSTYMLTDSELVSLVNDMPV
SSGQKKLMLSNRAIVRRKGFAILSSVNADTLVYVVQLPLFGVIDTPCWKIRSSPLCSNNNDE
YACLARADQGWYCHNAGSISYFPNPEDCEIQNNYVFCDTMNSLTVPTESRECNSNIYTTKYD
CKISTSKTDISTAVLTSLGALVSCYGHTSCTVSNNNKGIIKTLSNGCHYISNKGVDTVSVGN
TVYYLSKLEGKSLYVKGEPIVNNYDPLSFPDDEFDVAISQVNESINQSRSFIKRSDELLGSG
YIPEAPRDGQAYVRKDGEWVLLSTFLGTSLEVLFQGPGHHHHHHHHSAWSHPQFEKGGGSGG
GGSGGSAWSHPQFEK
A3 RSV without C-terminal additions
(SEQ ID NO: 63)
MELLILKANAITTILTAVTFCFASGNNLTEKFYESTCSVVTRGYKSALRTGWYTSVMTIELS
QSNIENCKCSGTDDSNSLIKQELELYKNAVDELRTLSSDALKNRARRRSPRFLGFILGGFAL
GVGSAVTAGVALAKTIQLEGEVAKIKDALRNTNEAVVSLSNGISVLATAVNDLKNFISKELL
PKINKNSCDISDIKTVIRFQQYNKRLLEVTREFSSNAGITTAVSTYMLTDSELVSLVNDMPV
SSGQKKLMLSNRAIVRRKGFAILSSVNADTLVYVVQLPLFGVIDTPCWKIRSSPLCSNNNDE
YACLARADQGWYCHNAGSISYFPNPEDCEIQNNYVFCDTMNSLTVPTESRECNSNIYTTKYD
CKISTSKTDISTAVLTSLGALVSCYGHTSCTVSNNNKGIIKTLSNGCHYISNKGVDTVSVGN
TVYYLSKLEGKSLYVKGEPIVNNYDPLSFPDDEFDVAISQVNESINQSRSFIKRSDELL
A3 RSV without N-terminal signal peptide and C-terminal additions
(SEQ ID NO: 64)
NNLTEKFYESTCSVVTRGYKSALRTGWYTSVMTIELSQSNIENCKCSGTDDSNSLIKQELEL
YKNAVDELRTLSSDALKNRARRRSPRFLGFILGGFALGVGSAVTAGVALAKTIQLEGEVAKI
KDALRNTNEAVVSLSNGISVLATAVNDLKNFISKELLPKINKNSCDISDIKTVIRFQQYNKR
LLEVTREFSSNAGITTAVSTYMLTDSELVSLVNDMPVSSGQKKLMLSNRAIVRRKGFAILSS
VNADTLVYVVQLPLFGVIDTPCWKIRSSPLCSNNNDEYACLARADQGWYCHNAGSISYFPNP
EDCEIQNNYVFCDTMNSLTVPTESRECNSNIYTTKYDCKISTSKTDISTAVLTSLGALVSCY
GHTSCTVSNNNKGIIKTLSNGCHYISNKGVDTVSVGNTVYYLSKLEGKSLYVKGEPIVNNYD
PLSFPDDEFDVAISQVNESINQSRSFIKRSDELL
A3 RSV-part 1 (F2) without N-terminal signal peptide
(SEQ ID NO: 65)
NNLTEKFYESTCSVVTRGYKSALRTGWYTSVMTIELSQSNIENCKCSGTDDSNSLIKQELEL
YKNAVDELRTLSSDALKNRA
A3 RSV-maturation peptide
(SEQ ID NO: 66)
RRRSPRFLGFILG
A3 RSV-part 2 (F1)
(SEQ ID NO: 67)
GFALGVGSAVTAGVALAKTIQLEGEVAKIKDALRNTNEAVVSLSNGISVLATAVNDLKNFIS
KELLPKINKNSCDISDIKTVIRFQQYNKRLLEVTREFSSNAGITTAVSTYMLTDSELVSLVN
DMPVSSGQKKLMLSNRAIVRRKGFAILSSVNADTLVYVVQLPLFGVIDTPCWKIRSSPLCSN
NNDEYACLARADQGWYCHNAGSISYFPNPEDCEIQNNYVFCDTMNSLTVPTESRECNSNIYT
TKYDCKISTSKTDISTAVLTSLGALVSCYGHTSCTVSNNNKGIIKTLSNGCHYISNKGVDTV
SVGNTVYYLSKLEGKSLYVKGEPIVNNYDPLSFPDDEFDVAISQVNESINQSRSFIKRSDEL
L
A3 RSV-antigenic site Ø
(SEQ ID NO: 85)
SNIENCKC
(SEQ ID NO: 68)
KNFISKELLPKINKNSC
A4 RSV
(SEQ ID NO: 69)
MELLILKANAITTILTAVTFCFASGGNLTESYLEETCSTVTRGYKSALRTGWYTNVMTIELS
QGNIENCTCSGTNDSNSLIRQELELTKNALEELKTVSADQLAKEARLRSPRFLGFVLGGIAL
GVATAAAVTAGVALAKTIRLEGEVAAIKNALRNTNEAVSTLSNGVRVLATAVNDLKDFISKE
LLPAINKNSCDISDIKMAISFQQYNRRLLNVVREFSSNAGITPAVSLYMLTDAELVSLVNNM
PTSSGQIKLMLENRAIVRRKGFAILIGVYGDTLVYMVQLPIFGVIDTPCWKVRASPLCSNKD
GEYACLLREDQGWYCQNAGSIAYYPNEEDCEVRNDYVFCDTAASLTVPSESEECNRNIFTTK
YDCKVSTSKTPISTAVLTPLGALVSCYGGVSCSVGNNKKGIIKQLNKGCSYISNKGADTVTI
DNTVYQLSKVEGKSHTIKGEPVVNNYDPLSFPEDEENVALDQVNESIDKSKNFIDKSDELLG
SGYIPEAPRDGQAYVRKDGEWVLLSTFLGTSLEVLFQGPGHHHHHHHHSAWSHPQFEKGGGS
GGGGSGGSAWSHPQFEK
A4 RSV without C-terminal additions
(SEQ ID NO: 70)
MELLILKANAITTILTAVTFCFASGGNLTESYLEETCSTVTRGYKSALRTGWYTNVMTIELS
QGNIENCTCSGTNDSNSLIRQELELTKNALEELKTVSADQLAKEARLRSPRFLGFVLGGIAL
GVATAAAVTAGVALAKTIRLEGEVAAIKNALRNTNEAVSTLSNGVRVLATAVNDLKDFISKE
LLPAINKNSCDISDIKMAISFQQYNRRLLNVVREFSSNAGITPAVSLYMLTDAELVSLVNNM
PTSSGQIKLMLENRAIVRRKGFAILIGVYGDTLVYMVQLPIFGVIDTPCWKVRASPLCSNKD
GEYACLLREDQGWYCQNAGSIAYYPNEEDCEVRNDYVFCDTAASLTVPSESEECNRNIFTTK
YDCKVSTSKTPISTAVLTPLGALVSCYGGVSCSVGNNKKGIIKQLNKGCSYISNKGADTVTI
DNTVYQLSKVEGKSHTIKGEPVVNNYDPLSFPEDEFNVALDQVNESIDKSKNFIDKSDELL
A4 RSV without N-terminal signal peptide and C-terminal additions
(SEQ ID NO: 71)
GNLTESYLEETCSTVTRGYKSALRTGWYTNVMTIELSQGNIENCTCSGTNDSNSLIRQELEL
TKNALEELKTVSADQLAKEARLRSPRFLGFVLGGIALGVATAAAVTAGVALAKTIRLEGEVA
AIKNALRNTNEAVSTLSNGVRVLATAVNDLKDFISKELLPAINKNSCDISDIKMAISFQQYN
RRLLNVVREFSSNAGITPAVSLYMLTDAELVSLVNNMPTSSGQIKLMLENRAIVRRKGFAIL
IGVYGDTLVYMVQLPIFGVIDTPCWKVRASPLCSNKDGEYACLLREDQGWYCQNAGSIAYYP
NEEDCEVRNDYVFCDTAASLTVPSESEECNRNIFTTKYDCKVSTSKTPISTAVLTPLGALVS
CYGGVSCSVGNNKKGIIKQLNKGCSYISNKGADTVTIDNTVYQLSKVEGKSHTIKGEPVVNN
YDPLSFPEDEFNVALDQVNESIDKSKNFIDKSDELL
A4 RSV-part 1 (F2) without N-terminal signal peptide
(SEQ ID NO: 72)
GNLTESYLEETCSTVTRGYKSALRTGWYTNVMTIELSQGNIENCTCSGTNDSNSLIRQELEL
TKNALEELKTVSADQLAKEA
A4 RSV-maturation peptide
(SEQ ID NO: 73)
RLRSPRFLGFVLG
A4 RSV-part 2 (F1)
(SEQ ID NO: 74)
GIALGVATAAAVTAGVALAKTIRLEGEVAAIKNALRNTNEAVSTLSNGVRVLATAVNDLKDF
ISKELLPAINKNSCDISDIKMAISFQQYNRRLLNVVREFSSNAGITPAVSLYMLTDAELVSL
VNNMPTSSGQIKLMLENRAIVRRKGFAILIGVYGDTLVYMVQLPIFGVIDTPCWKVRASPLC
SNKDGEYACLLREDQGWYCQNAGSIAYYPNEEDCEVRNDYVFCDTAASLTVPSESEECNRNI
FTTKYDCKVSTSKTPISTAVLTPLGALVSCYGGVSCSVGNNKKGIIKQLNKGCSYISNKGAD
TVTIDNTVYQLSKVEGKSHTIKGEPVVNNYDPLSFPEDEFNVALDQVNESIDKSKNFIDKSD
ELI
A4 RSV-antigenic site Ø
(SEQ ID NO: 86)
GNIENCTC
(SEQ ID NO: 75)
KDFISKELLPAINKNSC
A5 RSV
(SEQ ID NO: 76)
MELLILKANAITTILTAVTFCFASGGQLDESKLSKIGVIKTKSYELKIYTNSTTSYIVIKLI
PNLSNLKNCSKDTIEEYNKLLNRILSPIKDALERMRNSIQDRKGSGSSRRQKRFFGAIIGGV
ALGVATAAQITAGVALAKAQQNAKNILKLKDAIKKTNEAVQKLQDAAQQLAIAIQAIQDYIN
NEIIPTINQLSCEVAGLKLGIKLSQYYTELTTVFGNNITNPALSPLSIQALYNLEGGNLTEL
LNKLGASNEDLYSLLESGSIKGQIIDVDLEDYLIVLQVKIPTLSEIPGARIQELTSISYNTD
GQEWMAVVPKYVLTRGSLISNIDISDCTITDNSVFCSRNTAYPLPPEMQECLRGNVSKCPYT
KVVGSLVPRFATIDGSIVANCRSITCRCQDPPQTISQDPDQPITIIDQELCKEIQIDGITFR
LSKRLSSTYYRNTNISLGQPVSLDPLDISNELGKVNQSLKESKDYIEKSNEILGSGYIPEAP
RDGQAYVRKDGEWVLLSTFLGTSLEVLFQGPGHHHHHHHHSAWSHPQFEKGGGSGGGGSGGS
AWSHPQFEK
A5 RSV without C-terminal additions
(SEQ ID NO: 77)
MELLILKANAITTILTAVTFCFASGGQLDFSKLSKIGVIKTKSYELKIYTNSTTSYIVIKLI
PNLSNLKNCSKDTIEEYNKLLNRILSPIKDALERMRNSIQDRKGSGSSRRQKRFFGAIIGGV
ALGVATAAQITAGVALAKAQQNAKNILKLKDAIKKTNEAVQKLQDAAQQLAIAIQAIQDYIN
NEIIPTINQLSCEVAGLKLGIKLSQYYTELTTVFGNNITNPALSPLSIQALYNLEGGNLTEL
LNKLGASNEDLYSLLESGSIKGQIIDVDLEDYLIVLQVKIPTLSEIPGARIQELTSISYNTD
GQEWMAVVPKYVLTRGSLISNIDISDCTITDNSVFCSRNTAYPLPPEMQECLRGNVSKCPYT
KVVGSLVPRFATIDGSIVANCRSITCRCQDPPQTISQDPDQPITIIDQELCKEIQIDGITFR
LSKRLSSTYYRNTNISLGQPVSLDPLDISNELGKVNQSLKESKDYIEKSNEIL
A5 RSV without N-terminal signal peptide and C-terminal additions
(SEQ ID NO: 78)
GQLDFSKLSKIGVIKTKSYELKIYTNSTTSYIVIKLIPNLSNLKNCSKDTIEEYNKLLNRIL
SPIKDALERMRNSIQDRKGSGSSRRQKRFFGAIIGGVALGVATAAQITAGVALAKAQQNAKN
ILKLKDAIKKTNEAVQKLQDAAQQLAIAIQAIQDYINNEIIPTINQLSCEVAGLKLGIKLSQ
YYTELTTVFGNNITNPALSPLSIQALYNLFGGNLTELLNKLGASNEDLYSLLESGSIKGQII
DVDLEDYLIVLQVKIPTLSEIPGARIQELTSISYNTDGQEWMAVVPKYVLTRGSLISNIDIS
DCTITDNSVFCSRNTAYPLPPEMQECLRGNVSKCPYTKVVGSLVPRFATIDGSIVANCRSIT
CRCQDPPQTISQDPDQPITIIDQELCKEIQIDGITFRLSKRLSSTYYRNTNISLGQPVSLDP
LDISNELGKVNQSLKESKDYIEKSNEIL
A5 RSV-part 1 (F2) without N-terminal signal peptide
(SEQ ID NO: 79)
GQLDFSKLSKIGVIKTKSYELKIYTNSTTSYIVIKLIPNLSNLKNCSKDTIEEYNKLLNRIL
SPIKDALERMRNSIQDRKGSGSS
A5 RSV-maturation peptide
(SEQ ID NO: 80)
SRRQKRFFGAIIG
A5 RSV-part 2 (F1)
(SEQ ID NO: 81)
GVALGVATAAQITAGVALAKAQQNAKNILKLKDAIKKTNEAVQKLQDAAQQLAIAIQAIQDY
INNEIIPTINQLSCEVAGLKLGIKLSQYYTELTTVFGNNITNPALSPLSIQALYNLEGGNLT
ELLNKLGASNEDLYSLLESGSIKGQIIDVDLEDYLIVLQVKIPTLSEIPGARIQELTSISYN
TDGQEWMAVVPKYVLTRGSLISNIDISDCTITDNSVFCSRNTAYPLPPEMQECLRGNVSKCP
YTKVVGSLVPRFATIDGSIVANCRSITCRCQDPPQTISQDPDQPITIIDQELCKEIQIDGIT
FRISKRLSSTYYRNTNISLGQPVSLDPLDISNELGKVNQSLKESKDYIEKSNEIL
A5 RSV-antigenic site Ø
(SEQ ID NO: 87)
SNLKNCSK
(SEQ ID NO: 82)
QDYINNEIIPTINQLSC

The present invention also relates to a respiratory syncytial (RS) virus fusion glycoprotein F0 comprising an amino acid sequence according to the formula F2-MP-F1. F2 comprises an amino acid sequence selected from the group consisting of SEQ ID NO: 52, 58, 65, 72 and 79, an amino acid sequence selected from the group consisting of SEQ ID NO: 58, 65, 72 and 79 and in which an antigenic site Ø (SEQ ID NO: 84, 85, 86 or 87) is replaced by an antigenic site Ø as defined in SEQ ID NO: 83, and an amino acid sequence having at least 90% sequence identity, preferably at least 95% sequence identity, and more preferably at least 97% sequence identity to any of SEQ ID NO: 52, 58, 65, 72 and 79. MP represents at least one maturation peptide. F1 comprises an amino acid sequence selected from the group consisting of SEQ ID NO: 54, 60, 67, 74 and 81, an amino acid sequence selected from the group consisting of SEQ ID NO: 60, 67, 74 and 81 and in which an antigenic site Ø (SEQ ID NO: 61, 68, 75 or 82) is replaced by an antigenic site Ø as defined in SEQ ID NO: 47, and an amino acid sequence having at least 90% sequence identity, preferably at least 95% sequence identity, and more preferably at least 97% sequence identity to any of SEQ ID NO: 54, 60, 67, 74 and 81.

In an embodiment, the RS virus fusion glycoprotein F0 is according to any of SEQ ID NO: 49-51, 55-57, 62-64, 69-71, 76-78, or an amino acid sequence having at least 90% sequence identity, preferably at least 95% sequence identity, and more preferably at least 97% sequence identity to any of SEQ ID NO: 49-51, 55-57, 62-64, 69-71, 76-78.

In an embodiment, F2 comprises an amino acid sequence selected from the group consisting of SEQ ID NO: 52, 58, 65, 72 and 79 and an amino acid sequence having at least 90% sequence identity, preferably at least 95% sequence identity, and more preferably at least 97% sequence identity to any of SEQ ID NO: 52, 58, 65, 72 and 79. MP comprises an amino acid sequence according to SEQ ID NO: 46. In this embodiment, F1 comprises an amino acid sequence selected from the group consisting of SEQ ID NO: 54, 60, 67, 74 and 81 and an amino acid sequence having at least 90% sequence identity, preferably at least 95% sequence identity, and more preferably at least 97% sequence identity to any of SEQ ID NO: 54, 60, 67, 74 and 81.

In another embodiment, F2 comprises an amino acid sequence selected from the group consisting of SEQ ID NO: 52, 58, 65, 72 and 79 and an amino acid sequence having at least 90% sequence identity, preferably at least 95% sequence identity, and more preferably at least 97% sequence identity to any of SEQ ID NO: 52, 58, 65, 72 and 79. In this embodiment, MP comprises an amino acid sequence according to SEQ ID NO: 46 and an amino acid sequence selected from the group consisting of SEQ ID NO: 53, 59, 66, 73 and 80. In this embodiment, F1 comprises an amino acid sequence selected from the group consisting of SEQ ID NO: 54, 60, 67, 74 and 81 and an amino acid sequence having at least 90% sequence identity, preferably at least 95% sequence identity, and more preferably at least 97% sequence identity to any of SEQ ID NO: 54, 60, 67, 74 and 81.

In a further embodiment, F2 comprises an amino acid sequence selected from the group consisting of SEQ ID NO: 52, 58, 65, 72 and 79 and an amino acid sequence having at least 90% sequence identity, preferably at least 95% sequence identity, and more preferably at least 97% sequence identity to any of SEQ ID NO: 52, 58, 65, 72 and 79. In this embodiment, MP comprises an amino acid sequence selected from the group consisting of SEQ ID NO: 53, 59, 66, 73 and 80. In this embodiment, F1 comprises an amino acid sequence selected from the group consisting of SEQ ID NO: 54, 60, 67, 74 and 81 and an amino acid sequence having at least 90% sequence identity, preferably at least 95% sequence identity, and more preferably at least 97% sequence identity to any of SEQ ID NO: 54, 60, 67, 74 and 81.

In any of the above-described embodiments, the two-part antigenic site Ø present in the F2 and F1 sequences can be replaced by the antigenic site Ø of the wt RS virus fusion glycoprotein F0 as defined in SEQ ID NO: 83 or 47.

The invention also relates to a nucleic acid molecule encoding a RS virus fusion glycoprotein F0 according to above, an expression vector comprising a nucleic acid molecule according to above, and a host cell comprising an expression vector according to above.

Furthermore, the invention relates to a RS virus fusion glycoprotein F0 according to above or a nucleic acid molecule according to above for use as a vaccine or for use in prevention or treatment of a RS virus infection or a RS virus infectious disease, such as bronchiolitis, common colds, or pneumonia.

### EXAMPLES

### Example 1—Ancestral Sequence Reconstruction SARS-CoV-2 Spike Protein

### Ancestral Sequence Reconstruction

The full-length sequence of the SARS-CoV-2 spike protein (SEQ ID NO: 1) was used as input sequence for a Basic Local Alignment Search tool (BLAST) search for homologous sequences. 250 coronavirus spike protein sequences with the highest sequence similarity (excluding single mutants of the SARS-CoV-2 spike protein) were extracted from the BLAST search and aligned using the MUSCLE algorithm in MEGA-X (Edgar 2003; Kumar 2018). The sequences were manually scrutinized for duplicates and single amino acid mutants, which were excluded from the alignment. The spike protein-based phylogenetic tree of the included coronaviruses was constructed using IQ-Tree (Trifinopoulos 2016). The model for construction of the tree was WAG+F+R8 with 1000 bootstrap replication for verification (Whelan 2001). The ancestral sequences were reconstructed using the Maximum Likelihood ancestral inference option in MEGA-X. Three ancestral sequences representing nodes that lie at positions 3, 5 and 6 upstream of the extant sequence in the phylogenic tree were finally selected for analysis.

### Gene Constructs

The ectodomain of the selected ancestral spike protein variants, i.e., positions aligning to residues 14 to 1208 of the wildtype sequence (SEQ ID NO: 1) were reverse translated to nucleotide level using codon tables for expression in human embryonic kidney cells. The final gene constructs were generated by adding the nucleotide sequence of the wildtype spike protein signal peptide in front of the gene and the nucleotide sequence of a GS-linker as well as T4-FoldOn trimerization domain downstream of the gene. The final gene sequence, excluding the trimerization domain, was sequence-optimized for expression in human cells and synthesized in a pMx-series vector by GeneArt services (ThermoFisher Scientific, U.S.).

### Subcloning

The genes were cloned into a poH vector that harbors the SARS-CoV-2 spike protein (pre-fusion stabilized, “HexaPro variant” (Hsieh 2020)), GS-linker and T4-FoldOn trimerization domain under a constitutive cytomegalovirus (CMV) promoter with a C-terminal tag consisting of a GTS-linker, a human rhinovirus (HRV) 3C protease restriction site, a G-linker, a His₈-tag and a Twin-Strep-Tag® (Hsieh 2020). BamHI and SpeI restriction sites were used to replace the HexaPro sequence upstream of the C-terminal tag for the respective ancestral constructs.

### Protein Expression in Mammalian Host Cells

The proteins were expressed in the Expi293 Expression System (ThermoFisher Scientific, U.S.) according to manufacturer's instructions. Human Expi293F cells derived from the HEK 293 cell line were grown in Expi293 expression medium at 37° C. at 115 rpm with 8% CO₂ at 80% humidity. Transient transfections were performed both in small scale (50 mL) in 250 ml non-baffled flasks (Nalgene, U.S.) as well as large scale (1 L) in 2.8 L non-baffled flasks (Nalgene, U.S.). The cells were counted using the CELENA® S Digital Imaging System and then split into 0.8×10⁶ cells/mL the day before transfection and transfected at cell densities between 1.2-1.8×10⁶ cells/mL using 1 μg plasmid DNA/million cells. The DNA was combined with polyethylenimine (PEI) in a 1:1.5 weight ratio, respectively, and incubated at room temperature (˜20-25° C.) for 20 minutes before addition to the cell culture. The transfected cells were left in the incubator under identical growth conditions as described above for three days before protein purification.

### Protein Purification

Cell cultures were harvested three days after transfection by centrifugation at 4000×g at 4° C. and the supernatants were filtered through Rapid-Flow bottle top filters (0.2 μm pore size, Thermo Fischer Scientific, U.S.). The cleared supernatants of the expression cultures were then concentrated to a volume of 100 mL using the Vivaflow 200 Laboratory Cross Flow Cassette (Sartorius, Germany). The concentrated supernatants were incubated overnight at 4° C. in end-over-end rotation with 2 mL Ni-NTA resin (Qiagen, Germany), which had previously been equilibrated twice with 5 mL of wash buffer (20 mM HEPES (4-(2-hydroxyethyl)-1-piperazineethanesulfonic acid) pH 7.5, 200 mM NaCl). The solutions were transferred to an EconoPac chromatography column (Bio-Rad, USA). After the flow-through had emptied by gravity flow, the beads were washed with 5 column volumes (CV) of wash buffer at 4° C. The proteins were eluted from the resin using 4×1 CV of elution buffer (20 mM HEPES pH 7.5, 200 mM NaCl, 250 mM imidazole). The resin was incubated with the elution buffer for 2 minutes before extraction of the proteins. The purity of the elution fractions was confirmed by sodium dodecyl sulphate-polyacrylamide gel electrophoresis (SDS-PAGE) using 4-15% Mini-PROTEAN™ TGX Stain-Free™ Protein Gels (Bio-Rad, U.S.). Eluted protein fractions were concentrated to a volume of about 600 μl in an Amicon Ultra centrifugal spin filter (100 kDa molecular weight cutoff, Merck Group, Germany) that had previously been equilibrated with 15 mL wash buffer.

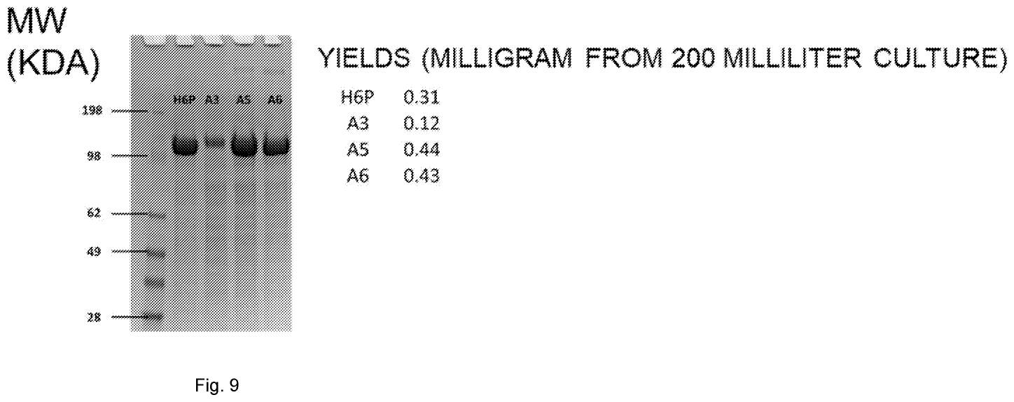

Finally, the proteins were purified by gel filtration using a Superdex 200 increase 10/300 GL column (Cytiva, U.S., formerly GE Health Care, Sweden) in an Agilent 1220 liquid chromatography system using a flow rate of 0.4 mL/min in 100% wash buffer. Elution fractions of 400 μl were collected and their purity was checked by SDS-PAGE (FIG. 9). Fractions corresponding to trimeric spike protein (as assessed by the purification ultraviolet (UV) chromatogram) were collected and concentrated as described above, whereas dimeric and monomeric protein fractions were not collected. Protein concentrations were measured spectrophotometrically using calculated molar extinction coefficients and the purified proteins were used for further studies.

### Cryo-EM Studies

Freshly purified protein sample was applied to cryo-EM grids (R 0.6/1 UltrAuFoil Au 300 mesh) in a Vitrobot Mark IV robot (FEI Thermofisher) and plunge-frozen into liquid ethane. Data were collected in one session on a Krios G3i transmission electron microscope (FEI Thermofisher) operated at 300 kV using EPU software (FEI Thermofisher) at a nominal pixel size of 0.833 Å. For both datasets (A5 and A6), movies with 45 frames were collected with a fluency of 1.11 e−/Å² per frame. The data were processed using CryoSPARC v3.3.1 software. Heterogenous refinement was performed with three classes for A5 and two classes for A6, leading to one significantly superior 3D class that was used for the final 3D reconstruction for A5 and A6 respectively. Homogenous refinement produced a 3D reconstruction at an overall resolution of 2.71 and 2.74 Å for A5 and A6 respectively. These electron density maps clearly show a trimer in the closed pre-fusion state for both A5 and A6 (FIG. 11).

### Results

FIG. 7 illustrates the results of a thermal unfolding assay where the thermal stability of two of the coronavirus spike proteins (A5, A6) was tested and compared to the HexaPro variant of the coronavirus spike protein. The proteins were subjected to reference conditions and denaturing conditions (2 M urea and 2 M guanidinium chloride). The coronavirus spike proteins A5, A6 and HexaPro were transferred to a buffer containing the respective denaturing agent (2 M urea and 2 M guanidine hydrochloride). The coronavirus spike proteins were then transferred to glass capillaries to be measured with nano differential scanning fluorimetry to determine thermal unfolding in a range of temperatures (20° C.-90° C.). The results indicate that the coronavirus spike proteins A5 and A6 perform similarly to the HexaPro variant in this thermal unfolding assay.

HexaPro
(SEQ ID NO: 44)
MFVFLVLLPLVSSQCVNLTTRTQLPPAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNFRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSPIEDLLFNKVTLADAGFIKQYGDCLGDIAARDLICAQKENGLTVLPPLLTDE
MIAQYTSALLAGTITSGWTFGAGPALQIPFPMQMAYRFNGIGVTQNVLYENQKLIANQFNSA
IGKIQDSLSSTPSALGKLQDVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQGSGYIPEAPRDGQAYVRKDGEWVLLSTFLGTS
LEVLFQGPGHHHHHHHHSAWSHPQFEKGGGSGGGGSGGSAWSHPQFEK

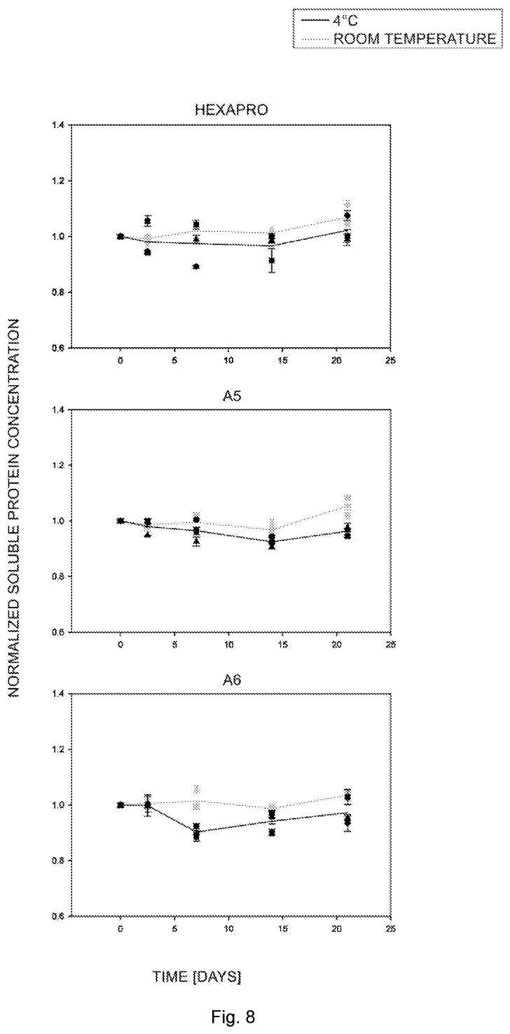

FIG. 8 demonstrates the shelf-life stability of two of the coronavirus spike proteins (A5, A6) stored at 4° C. and room temperature over a 3 week time period by measuring soluble protein concentration over time. After purification of the coronavirus spike proteins, aliquots of the coronavirus spike proteins were stored under these two different temperatures. At timepoints 0, 3, 7, 14 and 21 days a 10 μL sample was transferred to a new tube and spun down to remove aggregates. 6.5 μL was then transferred to a new tube. The sample was measured at 280 nm to determine protein concentration. There was no significant decrease in the concentration of soluble protein over a time period of 3 weeks and the coronavirus proteins A5 and A6 perform similarly to the HexaPro variant in this shelf-stability stability assay.

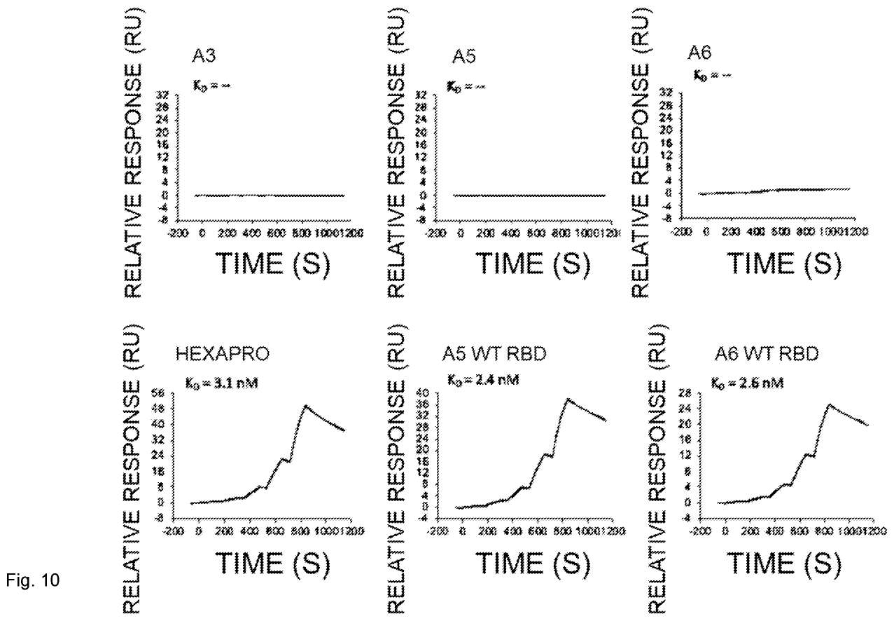

FIG. 10 illustrates that spike proteins generated by the ancestral sequence reconstruction (e.g., A5 and A6) could be used to serve as stable scaffolds to allow further mutations to gain certain properties, such as binding to receptors. The coronavirus spike proteins were tagged with a Strep-tag, which was utilized to dock the coronavirus spike proteins to a SA series S chip (Cytiva #BR100398). 50 nM of the analyte hACE2 receptor was flown over the docked coronavirus spike proteins to observe binding in a BIAcore 8K. The coronavirus spike proteins (A3, A5, A6) did not bind to the hACE2 receptor (FIG. 10, top row). However, replacing the receptor binding domain in these ancestral spike proteins with the receptor binding domain of the wildtype SARS-CoV-2 spike protein (SEQ ID NO: 32) resulted in a gained binding to the hACE2 receptor with similar apparent affinity as the HexaPro variant (FIG. 10, bottom row).

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

### REFERENCES

Ducatez et al., Feasibility of reconstructed ancestral H5N1 influenza viruses for cross-clade protective vaccine development, PNAS (2011) 108(1): 349-354
Edgar, MUSCLE: multiple sequence alignment with high accuracy and high throughput, Nucleic Acids Research (2004) 32(5): 1792-1797
Gaschen et al., Diversity considerations in HIV-1 vaccine selection, Science (2002) 296(5577): 2354-2360
Hsieh et al., Structure-based design of prefusion-stabilized SARS-CoV-2 spikes. Science 2020, 369 (6510), 1501-1505
Kumar et al., MEGA X: Molecular Evolutionary Genetics Analysis across Computing Platforms, Molecular Biology and Evolution (2018) 35(6): 1547-1549
Needleman and Wunsch, A general method applicable to the search for similarities in the amino acid sequence of two proteins, Journal of Molecular Biology (1970) 48(3): 443-453
Selberg et al., Ancestral Sequence Reconstruction: From Chemical Paleogenetics to Maximum Likelihood Algorithms and Beyond, Journal of Molecular Evolution (2021) 89:157-164
Trifinopoulos et al., W-IQ-TREE: A Fast Online Phylogenetic Tool for Maximum Likelihood Analysis, Nucleic Acids Research (2016) 44(W1): W232-W235
Whelan and Goldman, A General Empirical Model of Protein Evolution Derived from Multiple Protein Families Using a Maximum-Likelihood Approach, Molecular Biology and Evolution (2001) 18(5): 691-699

1.-41. (canceled)

42. A protein production method, the method comprising:
providing a plurality of homologous amino acid sequences of a given protein;
determining an amino acid sequence of an ancestral version of the given protein in an ancestral sequence reconstruction method based on the plurality of homologous amino acid sequences of the given protein;
replacing a domain of the amino acid sequence of the ancestral version of the given protein with a corresponding domain derived from an amino acid sequence of the given protein or a homologous version thereof; and
producing a protein comprising the amino acid sequence obtained by replacing the domain of the amino acid sequence of the ancestral version of the given protein with the corresponding domain derived from the amino acid sequence of the given protein or the homologous version thereof.

43. The method according to claim 42, wherein
replacing the domain comprises replacing the domain of the amino acid sequence of the ancestral version of the given protein with a corresponding domain derived from an amino acid sequence selected among the plurality of homologous amino acid sequences of the given protein; and
producing the protein comprises producing the protein comprising the amino acid sequence obtained by replacing the domain of the amino acid sequence of the ancestral version of the protein with the corresponding domain derived from the amino acid sequence selected among the plurality of homologous amino acid sequences of the given protein.

44. The method according to claim 42, wherein providing the plurality of homologous amino acid sequences comprises:
providing an amino acid sequence of the given protein; and
identifying a plurality of amino acid sequences having a sequence identity of at least 40% with the provided amino acid sequence of the given protein.

45. The method according to claim 42, wherein providing the plurality of homologous amino acid sequences comprises:
providing an amino acid sequence of the given protein; and
identifying, in a protein database, the N amino acid sequences having highest sequence identity with the provided amino acid sequence of the given protein, wherein N is at least 25.

46. The method according to 44, wherein replacing the domain comprises replacing the domain of the amino acid sequence of the ancestral version of the given protein with a corresponding domain derived from the provided amino acid sequence of the given protein.

47. The method according to any one of claim 44, further comprising removing, from the identified amino acid sequences, any duplicate amino acid sequences.

48. The method according to any one of claim 44, further comprising removing, from the identified amino acid sequences, any amino acid sequence being a single amino acid mutant of the amino acid sequence of the given protein or of the plurality of homologous amino acid sequences of the given protein.

49. The method according to claim 42, wherein determining the amino acid sequence comprises determining the amino acid sequence of a node of a phylogenetic tree generated in the ancestral sequence reconstruction method based on the plurality of homologous amino acid sequences of the given protein.

50. The method according to claim 42, wherein
the domain of the amino acid sequence of the ancestral version of the given protein is a domain of a plurality of M consecutive amino acids of the amino acid sequence of the ancestral version of the given protein;
the corresponding domain derived from the amino acid sequence of the given protein or the homologous version thereof is a corresponding domain of a plurality of N consecutive amino acids of the amino acid sequence of the given protein or the homologous version thereof; and
each of M, N is at least 5.

51. The method according to claim 42, wherein replacing the domain comprises replacing a receptor binding domain, a host binding domain, an antigenic domain or an immunogenic domain of the amino acid sequence of the ancestral version of the given protein with a corresponding receptor binding domain, a corresponding host binding domain, a corresponding antigenic domain or a corresponding immunogenic domain derived from the amino acid sequence of the given protein or the homologous version thereof.

52. The method according to claim 51, wherein replacing the domain comprises replacing a receptor binding domain or a host binding domain of the amino acid sequence of the ancestral version of the given protein with a corresponding receptor binding domain or a corresponding host binding domain derived from the amino acid sequence of the given protein or the homologous version thereof.

53. The method according to claim 52, wherein replacing the domain comprises replacing a receptor binding domain of the amino acid sequence of the ancestral version of the given protein with a corresponding receptor binding domain derived from the amino acid sequence of the given protein or the homologous version thereof.

54. The method according to claim 52, wherein replacing the domain comprises replacing a host binding domain of the amino acid sequence of the ancestral version of the given protein with a corresponding host binding domain derived from the amino acid sequence of the given protein or the homologous version thereof, wherein the host binding domain is configured to bind to a macromolecule present on a cell surface of an animal cell.

55. The method according to claim 42, wherein producing the protein comprises:
determining a nucleotide sequence encoding the amino acid sequence obtained by replacing the domain of the amino acid sequence of the ancestral version of the given protein with the corresponding domain derived from the amino acid sequence of the given protein or the homologous version thereof;
expressing a gene construct comprising the determined nucleotide sequence in a host cell comprising the gene construct; and
isolating the protein from the host cell or from a culture medium, in which the host cell is cultured.

56. The method according to claim 42, further comprising performing a structural study of the produced protein by X-ray crystallography or cryo-electron (CE) microscopy.

57. The method according to claim 42, wherein
providing the plurality of homologous amino acid sequences comprises providing a plurality of homologous amino acid sequences of a pathogen protein;
determining the amino acid sequence comprises determining an amino acid sequence of an ancestral version of the pathogen protein in an ancestral sequence reconstruction method based on the plurality of homologous amino acid sequences of the pathogen protein;
replacing the domain comprises replacing a domain of the amino acid sequence of the ancestral version of the pathogen protein with a corresponding domain derived from an amino acid sequence of the pathogen protein or a homologous version thereof; and
producing the protein comprises producing an antigenic protein comprising the amino acid sequence obtained by replacing the domain of the amino acid sequence of the ancestral pathogen protein with the corresponding domain derived from the amino acid sequence of the pathogen protein or the homologous version thereof.

58. The method according to claim 57, wherein the antigenic protein is an antigenic virus protein.

59. A coronavirus spike protein comprising an amino acid sequence according to the formula Seq1-RBD-Seq2, wherein
Seq1 comprises an amino acid sequence selected from the group consisting of SEQ ID NO: 5, 15, 25;
RBD represents a receptor binding domain; and
Seq2 comprises an amino acid sequence selected from the group consisting of SEQ ID NO: 8, 18, 28.

60. The coronavirus spike protein according to claim 59, wherein the receptor binding domain comprises an amino acid sequence selected from the group consisting of SEQ ID NO: 7, 17, 27, 32.

61. The coronavirus spike protein according to claim 59, wherein Seq1 comprises an amino acid sequence selected from the group consisting of SEQ ID NO: 6, 16, 26.

62. The coronavirus spike protein according to claim 59, wherein
Seq1 comprises an amino acid sequence according to SEQ ID NO: 25; and
Seq2 comprises an amino acid sequence according to SEQ ID NO: 28.

63. The coronavirus spike protein according to claim 62, wherein Seq1 comprises an amino acid sequence according to SEQ ID NO: 26.

64. The coronavirus spike protein according to claim 62, wherein the receptor binding domain comprises an amino acid sequence selected from the group consisting of SEQ ID NO: 27, and 32.

65. The coronavirus spike protein according to claim 64, wherein the coronavirus spike protein comprises an amino acid sequence selected from the group consisting of SEQ ID NO: 22, 23, 24, 29, 30, and 31.

66. The coronavirus spike protein according to claim 59, wherein
Seq1 comprises an amino acid sequence according to SEQ ID NO: 5; and
Seq2 comprises an amino acid sequence according to SEQ ID NO: 8.

67. The coronavirus spike protein according to claim 66, wherein Seq1 comprises an amino acid sequence according to SEQ ID NO: 6.

68. The coronavirus spike protein according to claim 66, wherein the receptor binding domain comprises an amino acid sequence selected from the group consisting of SEQ ID NO: 7, and 32.

69. The coronavirus spike protein according to claim 68, wherein the coronavirus spike protein comprises an amino acid sequence selected from the group consisting of SEQ ID NO: 2, 3, 4, 9, 10, and 11.

70. The coronavirus spike protein according to claim 59, wherein
Seq1 comprises an amino acid sequence according to SEQ ID NO: 15; and
Seq2 comprises an amino acid sequence according to SEQ ID NO: 18.

71. The coronavirus spike protein according to claim 70, wherein Seq1 comprises an amino acid sequence according to SEQ ID NO: 16.

72. The coronavirus spike protein according to claim 70, wherein the receptor binding domain comprises an amino acid sequence selected from the group consisting of SEQ ID NO: 17, and 32.

73. The coronavirus spike protein according to claim 72, wherein the coronavirus spike protein comprises an amino acid sequence selected from the group consisting of SEQ ID NO: 12, 13, 14, 19, 20, and 21.

74. The coronavirus spike protein according to claim 59, wherein the coronavirus spike protein comprises an amino acid sequence selected from the group consisting of SEQ ID NO: 2, 3, 4, 9, 10, 11, 12, 13, 14, 19, 20, 21, 22, 23, 24, 29, 30, and 31.

75. The coronavirus spike protein according to claim 74, wherein the coronavirus spike protein comprises an amino acid sequence selected from the group consisting of SEQ ID NO: 2, 3, 4, 12, 13, 14, 22, 23, and 24.

76. The coronavirus spike protein according to claim 75, wherein the coronavirus spike protein comprises an amino acid sequence selected from the group consisting of SEQ ID NO: 9, 10, 11, 19, 20, 21, 29, 30, and 31.

77. The coronavirus spike protein according to claim 59, wherein the coronavirus spike protein comprises multiple amino acid sequences according to the formula Seq1-RBD-Seq2.

78. A nucleic acid molecule encoding a coronavirus spike protein according to claim 59.

79. An expression vector comprising a nucleic acid molecule according to claim 78.

80. A host cell comprising an expression vector according to claim 79.


SEQUENCE LISTING (87 sequences)

SEQ ID NO: 1 - protein, 1273 aa, synthetic construct
  REGION 1..1273: note = SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1262 aa, synthetic construct
  REGION 1..1262: note = Ancestral spike protein
MFVFLVLLPLVSSQCVNLTGRTPLNPNYTNSSQRGVYYPDTIFRSDTLVLSQGYFLPFYSNVSWYYSLTTNNAATKRFDNPILDFKDGIYFAATEKSNIIRGWIFGTTLDNTSQSLLIVNNATNVIIKVCNFQFCYDPYLSGYYGHNNKTWSIREFAVYSSYANCTFEYVSKSFMLDISGKGGLFNTLREFVFRNVDGYFKIYSKYTPVNLNRGLPTGFSVLQPLVELPVGINITKFRTLLTIHRGDPMPNNGWTAFSAAYFVGYLKPRTFMLKYNENGTITDAVDCALDPLSETKCTLKSLTVQKGIYQTSNFRVQPTQSVVRFPNITNLCPFHKVFNATRFPSVYAWERTKISDCVADYTVFYNSTSFSTFKCYGVSPSKLIDLCFTSVYADTFLIRFSEVRQVAPGQTGVIADYNYKLPDDFTGCVIAWNTAKQDVGNYFYRSHRSTKLKPFERDLSSQDENGVRTLSTYDFNPNVPLEYQATRVVVLSFELLNAPATVCGPKLSTQLVKNQCVNFNFNGLKGTGVLTDSSKRFQSFQQFGRDASDFTDSVRDPQTLEILDITPCSFGGVSVITPGTNTSSEVAVLYQDVNCTDVPTAIHADQLTPAWRIYSTGTNVFQTQAGCLIGAEHVNASYECDIPIGAGICASYHTASILRSTSQKAIVAYTMSLGAENSIAYANNSIAIPTNFSISVTTEVMPVSMAKTSVDCTMYICGDSIECSNLLLQYGSFCTQLNRALTGIAIEQDKNTQEVFAQVKQIYKTPPIKDFGGFNFSQILPDPSKPSKRSFIEDLLFNKVTLADAGFIKQYGDCLGDISARDLICAQKFNGLTVLPPLLTDEMIAAYTAALISGTATAGWTFGAGAALQIPFAMQMAYRFNGIGVTQNVLYENQKLIANQFNSAIGKIQESLTSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPSQEKNFTTAPAICHEGKAHFPREGVFVSNGTHWFVTQRNFYEPQIITTDNTFVSGNCDVVIGIINNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 3 - protein, 1182 aa, synthetic construct
  REGION 1..1182: note = Ancestral spike protein
MFVFLVLLPLVSSQCVNLTGRTPLNPNYTNSSQRGVYYPDTIFRSDTLVLSQGYFLPFYSNVSWYYSLTTNNAATKRFDNPILDFKDGIYFAATEKSNIIRGWIFGTTLDNTSQSLLIVNNATNVIIKVCNFQFCYDPYLSGYYGHNNKTWSIREFAVYSSYANCTFEYVSKSFMLDISGKGGLFNTLREFVFRNVDGYFKIYSKYTPVNLNRGLPTGFSVLQPLVELPVGINITKFRTLLTIHRGDPMPNNGWTAFSAAYFVGYLKPRTFMLKYNENGTITDAVDCALDPLSETKCTLKSLTVQKGIYQTSNFRVQPTQSVVRFPNITNLCPFHKVFNATRFPSVYAWERTKISDCVADYTVFYNSTSFSTFKCYGVSPSKLIDLCFTSVYADTFLIRFSEVRQVAPGQTGVIADYNYKLPDDFTGCVIAWNTAKQDVGNYFYRSHRSTKLKPFERDLSSQDENGVRTLSTYDFNPNVPLEYQATRVVVLSFELLNAPATVCGPKLSTQLVKNQCVNFNFNGLKGTGVLTDSSKRFQSFQQFGRDASDFTDSVRDPQTLEILDITPCSFGGVSVITPGTNTSSEVAVLYQDVNCTDVPTAIHADQLTPAWRIYSTGTNVFQTQAGCLIGAEHVNASYECDIPIGAGICASYHTASILRSTSQKAIVAYTMSLGAENSIAYANNSIAIPTNFSISVTTEVMPVSMAKTSVDCTMYICGDSIECSNLLLQYGSFCTQLNRALTGIAIEQDKNTQEVFAQVKQIYKTPPIKDFGGFNFSQILPDPSKPSKRSFIEDLLFNKVTLADAGFIKQYGDCLGDISARDLICAQKFNGLTVLPPLLTDEMIAAYTAALISGTATAGWTFGAGAALQIPFAMQMAYRFNGIGVTQNVLYENQKLIANQFNSAIGKIQESLTSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPSQEKNFTTAPAICHEGKAHFPREGVFVSNGTHWFVTQRNFYEPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 4 - protein, 1169 aa, synthetic construct
  REGION 1..1169: note = Ancestral spike protein
QCVNLTGRTPLNPNYTNSSQRGVYYPDTIFRSDTLVLSQGYFLPFYSNVSWYYSLTTNNAATKRFDNPILDFKDGIYFAATEKSNIIRGWIFGTTLDNTSQSLLIVNNATNVIIKVCNFQFCYDPYLSGYYGHNNKTWSIREFAVYSSYANCTFEYVSKSFMLDISGKGGLFNTLREFVFRNVDGYFKIYSKYTPVNLNRGLPTGFSVLQPLVELPVGINITKFRTLLTIHRGDPMPNNGWTAFSAAYFVGYLKPRTFMLKYNENGTITDAVDCALDPLSETKCTLKSLTVQKGIYQTSNFRVQPTQSVVRFPNITNLCPFHKVFNATRFPSVYAWERTKISDCVADYTVFYNSTSFSTFKCYGVSPSKLIDLCFTSVYADTFLIRFSEVRQVAPGQTGVIADYNYKLPDDFTGCVIAWNTAKQDVGNYFYRSHRSTKLKPFERDLSSQDENGVRTLSTYDFNPNVPLEYQATRVVVLSFELLNAPATVCGPKLSTQLVKNQCVNFNFNGLKGTGVLTDSSKRFQSFQQFGRDASDFTDSVRDPQTLEILDITPCSFGGVSVITPGTNTSSEVAVLYQDVNCTDVPTAIHADQLTPAWRIYSTGTNVFQTQAGCLIGAEHVNASYECDIPIGAGICASYHTASILRSTSQKAIVAYTMSLGAENSIAYANNSIAIPTNFSISVTTEVMPVSMAKTSVDCTMYICGDSIECSNLLLQYGSFCTQLNRALTGIAIEQDKNTQEVFAQVKQIYKTPPIKDFGGFNFSQILPDPSKPSKRSFIEDLLFNKVTLADAGFIKQYGDCLGDISARDLICAQKFNGLTVLPPLLTDEMIAAYTAALISGTATAGWTFGAGAALQIPFAMQMAYRFNGIGVTQNVLYENQKLIANQFNSAIGKIQESLTSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPSQEKNFTTAPAICHEGKAHFPREGVFVSNGTHWFVTQRNFYEPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 5 - protein, 301 aa, synthetic construct
  REGION 1..301: note = N-terminal part of ancestral spike protein
QCVNLTGRTPLNPNYTNSSQRGVYYPDTIFRSDTLVLSQGYFLPFYSNVSWYYSLTTNNAATKRFDNPILDFKDGIYFAATEKSNIIRGWIFGTTLDNTSQSLLIVNNATNVIIKVCNFQFCYDPYLSGYYGHNNKTWSIREFAVYSSYANCTFEYVSKSFMLDISGKGGLFNTLREFVFRNVDGYFKIYSKYTPVNLNRGLPTGFSVLQPLVELPVGINITKFRTLLTIHRGDPMPNNGWTAFSAAYFVGYLKPRTFMLKYNENGTITDAVDCALDPLSETKCTLKSLTVQKGIYQTSNF

SEQ ID NO: 6 - protein, 314 aa, synthetic construct
  REGION 1..314: note = N-terminal part of ancestral spike protein
MFVFLVLLPLVSSQCVNLTGRTPLNPNYTNSSQRGVYYPDTIFRSDTLVLSQGYFLPFYSNVSWYYSLTTNNAATKRFDNPILDFKDGIYFAATEKSNIIRGWIFGTTLDNTSQSLLIVNNATNVIIKVCNFQFCYDPYLSGYYGHNNKTWSIREFAVYSSYANCTFEYVSKSFMLDISGKGGLFNTLREFVFRNVDGYFKIYSKYTPVNLNRGLPTGFSVLQPLVELPVGINITKFRTLLTIHRGDPMPNNGWTAFSAAYFVGYLKPRTFMLKYNENGTITDAVDCALDPLSETKCTLKSLTVQKGIYQTSNF

SEQ ID NO: 7 - protein, 205 aa, synthetic construct
  REGION 1..205: note = Receptor binding domain of ancestral spike protein
RVQPTQSVVRFPNITNLCPFHKVFNATRFPSVYAWERTKISDCVADYTVFYNSTSFSTFKCYGVSPSKLIDLCFTSVYADTFLIRFSEVRQVAPGQTGVIADYNYKLPDDFTGCVIAWNTAKQDVGNYFYRSHRSTKLKPFERDLSSQDENGVRTLSTYDFNPNVPLEYQATRVVVLSFELLNAPATVCGPKLSTQLVKNQCVNF

SEQ ID NO: 8 - protein, 663 aa, synthetic construct
  REGION 1..663: note = C-terminal part of ancestral spike protein
NFNGLKGTGVLTDSSKRFQSFQQFGRDASDFTDSVRDPQTLEILDITPCSFGGVSVITPGTNTSSEVAVLYQDVNCTDVPTAIHADQLTPAWRIYSTGTNVFQTQAGCLIGAEHVNASYECDIPIGAGICASYHTASILRSTSQKAIVAYTMSLGAENSIAYANNSIAIPTNFSISVTTEVMPVSMAKTSVDCTMYICGDSIECSNLLLQYGSFCTQLNRALTGIAIEQDKNTQEVFAQVKQIYKTPPIKDFGGFNFSQILPDPSKPSKRSFIEDLLFNKVTLADAGFIKQYGDCLGDISARDLICAQKFNGLTVLPPLLTDEMIAAYTAALISGTATAGWTFGAGAALQIPFAMQMAYRFNGIGVTQNVLYENQKLIANQFNSAIGKIQESLTSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPSQEKNFTTAPAICHEGKAHFPREGVFVSNGTHWFVTQRNFYEPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 9 - protein, 1280 aa, synthetic construct
  REGION 1..1280: note = Ancestral spike protein
MFVFLVLLPLVSSQCVNLTGRTPLNPNYTNSSQRGVYYPDTIFRSDTLVLSQGYFLPFYSNVSWYYSLTTNNAATKRFDNPILDFKDGIYFAATEKSNIIRGWIFGTTLDNTSQSLLIVNNATNVIIKVCNFQFCYDPYLSGYYGHNNKTWSIREFAVYSSYANCTFEYVSKSFMLDISGKGGLFNTLREFVFRNVDGYFKIYSKYTPVNLNRGLPTGFSVLQPLVELPVGINITKFRTLLTIHRGDPMPNNGWTAFSAAYFVGYLKPRTFMLKYNENGTITDAVDCALDPLSETKCTLKSLTVQKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLKGTGVLTDSSKRFQSFQQFGRDASDFTDSVRDPQTLEILDITPCSFGGVSVITPGTNTSSEVAVLYQDVNCTDVPTAIHADQLTPAWRIYSTGTNVFQTQAGCLIGAEHVNASYECDIPIGAGICASYHTASILRSTSQKAIVAYTMSLGAENSIAYANNSIAIPTNFSISVTTEVMPVSMAKTSVDCTMYICGDSIECSNLLLQYGSFCTQLNRALTGIAIEQDKNTQEVFAQVKQIYKTPPIKDFGGFNFSQILPDPSKPSKRSFIEDLLFNKVTLADAGFIKQYGDCLGDISARDLICAQKFNGLTVLPPLLTDEMIAAYTAALISGTATAGWTFGAGAALQIPFAMQMAYRFNGIGVTQNVLYENQKLIANQFNSAIGKIQESLTSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPSQEKNFTTAPAICHEGKAHFPREGVFVSNGTHWFVTQRNFYEPQIITTDNTFVSGNCDVVIGIINNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 10 - protein, 1200 aa, synthetic construct
  REGION 1..1200: note = Ancestral spike protein
MFVFLVLLPLVSSQCVNLTGRTPLNPNYTNSSQRGVYYPDTIFRSDTLVLSQGYFLPFYSNVSWYYSLTTNNAATKRFDNPILDFKDGIYFAATEKSNIIRGWIFGTTLDNTSQSLLIVNNATNVIIKVCNFQFCYDPYLSGYYGHNNKTWSIREFAVYSSYANCTFEYVSKSFMLDISGKGGLFNTLREFVFRNVDGYFKIYSKYTPVNLNRGLPTGFSVLQPLVELPVGINITKFRTLLTIHRGDPMPNNGWTAFSAAYFVGYLKPRTFMLKYNENGTITDAVDCALDPLSETKCTLKSLTVQKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLKGTGVLTDSSKRFQSFQQFGRDASDFTDSVRDPQTLEILDITPCSFGGVSVITPGTNTSSEVAVLYQDVNCTDVPTAIHADQLTPAWRIYSTGTNVFQTQAGCLIGAEHVNASYECDIPIGAGICASYHTASILRSTSQKAIVAYTMSLGAENSIAYANNSIAIPTNFSISVTTEVMPVSMAKTSVDCTMYICGDSIECSNLLLQYGSFCTQLNRALTGIAIEQDKNTQEVFAQVKQIYKTPPIKDFGGFNFSQILPDPSKPSKRSFIEDLLFNKVTLADAGFIKQYGDCLGDISARDLICAQKFNGLTVLPPLLTDEMIAAYTAALISGTATAGWTFGAGAALQIPFAMQMAYRFNGIGVTQNVLYENQKLIANQFNSAIGKIQESLTSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPSQEKNFTTAPAICHEGKAHFPREGVFVSNGTHWFVTQRNFYEPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 11 - protein, 1187 aa, synthetic construct
  REGION 1..1187: note = Ancestral spike protein
QCVNLTGRTPLNPNYTNSSQRGVYYPDTIFRSDTLVLSQGYFLPFYSNVSWYYSLTTNNAATKRFDNPILDFKDGIYFAATEKSNIIRGWIFGTTLDNTSQSLLIVNNATNVIIKVCNFQFCYDPYLSGYYGHNNKTWSIREFAVYSSYANCTFEYVSKSFMLDISGKGGLFNTLREFVFRNVDGYFKIYSKYTPVNLNRGLPTGFSVLQPLVELPVGINITKFRTLLTIHRGDPMPNNGWTAFSAAYFVGYLKPRTFMLKYNENGTITDAVDCALDPLSETKCTLKSLTVQKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLKGTGVLTDSSKRFQSFQQFGRDASDFTDSVRDPQTLEILDITPCSFGGVSVITPGTNTSSEVAVLYQDVNCTDVPTAIHADQLTPAWRIYSTGTNVFQTQAGCLIGAEHVNASYECDIPIGAGICASYHTASILRSTSQKAIVAYTMSLGAENSIAYANNSIAIPTNFSISVTTEVMPVSMAKTSVDCTMYICGDSIECSNLLLQYGSFCTQLNRALTGIAIEQDKNTQEVFAQVKQIYKTPPIKDFGGFNFSQILPDPSKPSKRSFIEDLLFNKVTLADAGFIKQYGDCLGDISARDLICAQKFNGLTVLPPLLTDEMIAAYTAALISGTATAGWTFGAGAALQIPFAMQMAYRFNGIGVTQNVLYENQKLIANQFNSAIGKIQESLTSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPSQEKNFTTAPAICHEGKAHFPREGVFVSNGTHWFVTQRNFYEPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 12 - protein, 1258 aa, synthetic construct
  REGION 1..1258: note = Ancestral spike protein
MFVFLVLLPLVSSTAQEGTCGTISNKTPPNMNQFSSSRRGVYYPDDIFRSDVLHLTQDYFLPFNSNVTRYLSLNADSNRIVRFDNPILPFGDGIYFAATEKSNVIRGWIFGSTLDNTSQSAIIVNNSTHIIIKVCNFQLCDDPMFTVSRGQHYKTWVYTNARNCTYEYVSKSFQLDVSEKNGNFKHLREFVFKNVDGFLHVYSAYEPIDLARGLPSGFSVLKPILKLPLGINITSFRVVMTMFSPTTSNWLAESAAYFVGYLKPTTFMLKFNENGTITDAVDCSQDPLSELKCTLKSFNVEKGIYQTSNFRVSPTQEVVRFPNITNLCPFDKVFNATRFPSVYAWERTKISDCVADYTVLYNSTSFSTFKCYGVSPSKLIDLCFTSVYADTFLIRSSEVRQVAPGQTGVIADYNYKLPDDFTGCVIAWNTAKQDAGNYYYRSHRKTKLKPFERDLSNSDENGVRTLSTYDFNPNVPIEYQATRVVVLSFELLNAPATVCGPKLSTQLVKNQCVNFNFNGLKGTGVLTDSSKRFQSFQQFGRDASDFTDSVRDPQTLEILDISPCSFGGVSVITPGTNASSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYANNSIAIPTNFSISVTTEVMPVSMAKTSVDCTMYICGDSQECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVAGNCDVVIGIINNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 13 - protein, 1178 aa, synthetic construct
  REGION 1..1178: note = Ancestral spike protein
MFVFLVLLPLVSSTAQEGTCGTISNKTPPNMNQFSSSRRGVYYPDDIFRSDVLHLTQDYFLPFNSNVTRYLSLNADSNRIVRFDNPILPFGDGIYFAATEKSNVIRGWIFGSTLDNTSQSAIIVNNSTHIIIKVCNFQLCDDPMFTVSRGQHYKTWVYTNARNCTYEYVSKSFQLDVSEKNGNFKHLREFVFKNVDGFLHVYSAYEPIDLARGLPSGFSVLKPILKLPLGINITSFRVVMTMFSPTTSNWLAESAAYFVGYLKPTTFMLKFNENGTITDAVDCSQDPLSELKCTLKSFNVEKGIYQTSNFRVSPTQEVVRFPNITNLCPFDKVFNATRFPSVYAWERTKISDCVADYTVLYNSTSFSTFKCYGVSPSKLIDLCFTSVYADTFLIRSSEVRQVAPGQTGVIADYNYKLPDDFTGCVIAWNTAKQDAGNYYYRSHRKTKLKPFERDLSNSDENGVRTLSTYDFNPNVPIEYQATRVVVLSFELLNAPATVCGPKLSTQLVKNQCVNFNFNGLKGTGVLTDSSKRFQSFQQFGRDASDFTDSVRDPQTLEILDISPCSFGGVSVITPGTNASSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYANNSIAIPTNFSISVTTEVMPVSMAKTSVDCTMYICGDSQECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVAGNCDVVIGIINNTVYDPLQPELDSFKEELDKYFKNHTSPDVDLGDISGINASVVNIQKEIDRLNEVAKNLNESLIDLQELGKYEQ

SEQ ID NO: 14 - protein, 1165 aa, synthetic construct
  REGION 1..1165: note = Ancestral spike protein
TAQEGTCGTISNKTPPNMNQFSSSRRGVYYPDDIFRSDVLHLTQDYFLPFNSNVTRYLSLNADSNRIVRFDNPILPFGDGIYFAATEKSNVIRGWIFGSTLDNTSQSAIIVNNSTHIIIKVCNFQLCDDPMFTVSRGQHYKTWVYTNARNCTYEYVSKSFQLDVSEKNGNFKHLREFVFKNVDGFLHVYSAYEPIDLARGLPSGFSVLKPILKLPLGINITSFRVVMTMFSPTTSNWLAESAAYFVGYLKPTTFMLKFNENGTITDAVDCSQDPLSELKCTLKSFNVEKGIYQTSNFRVSPTQEVVRFPNITNLCPFDKVFNATRFPSVYAWERTKISDCVADYTVLYNSTSFSTFKCYGVSPSKLIDLCFTSVYADTFLIRSSEVRQVAPGQTGVIADYNYKLPDDFTGCVIAWNTAKQDAGNYYYRSHRKTKLKPFERDLSNSDENGVRTLSTYDFNPNVPIEYQATRVVVLSFELLNAPATVCGPKLSTQLVKNQCVNFNFNGLKGTGVLTDSSKRFQSFQQFGRDASDFTDSVRDPQTLEILDISPCSFGGVSVITPGTNASSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYANNSIAIPTNFSISVTTEVMPVSMAKTSVDCTMYICGDSQECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVAGNCDVVIGIINNTVYDPLQPELDSFKEELDKYFKNHTSPDVDLGDISGINASVVNIQKEIDRLNEVAKNLNESLIDLQELGKYEQ

SEQ ID NO: 15 - protein, 297 aa, synthetic construct
  REGION 1..297: note = N-terminal part of ancestral spike protein
TAQEGTCGTISNKTPPNMNQFSSSRRGVYYPDDIFRSDVLHLTQDYFLPFNSNVTRYLSLNADSNRIVRFDNPILPFGDGIYFAATEKSNVIRGWIFGSTLDNTSQSAIIVNNSTHIIIKVCNFQLCDDPMFTVSRGQHYKTWVYTNARNCTYEYVSKSFQLDVSEKNGNFKHLREFVFKNVDGFLHVYSAYEPIDLARGLPSGFSVLKPILKLPLGINITSFRVVMTMFSPTTSNWLAESAAYFVGYLKPTTFMLKFNENGTITDAVDCSQDPLSELKCTLKSFNVEKGIYQTSNF

SEQ ID NO: 16 - protein, 310 aa, synthetic construct
  REGION 1..310: note = N-terminal part of ancestral spike protein
MFVFLVLLPLVSSTAQEGTCGTISNKTPPNMNQFSSSRRGVYYPDDIFRSDVLHLTQDYFLPFNSNVTRYLSLNADSNRIVRFDNPILPFGDGIYFAATEKSNVIRGWIFGSTLDNTSQSAIIVNNSTHIIIKVCNFQLCDDPMFTVSRGQHYKTWVYTNARNCTYEYVSKSFQLDVSEKNGNFKHLREFVFKNVDGFLHVYSAYEPIDLARGLPSGFSVLKPILKLPLGINITSFRVVMTMFSPTTSNWLAESAAYFVGYLKPTTFMLKFNENGTITDAVDCSQDPLSELKCTLKSFNVEKGIYQTSNF

SEQ ID NO: 17 - protein, 205 aa, synthetic construct
  REGION 1..205: note = Receptor binding domain of ancestral spike protein
RVSPTQEVVRFPNITNLCPFDKVFNATRFPSVYAWERTKISDCVADYTVLYNSTSFSTFKCYGVSPSKLIDLCFTSVYADTFLIRSSEVRQVAPGQTGVIADYNYKLPDDFTGCVIAWNTAKQDAGNYYYRSHRKTKLKPFERDLSNSDENGVRTLSTYDFNPNVPIEYQATRVVVLSFELLNAPATVCGPKLSTQLVKNQCVNF

SEQ ID NO: 18 - protein, 663 aa, synthetic construct
  REGION 1..663: note = C-terminal part of ancestral spike protein
NFNGLKGTGVLTDSSKRFQSFQQFGRDASDFTDSVRDPQTLEILDISPCSFGGVSVITPGTNASSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYANNSIAIPTNFSISVTTEVMPVSMAKTSVDCTMYICGDSQECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVAGNCDVVIGIINNTVYDPLQPELDSFKEELDKYFKNHTSPDVDLGDISGINASVVNIQKEIDRLNEVAKNLNESLIDLQELGKYEQ

SEQ ID NO: 19 - protein, 1276 aa, synthetic construct
  REGION 1..1276: note = Ancestral spike protein
MFVFLVLLPLVSSTAQEGTCGTISNKTPPNMNQFSSSRRGVYYPDDIFRSDVLHLTQDYFLPFNSNVTRYLSLNADSNRIVRFDNPILPFGDGIYFAATEKSNVIRGWIFGSTLDNTSQSAIIVNNSTHIIIKVCNFQLCDDPMFTVSRGQHYKTWVYTNARNCTYEYVSKSFQLDVSEKNGNFKHLREFVFKNVDGFLHVYSAYEPIDLARGLPSGFSVLKPILKLPLGINITSFRVVMTMFSPTTSNWLAESAAYFVGYLKPTTFMLKFNENGTITDAVDCSQDPLSELKCTLKSFNVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLKGTGVLTDSSKRFQSFQQFGRDASDFTDSVRDPQTLEILDISPCSFGGVSVITPGTNASSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYANNSIAIPTNFSISVTTEVMPVSMAKTSVDCTMYICGDSQECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVAGNCDVVIGIINNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 20 - protein, 1196 aa, synthetic construct
  REGION 1..1196: note = Ancestral spike protein
MFVFLVLLPLVSSTAQEGTCGTISNKTPPNMNQFSSSRRGVYYPDDIFRSDVLHLTQDYFLPFNSNVTRYLSLNADSNRIVRFDNPILPFGDGIYFAATEKSNVIRGWIFGSTLDNTSQSAIIVNNSTHIIIKVCNFQLCDDPMFTVSRGQHYKTWVYTNARNCTYEYVSKSFQLDVSEKNGNFKHLREFVFKNVDGFLHVYSAYEPIDLARGLPSGFSVLKPILKLPLGINITSFRVVMTMFSPTTSNWLAESAAYFVGYLKPTTFMLKFNENGTITDAVDCSQDPLSELKCTLKSFNVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLKGTGVLTDSSKRFQSFQQFGRDASDFTDSVRDPQTLEILDISPCSFGGVSVITPGTNASSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYANNSIAIPTNFSISVTTEVMPVSMAKTSVDCTMYICGDSQECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVAGNCDVVIGIINNTVYDPLQPELDSFKEELDKYFKNHTSPDVDLGDISGINASVVNIQKEIDRLNEVAKNLNESLIDLQELGKYEQ

SEQ ID NO: 21 - protein, 1183 aa, synthetic construct
  REGION 1..1183: note = Ancestral spike protein
TAQEGTCGTISNKTPPNMNQFSSSRRGVYYPDDIFRSDVLHLTQDYFLPFNSNVTRYLSLNADSNRIVRFDNPILPFGDGIYFAATEKSNVIRGWIFGSTLDNTSQSAIIVNNSTHIIIKVCNFQLCDDPMFTVSRGQHYKTWVYTNARNCTYEYVSKSFQLDVSEKNGNFKHLREFVFKNVDGFLHVYSAYEPIDLARGLPSGFSVLKPILKLPLGINITSFRVVMTMFSPTTSNWLAESAAYFVGYLKPTTFMLKFNENGTITDAVDCSQDPLSELKCTLKSFNVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLKGTGVLTDSSKRFQSFQQFGRDASDFTDSVRDPQTLEILDISPCSFGGVSVITPGTNASSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYANNSIAIPTNFSISVTTEVMPVSMAKTSVDCTMYICGDSQECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVAGNCDVVIGIINNTVYDPLQPELDSFKEELDKYFKNHTSPDVDLGDISGINASVVNIQKEIDRLNEVAKNLNESLIDLQELGKYEQ

SEQ ID NO: 22 - protein, 1277 aa, synthetic construct
  REGION 1..1277: note = Ancestral spike protein
MFVFLVLLPLVSSTAQEGTCGTLSNKSPPNMTQFSSSRRGVYYPDDIFRSDVLHLTQDYFLPFNSNVTRYLSLNSDSDRIVRFDNPIIPFGDGVYFAATEKSNVIRGWIFGSTLDNTSQSAIIMNNSTHIVIRVCNFQLCDDPMFAVSRPTGQHYKTWIYTNARNCTYEYVSKSFQLDVSEKPGNFKHLREFVFKNVDGFLHVYSGYEPIDVARGLPSGFSVLKPIFKLPLGINITNFRVIMTMFSPTTSNWGAEAAAYFVGYLKPTTFMLKFDENGTITDAVDCSQDPLSELKCTVKSFNVEKGIYQTSNFRVSPTKEVVRFPNITNLCPFGEVFNATTFPSVYAWERTRISDCVADYSVLYNSTSFSTFKCYGVSPTKLNDLCFSSVYADSFVVKGDDVRQIAPGQTGVIADYNYKLPDDFTGCVIAWNTANLDATSTGNYNYYYRSLRHGKLKPFERDISNVPFSPEGKPCTPPAFNCYRPLNTYGFNPTVGIGYQPYRVVVLSFELLNAPATVCGPKLSTELVKNQCVNFNFNGLTGTGVLTDSSKRFQPFQQFGRDVSDFTDSVRDPKTLEILDISPCSFGGVSVITPGTNTSSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYSNNTIAIPTNFSISVTTEVMPVSMAKTSVDCTMYICGDSTECSNLLLQYGSFCTQLNRALSGIAVEQDKNTREVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 23 - protein, 1197 aa, synthetic construct
  REGION 1..1197: note = Ancestral spike protein
MFVFLVLLPLVSSTAQEGTCGTLSNKSPPNMTQFSSSRRGVYYPDDIFRSDVLHLTQDYFLPFNSNVTRYLSLNSDSDRIVRFDNPIIPFGDGVYFAATEKSNVIRGWIFGSTLDNTSQSAIIMNNSTHIVIRVCNFQLCDDPMFAVSRPTGQHYKTWIYTNARNCTYEYVSKSFQLDVSEKPGNFKHLREFVFKNVDGFLHVYSGYEPIDVARGLPSGFSVLKPIFKLPLGINITNFRVIMTMFSPTTSNWGAEAAAYFVGYLKPTTFMLKFDENGTITDAVDCSQDPLSELKCTVKSFNVEKGIYQTSNFRVSPTKEVVRFPNITNLCPFGEVFNATTFPSVYAWERTRISDCVADYSVLYNSTSFSTFKCYGVSPTKLNDLCFSSVYADSFVVKGDDVRQIAPGQTGVIADYNYKLPDDFTGCVIAWNTANLDATSTGNYNYYYRSLRHGKLKPFERDISNVPFSPEGKPCTPPAFNCYRPLNTYGFNPTVGIGYQPYRVVVLSFELLNAPATVCGPKLSTELVKNQCVNFNFNGLTGTGVLTDSSKRFQPFQQFGRDVSDFTDSVRDPKTLEILDISPCSFGGVSVITPGTNTSSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYSNNTIAIPTNFSISVTTEVMPVSMAKTSVDCTMYICGDSTECSNLLLQYGSFCTQLNRALSGIAVEQDKNTREVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 24 - protein, 1184 aa, synthetic construct
  REGION 1..1184: note = Ancestral spike protein
TAQEGTCGTLSNKSPPNMTQFSSSRRGVYYPDDIFRSDVLHLTQDYFLPFNSNVTRYLSLNSDSDRIVRFDNPIIPFGDGVYFAATEKSNVIRGWIFGSTLDNTSQSAIIMNNSTHIVIRVCNFQLCDDPMFAVSRPTGQHYKTWIYTNARNCTYEYVSKSFQLDVSEKPGNFKHLREFVFKNVDGFLHVYSGYEPIDVARGLPSGFSVLKPIFKLPLGINITNFRVIMTMFSPTTSNWGAEAAAYFVGYLKPTTFMLKFDENGTITDAVDCSQDPLSELKCTVKSFNVEKGIYQTSNFRVSPTKEVVRFPNITNLCPFGEVFNATTFPSVYAWERTRISDCVADYSVLYNSTSFSTFKCYGVSPTKLNDLCFSSVYADSFVVKGDDVRQIAPGQTGVIADYNYKLPDDFTGCVIAWNTANLDATSTGNYNYYYRSLRHGKLKPFERDISNVPFSPEGKPCTPPAFNCYRPLNTYGFNPTVGIGYQPYRVVVLSFELLNAPATVCGPKLSTELVKNQCVNFNFNGLTGTGVLTDSSKRFQPFQQFGRDVSDFTDSVRDPKTLEILDISPCSFGGVSVITPGTNTSSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYSNNTIAIPTNFSISVTTEVMPVSMAKTSVDCTMYICGDSTECSNLLLQYGSFCTQLNRALSGIAVEQDKNTREVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 25 - protein, 299 aa, synthetic construct
  REGION 1..299: note = N-terminal part of ancestral spike protein
TAQEGTCGTLSNKSPPNMTQFSSSRRGVYYPDDIFRSDVLHLTQDYFLPFNSNVTRYLSLNSDSDRIVRFDNPIIPFGDGVYFAATEKSNVIRGWIFGSTLDNTSQSAIIMNNSTHIVIRVCNFQLCDDPMFAVSRPTGQHYKTWIYTNARNCTYEYVSKSFQLDVSEKPGNFKHLREFVFKNVDGFLHVYSGYEPIDVARGLPSGFSVLKPIFKLPLGINITNFRVIMTMFSPTTSNWGAEAAAYFVGYLKPTTFMLKFDENGTITDAVDCSQDPLSELKCTVKSFNVEKGIYQTSNF

SEQ ID NO: 26 - protein, 312 aa, synthetic construct
  REGION 1..312: note = N-terminal part of ancestral spike protein
MFVFLVLLPLVSSTAQEGTCGTLSNKSPPNMTQFSSSRRGVYYPDDIFRSDVLHLTQDYFLPFNSNVTRYLSLNSDSDRIVRFDNPIIPFGDGVYFAATEKSNVIRGWIFGSTLDNTSQSAIIMNNSTHIVIRVCNFQLCDDPMFAVSRPTGQHYKTWIYTNARNCTYEYVSKSFQLDVSEKPGNFKHLREFVFKNVDGFLHVYSGYEPIDVARGLPSGFSVLKPIFKLPLGINITNFRVIMTMFSPTTSNWGAEAAAYFVGYLKPTTFMLKFDENGTITDAVDCSQDPLSELKCTVKSFNVEKGIYQTSNF

SEQ ID NO: 27 - protein, 222 aa, synthetic construct
  REGION 1..222: note = Receptor binding domain of ancestral spike protein
RVSPTKEVVRFPNITNLCPFGEVFNATTFPSVYAWERTRISDCVADYSVLYNSTSFSTFKCYGVSPTKLNDLCFSSVYADSFVVKGDDVRQIAPGQTGVIADYNYKLPDDFTGCVIAWNTANLDATSTGNYNYYYRSLRHGKLKPFERDISNVPFSPEGKPCTPPAFNCYRPLNTYGFNPTVGIGYQPYRVVVLSFELLNAPATVCGPKLSTELVKNQCVNF

SEQ ID NO: 28 - protein, 663 aa, synthetic construct
  REGION 1..663: note = C-terminal part of ancestral spike protein
NFNGLTGTGVLTDSSKRFQPFQQFGRDVSDFTDSVRDPKTLEILDISPCSFGGVSVITPGTNTSSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYSNNTIAIPTNFSISVTTEVMPVSMAKTSVDCTMYICGDSTECSNLLLQYGSFCTQLNRALSGIAVEQDKNTREVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 29 - protein, 1278 aa, synthetic construct
  REGION 1..1278: note = Ancestral spike protein
MFVFLVLLPLVSSTAQEGTCGTLSNKSPPNMTQFSSSRRGVYYPDDIFRSDVLHLTQDYFLPFNSNVTRYLSLNSDSDRIVRFDNPIIPFGDGVYFAATEKSNVIRGWIFGSTLDNTSQSAIIMNNSTHIVIRVCNFQLCDDPMFAVSRPTGQHYKTWIYTNARNCTYEYVSKSFQLDVSEKPGNFKHLREFVFKNVDGFLHVYSGYEPIDVARGLPSGFSVLKPIFKLPLGINITNFRVIMTMFSPTTSNWGAEAAAYFVGYLKPTTFMLKFDENGTITDAVDCSQDPLSELKCTVKSFNVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTDSSKRFQPFQQFGRDVSDFTDSVRDPKTLEILDISPCSFGGVSVITPGTNTSSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYSNNTIAIPTNFSISVTTEVMPVSMAKTSVDCTMYICGDSTECSNLLLQYGSFCTQLNRALSGIAVEQDKNTREVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 30 - protein, 1198 aa, synthetic construct
  REGION 1..1198: note = Ancestral spike protein
MFVFLVLLPLVSSTAQEGTCGTLSNKSPPNMTQFSSSRRGVYYPDDIFRSDVLHLTQDYFLPFNSNVTRYLSLNSDSDRIVRFDNPIIPFGDGVYFAATEKSNVIRGWIFGSTLDNTSQSAIIMNNSTHIVIRVCNFQLCDDPMFAVSRPTGQHYKTWIYTNARNCTYEYVSKSFQLDVSEKPGNFKHLREFVFKNVDGFLHVYSGYEPIDVARGLPSGFSVLKPIFKLPLGINITNFRVIMTMFSPTTSNWGAEAAAYFVGYLKPTTFMLKFDENGTITDAVDCSQDPLSELKCTVKSFNVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTDSSKRFQPFQQFGRDVSDFTDSVRDPKTLEILDISPCSFGGVSVITPGTNTSSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYSNNTIAIPTNFSISVTTEVMPVSMAKTSVDCTMYICGDSTECSNLLLQYGSFCTQLNRALSGIAVEQDKNTREVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 31 - protein, 1185 aa, synthetic construct
  REGION 1..1185: note = Ancestral spike protein
TAQEGTCGTLSNKSPPNMTQFSSSRRGVYYPDDIFRSDVLHLTQDYFLPFNSNVTRYLSLNSDSDRIVRFDNPIIPFGDGVYFAATEKSNVIRGWIFGSTLDNTSQSAIIMNNSTHIVIRVCNFQLCDDPMFAVSRPTGQHYKTWIYTNARNCTYEYVSKSFQLDVSEKPGNFKHLREFVFKNVDGFLHVYSGYEPIDVARGLPSGFSVLKPIFKLPLGINITNFRVIMTMFSPTTSNWGAEAAAYFVGYLKPTTFMLKFDENGTITDAVDCSQDPLSELKCTVKSFNVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTDSSKRFQPFQQFGRDVSDFTDSVRDPKTLEILDISPCSFGGVSVITPGTNTSSEVAVLYQDVNCTDVPTAIHADQLTPAWRVYSTGVNVFQTQAGCLIGAEHVNASYECDIPIGAGICASYHTASTLRSTGQKSIVAYTMSLGAENSIAYSNNTIAIPTNFSISVTTEVMPVSMAKTSVDCTMYICGDSTECSNLLLQYGSFCTQLNRALSGIAVEQDKNTREVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 32 - protein, 223 aa, synthetic construct
  REGION 1..223: note = Receptor binding domain of SARS-CoV-2 spike protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 33 - protein, 13 aa, synthetic construct
  REGION 1..13: note = N-terminal signal petide
MFVFLVLLPLVSS

SEQ ID NO: 34 - protein, 27 aa, synthetic construct
  REGION 1..27: note = T4-FoldOn Trimerization domain
GYIPEAPRDGQAYVRKDGEWVLLSTFL

SEQ ID NO: 35 - protein, 8 aa, synthetic construct
  REGION 1..8: note = HRV C3 protease restriction domain
LEVLFQGP

SEQ ID NO: 36 - protein, 8 aa, synthetic construct
  REGION 1..8: note = HIS8-tag
HHHHHHHH

SEQ ID NO: 37 - protein, 31 aa, synthetic construct
  REGION 1..31: note = Twin Strep-tag
SAWSHPQFEKGGGSGGGGSGGSAWSHPQFEK

SEQ ID NO: 38 - DNA, 3789 bp, synthetic construct
  misc_feature 1..3789: note = Ancestral spike protein
  source 1..3789: mol_type = other DNA
atgttcgtgtttctggtgctgctgcctctggtgtccagccagtgtgtgaatctgaccggcagaacccctctgaaccccaactacaccaacagcagccagcggggcgtgtactaccccgacaccatctttagaagcgacaccctggtgctgagccagggctacttcctgcctttctacagcaacgtgtcctggtactacagcctgaccaccaacaacgccgccaccaagagattcgacaaccccatcctggacttcaaggacggcatctactttgccgccaccgagaagtccaacatcatcagaggctggatcttcggcaccacactggacaacacaagccagagcctgctgatcgtgaacaacgccaccaacgtgatcatcaaagtgtgcaacttccagttctgctacgacccctacctgagcggctactacggccacaacaacaagacctggtccatccgcgagttcgccgtgtacagcagctacgccaattgcaccttcgagtacgtgtccaagagcttcatgctggacatcagcggcaaaggcggcctgttcaataccctgcgcgagtttgtgttcagaaacgtggacggctacttcaagatctactccaagtacacccctgtgaacctgaaccggggcctgcctacaggcttttctgttctgcagcccctggtggaactgcccgtgggcatcaacatcaccaagttcagaaccctgctgaccatccacagaggcgaccccatgcctaacaatggctggaccgcctttagcgccgcctacttcgtgggctacctgaagcctcggacctttatgctgaagtacaacgagaacggcaccatcaccgacgccgtggattgtgcccttgatcctctgagcgagacaaagtgcaccctgaagtccctgacagtgcagaagggcatctaccagaccagcaacttccgggtgcagcccacacagtctgtcgtgcggttccccaatatcaccaatctgtgccccttccacaaggtgttcaacgccacaagattccccagcgtgtacgcctgggagcgcaccaagatctctgattgcgtggccgactacaccgtgttctacaacagcacctccttcagcaccttcaagtgctacggcgtgtcccctagcaagctgatcgacctgtgcttcacctctgtgtacgccgacaccttcctgatccggttcagcgaagtgcgacaggtggcacctggacagacaggcgtgatcgccgattacaactacaagctgcccgacgacttcaccggctgtgtgatcgcctggaataccgccaagcaggacgtgggcaactacttctacagaagccacagaagcaccaagctgaagcccttcgagagggacctgagcagccaggatgagaatggcgtgcggaccctgagcacctacgacttcaaccccaatgtgcccctggaatatcaggccaccagagtggtggtgctgtccttcgagctgctgaatgcccctgccacagtgtgtggccctaagctgtctacccagctggtcaagaaccagtgcgtgaacttcaacttcaacggcctgaaaggcaccggcgtgctgaccgatagcagcaagagatttcagagcttccagcagttcggccgggacgccagcgatttcacagacagcgttagagatccccagacactggaaatcctggatatcaccccttgcagcttcggcggagtgtccgtgatcacccctggcacaaataccagctctgaggtggccgtgctgtaccaggatgtgaactgcaccgatgtgcccacagccatccatgccgatcagctgacacccgcctggcggatctatagcaccggcaccaatgtgtttcagacccaagccggctgtctgattggagccgagcatgtgaatgccagctacgagtgcgacatccctatcggagccggcatctgcgccagctaccacacagcctctatcctgagatccaccagccagaaagccatcgtggcctacacaatgtctctgggcgccgagaactctatcgcctacgccaacaacagcattgctatccccaccaacttctccatcagcgtgaccaccgaagtgatgcccgtgtccatggccaagaccagcgtggactgcaccatgtacatctgcggcgacagcatcgagtgcagcaacctgctgctgcagtacggcagcttctgcacccagctgaatagagccctgaccggaatcgccatcgagcaggacaagaacacccaagaggtgttcgcccaagtgaagcagatctacaagacccctcctatcaaggacttcggcggcttcaatttcagccagatcctgccagatccaagcaagcccagcaagcggagcttcatcgaggacctgctgttcaacaaagtgacactggccgacgccggcttcatcaagcagtatggcgattgcctgggcgacatctccgccagggatctgatttgcgcccagaagttcaacggactgaccgtgctgcctcctctgctgacagatgagatgatcgccgcctataccgccgctctgatctctggaacagccaccgccggatggacatttggagctggtgcagctctgcagatccccttcgctatgcagatggcctaccggttcaatggcatcggcgtgacccagaatgtgctgtacgagaaccagaagctgattgccaaccagttcaacagcgccatcggcaagatccaagagagcctgacctctaccgccagcgctctgggaaagctgcaggacgtcgtgaaccagaacgcccaggctctgaacaccctcgtgaagcagctgagcagcaatttcggcgccatcagcagcgtgctgaacgacatcctgagccggctggataaggtggaagccgaggtgcagatcgaccggctgattacaggcagactgcagagcctccagacctacgtgacacagcagctgatcagagccgccgagattagagcctctgccaatctggccgccacaaagatgagcgagtgtgtgctgggccagagcaagagagtggacttttgcggcaagggctaccacctgatgagcttccctcagtctgctcctcacggcgtggtgtttctgcacgtgacatacgtgcccagccaagagaagaacttcaccaccgctccagccatctgccacgagggcaaagcccactttcctagagaaggcgtgttcgtgtccaacggcacccattggttcgtgactcagcggaacttctacgagccccagatcatcaccaccgacaacaccttcgtgtccggcaactgcgacgtggtcatcggcatcatcaacaataccgtgtacgacccactgcagcccgagctggacagcttcaaagaggaactggacaagtactttaagaaccacacaagccccgacgtggacctgggcgatatcagcggaatcaatgccagcgtggtcaacatccagaaagagatcgataggctgaacgaggtggccaagaacctgaatgagtccctgattgacctgcaagagctggggaagtacgagcaaggatctggctatattcccgaggctcctagagatggccaggcctacgttagaaaggacggcgagtgggtcctgctgagcacctttcttggaactagtctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtga

SEQ ID NO: 39 - DNA, 3777 bp, synthetic construct
  misc_feature 1..3777: note = Ancestral spike protein
  source 1..3777: mol_type = other DNA
atgttcgtgtttctggtgctgctgcctctggtgtccagcacagctcaagagggcacctgtggcaccatcagcaacaagacccctccaaacatgaaccagttcagcagcagcagacggggcgtgtactaccccgacgacatcttcagatccgacgtgctgcatctgacccaggactacttcctgcctttcaacagcaacgtgaccagatacctgagcctgaacgccgacagcaaccggatcgtcagattcgacaaccccatcctgccattcggcgacggcatctattttgccgccaccgagaagtccaacgtgatcagaggctggatcttcggcagcaccctggacaataccagccagagcgccatcatcgtgaacaacagcacccacatcatcatcaaagtgtgcaacttccagctgtgcgacgaccccatgttcaccgtgtctagaggccagcactacaagacctgggtgtacacaaacgcccggaactgcacctacgagtacgtgtccaagagctttcagctggacgtgtccgagaagaacggcaacttcaagcacctgagggaatttgtgttcaagaacgtggacggcttcctgcacgtgtacagcgcctacgagcctatcgacctggctagaggactgcctagcggcttctctgtgctgaagcccatcctgaagctgcccctgggcatcaacatcaccagcttcagagtcgtgatgaccatgttcagccccaccaccagcaattggctggccgaaagcgccgcctacttcgtgggatacctgaagccaaccaccttcatgctgaagttcaacgagaacggcacaatcaccgacgccgtggactgttctcaggaccctctgagcgagctgaagtgcaccctgaagtccttcaacgtggaaaagggcatctaccagaccagcaacttccgggtgtcccctacacaagaggtcgtgcggttccccaatatcaccaatctgtgccccttcgacaaggtgttcaacgccaccagatttcccagcgtgtacgcctgggagcgcaccaagatctctgattgcgtggccgactacaccgtgctgtacaactccacctccttcagcaccttcaagtgctacggcgtgtccccaagcaagctgatcgacctgtgcttcacctctgtgtacgccgacaccttcctgatccggtctagcgaagtgcgacaggtggcacctggacagacaggcgtgatcgccgattacaactacaagctgcccgacgacttcaccggctgtgtgatcgcctggaataccgccaaacaggacgccggcaactactactacagaagccacagaaagaccaagctcaagcccttcgagcgggacctgagcaactccgatgagaatggcgtgcggaccctgtccacctacgacttcaaccccaatgtgcccatcgagtaccaggccaccagagtggtggtgctgtccttcgagctgctgaatgcccctgccacagtgtgtggccctaagctgtctacccagctggtcaagaaccagtgcgtgaacttcaacttcaacggcctgaaaggcaccggcgtgctgaccgatagcagcaagagattccagagcttccagcagttcggcagggacgccagcgatttcacagacagcgtcagagatccccagacactggaaatcctggacatcagcccttgcagcttcggcggagtgtctgtgatcacccctggcacaaatgccagctctgaagtggctgtgctgtaccaggacgtgaactgtaccgatgtgcccacagccatccacgccgatcaactgacaccagcttggcgggtgtactctaccggtgtcaacgtgttccagacacaagccggctgtctgattggagccgaacacgtgaacgccagctacgagtgcgacatccctatcggagctggaatctgcgcctcctaccacacagccagcacactgagaagcaccggccagaaatccatcgtggcctacacaatgtctctgggcgccgagaactctatcgcctacgccaacaactccattgctatccccaccaacttctccatcagcgtgaccaccgaagtgatgcccgtgtccatggccaagacctccgtggattgcaccatgtacatctgcggcgacagccaagagtgcagcaacctgctgctccagtacggcagcttctgcacccagctgaatagagccctgaccggaatcgccatcgagcaggacaagaacacccaagaggtgttcgcccaagtgaagcagatgtataagacccctgccatcaaggacttcggcggcttcaatttcagccagatcctgcctgatcctagcaagcccaccaagcggagcttcatcgaggacctgctgttcaacaaagtgaccctggccgacgccggctttatgaagcagtatggcgagtgcctgggcgacatctctgccagggatctgatttgcgcccagaagtttaacggactgaccgtgctgcctcctctgctgacagatgagatgatcgccgcctataccgccgcactggtgtctggtactgctaccgccggatggacatttggagctggtgccgctctgcagatccccttcgctatgcagatggcctacagattcaacggcatcggcgtgacccagaacgtgctgtatgagaaccagaagcagatcgccaatcagttcaacaaggccatcagtcagatccaagagagcctgaccaccacaagcacagccctgggaaagctgcaggacgtggtcaaccagaatgcccaggctctgaacaccctggtcaagcagctgagcagcaatttcggcgccatcagcagcgtgctgaacgacatcctgagccggctggataaggtggaagccgaggtgcagatcgaccggctgattacaggcagactgcagagcctgcagacctacgtgacacagcagctgatcagagccgccgagattagagcctctgccaatctggccgccaccaagatgagcgagtgtgtgctgggccagagcaagagagtggacttttgcggcaagggctaccacctgatgagcttccctcaagccgctcctcacggcgtggtgtttctgcacgtgacatacgtgccaagccaagagcggaacttcaccaccgctccagccatttgccacgagggcaaagcctactttccccgcgaaggcgtgttcgtgtctaacggcacctcctggttcatcacccagaggaacttctacagccctcagatcatcaccaccgacaacaccttcgtggccggcaattgcgacgtcgtgatcggcatcatcaacaataccgtgtacgaccctctgcagcccgagctggacagcttcaaagaggaactggacaagtacttcaagaatcacacaagccccgacgtggacctgggcgatatcagcggaatcaatgccagcgtcgtgaacatccagaaagagatcgacagactgaacgaggtggccaagaacctgaacgagtccctgattgacctgcaagagctggggaagtacgagcaaggatctggctatattcccgaggctcctagagatggccaggcctacgttagaaaggacggcgagtgggtcctgctgagcacctttcttggaactagtctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtga

SEQ ID NO: 40 - DNA, 3834 bp, synthetic construct
  misc_feature 1..3834: note = Ancestral spike protein
  source 1..3834: mol_type = other DNA
atgttcgtgtttctggtgctgctgcccctggtgtcctctacagctcaagagggcacatgtggcaccctgagcaacaagagccctcctaacatgacccagttcagcagctctcggagaggcgtgtactaccccgacgacatcttcagatccgacgtgctgcatctgacccaggactacttcctgcctttcaacagcaacgtgaccagatacctgagcctgaacagcgacagcgaccggatcgtcagattcgacaaccctatcatccccttcggcgacggggtgtactttgccgccaccgagaagtccaacgtgatcagaggctggatcttcggcagcaccctggacaataccagccagagcgccatcatcatgaacaacagcacccacatcgtgattcgcgtgtgcaacttccagctgtgcgacgaccctatgttcgccgtgtctagacctaccggccagcactacaagacctggatctacaccaacgccagaaactgcacctacgagtacgtgtccaagagctttcagctggacgtgtccgagaagcccggcaacttcaaacacctgagggaatttgtgttcaagaacgtggacggcttcctgcacgtgtacagcggctacgagcctatcgatgtggccagaggactgcctagcggcttctctgtgctgaagcccatcttcaagctgcctctgggcatcaacatcaccaacttcagagtgatcatgaccatgttcagccccaccaccagcaactggggagctgaagccgccgcttacttcgtgggctacctgaagcctaccaccttcatgctgaagttcgacgagaacggcaccatcaccgacgccgtggactgtagccaagatcctctgagcgagctgaagtgcaccgtgaagtccttcaacgtggaaaagggcatctaccagaccagcaatttccgggtgtcccctaccaaagaagtcgtgcggttccccaatatcaccaatctgtgccctttcggcgaggtgttcaacgccaccacctttccatctgtgtacgcctgggagagaacccggatcagcgattgcgtggccgattacagcgtgctgtacaactccaccagcttctccaccttcaagtgctacggcgtgtcacccaccaagctgaacgacctgtgcttcagcagcgtgtacgccgacagctttgtggtcaagggcgacgatgtgcggcagattgctcctggacagacaggcgtgatcgccgactacaactacaagctgcccgacgacttcaccggctgtgtgatcgcctggaacaccgccaatctggatgccaccagcaccggcaactacaattactactacagaagcctgcggcacggcaagctgaaacccttcgagagggacatctccaacgtgccattcagccctgagggcaagccttgtacacctccagccttcaactgttacagacccctgaacacctacggcttcaaccccacagtcggcatcggctaccagccttacagagtggtggtgctgagcttcgaactgctgaatgcccctgccacagtgtgcggccctaagctgtctacagagctggtcaagaaccagtgcgtgaacttcaacttcaacggcctgaccggcaccggcgtgctgacagatagcagcaagagattccagcctttccagcagtttggccgggatgtgtccgatttcaccgatagcgtgcgggaccccaagacactggaaatcctggacatcagcccctgcagctttggcggagtgtctgtgatcacccctggcaccaatacctctagcgaagtggccgtgctgtatcaggacgtgaactgcaccgatgtgcccacagccattcacgccgatcagctgacaccagcttggagagtgtactctaccggtgtcaacgtgttccagacacaagccggctgtctgattggagccgaacacgtgaacgccagctacgagtgcgacatccctatcggagctggcatctgtgccagctaccacacagccagcacactgagaagcaccggccagaaatccatcgtggcctacacaatgtctctgggcgccgagaactctatcgcctacagcaacaacacaatcgctatccccaccaatttcagcatcagcgtgaccaccgaagtgatgcccgtgtccatggccaagacctccgtggattgcaccatgtacatctgcggcgacagcaccgagtgcagcaacctgctgctgcagtacggcagcttctgcacccagctgaatagagccctgagcggaattgccgtggaacaggacaagaacacccgggaagtgttcgcccaagtgaagcagatgtataagacccctgccatcaaggacttcggcggctttaacttcagccagatcctgcctgatcctagcaagcccaccaagagaagcttcatcgaggacctgctgttcaacaaagtgaccctggccgacgccggctttatgaagcagtatggcgaatgcctgggcgacatcagcgccagggatctgatttgcgcccagaagtttaacggactgaccgtgctgcctcctctgctgaccgatgagatgatcgccgcctatacagccgctctggtgtctggcacagctaccgccggatggacatttggagctggcgctgctctgcagatcccctttgctatgcagatggcctacagattcaatggcatcggagtgacccagaatgtgctgtacgagaaccagaagcagatcgccaaccagttcaacaaggccatcagtcagatccaagagagcctgaccaccacaagcacagccctgggcaaactgcaggacgtggtcaaccagaatgcccaggctctgaacaccctggtcaagcagctgagcagcaacttcggcgccatcagctccgtgctgaacgatatcctgagccggctggacaaggtggaagccgaggtgcagatcgacagactgatcaccggaaggctgcagagcctgcagacctacgttacccagcagctgatcagagccgccgagattagagccagcgctaatctggccgccaccaagatgtctgagtgtgtgctgggccagagcaagagagtggacttttgcggcaagggctaccacctgatgagcttccctcaagccgctcctcacggcgtggtgtttctgcacgtgacatacgtgccaagccaagagcggaacttcaccaccgctccagccatttgccacgagggcaaagcctactttccccgcgaaggcgtgttcgtgtctaacggcacaagctggttcatcacccagcgcaacttctacagccctcagatcatcaccaccgacaacaccttcgtgtccggcaactgcgacgtcgtgatcggcatcatcaacaataccgtgtacgacccactgcagcccgagctggacagcttcaaagaggaactggacaagtacttcaagaatcacacaagccccgacgtggacctgggcgatatctctggcatcaatgcctccgtcgtgaacatccagaaagagatcgaccggctgaacgaggtggccaagaacctgaacgagtccctgatcgacctgcaagagctggggaagtacgagcaaggatctggctatattcccgaggctcctagagatggccaggcctacgttagaaaggacggcgagtgggtcctgctgagcacctttcttggaactagtctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtga

SEQ ID NO: 41 - DNA, 3843 bp, synthetic construct
  misc_feature 1..3843: note = Ancestral spike protein
  source 1..3843: mol_type = other DNA
atgttcgtgtttctggtgctgctgcctctggtgtccagccagtgtgtgaatctgaccggcagaacccctctgaaccccaactacaccaacagcagccagcggggcgtgtactaccccgacaccatctttagaagcgacaccctggtgctgagccagggctacttcctgcctttctacagcaacgtgtcctggtactacagcctgaccaccaacaacgccgccaccaagagattcgacaaccccatcctggacttcaaggacggcatctactttgccgccaccgagaagtccaacatcatcagaggctggatcttcggcaccacactggacaacacaagccagagcctgctgatcgtgaacaacgccaccaacgtgatcatcaaagtgtgcaacttccagttctgctacgacccctacctgagcggctactacggccacaacaacaagacctggtccatccgcgagttcgccgtgtacagcagctacgccaattgcaccttcgagtacgtgtccaagagcttcatgctggacatcagcggcaaaggcggcctgttcaataccctgcgcgagtttgtgttcagaaacgtggacggctacttcaagatctactccaagtacacccctgtgaacctgaaccggggcctgcctacaggcttttctgttctgcagcccctggtggaactgcccgtgggcatcaacatcaccaagttcagaaccctgctgaccatccacagaggcgaccccatgcctaacaatggctggaccgcctttagcgccgcctacttcgtgggctacctgaagcctcggacctttatgctgaagtacaacgagaacggcaccatcaccgacgccgtggattgtgcccttgatcctctgagcgagacaaagtgcaccctgaagtccctgacagtgcagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaaaggcaccggcgtgctgaccgatagcagcaagagatttcagagcttccagcagttcggccgggacgccagcgatttcacagacagcgttagagatccccagacactggaaatcctggatatcaccccttgcagcttcggcggagtgtccgtgatcacccctggcacaaataccagctctgaggtggccgtgctgtaccaggatgtgaactgcaccgatgtgcccacagccatccatgccgatcagctgacacccgcctggcggatctatagcaccggcaccaatgtgtttcagacccaagccggctgtctgattggagccgagcatgtgaatgccagctacgagtgcgacatccctatcggagccggcatctgcgccagctaccacacagcctctatcctgagatccaccagccagaaagccatcgtggcctacacaatgtctctgggcgccgagaactctatcgcctacgccaacaacagcattgctatccccaccaacttctccatcagcgtgaccaccgaagtgatgcccgtgtccatggccaagaccagcgtggactgcaccatgtacatctgcggcgacagcatcgagtgcagcaacctgctgctgcagtacggcagcttctgcacccagctgaatagagccctgaccggaatcgccatcgagcaggacaagaacacccaagaggtgttcgcccaagtgaagcagatctacaagacccctcctatcaaggacttcggcggcttcaatttcagccagatcctgccagatccaagcaagcccagcaagcggagcttcatcgaggacctgctgttcaacaaagtgacactggccgacgccggcttcatcaagcagtatggcgattgcctgggcgacatctccgccagggatctgatttgcgcccagaagttcaacggactgaccgtgctgcctcctctgctgacagatgagatgatcgccgcctataccgccgctctgatctctggaacagccaccgccggatggacatttggagctggtgcagctctgcagatccccttcgctatgcagatggcctaccggttcaatggcatcggcgtgacccagaatgtgctgtacgagaaccagaagctgattgccaaccagttcaacagcgccatcggcaagatccaagagagcctgacctctaccgccagcgctctgggaaagctgcaggacgtcgtgaaccagaacgcccaggctctgaacaccctcgtgaagcagctgagcagcaatttcggcgccatcagcagcgtgctgaacgacatcctgagccggctggataaggtggaagccgaggtgcagatcgaccggctgattacaggcagactgcagagcctccagacctacgtgacacagcagctgatcagagccgccgagattagagcctctgccaatctggccgccacaaagatgagcgagtgtgtgctgggccagagcaagagagtggacttttgcggcaagggctaccacctgatgagcttccctcagtctgctcctcacggcgtggtgtttctgcacgtgacatacgtgcccagccaagagaagaacttcaccaccgctccagccatctgccacgagggcaaagcccactttcctagagaaggcgtgttcgtgtccaacggcacccattggttcgtgactcagcggaacttctacgagccccagatcatcaccaccgacaacaccttcgtgtccggcaactgcgacgtggtcatcggcatcatcaacaataccgtgtacgacccactgcagcccgagctggacagcttcaaagaggaactggacaagtactttaagaaccacacaagccccgacgtggacctgggcgatatcagcggaatcaatgccagcgtggtcaacatccagaaagagatcgataggctgaacgaggtggccaagaacctgaatgagtccctgattgacctgcaagagctggggaagtacgagcaaggatctggctatattcccgaggctcctagagatggccaggcctacgttagaaaggacggcgagtgggtcctgctgagcacctttcttggaactagtctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtga

SEQ ID NO: 42 - DNA, 3831 bp, synthetic construct
  misc_feature 1..3831: note = Ancestral spike protein
  source 1..3831: mol_type = other DNA
atgttcgtgtttctggtgctgctgcctctggtgtccagcacagctcaagagggcacctgtggcaccatcagcaacaagacccctccaaacatgaaccagttcagcagcagcagacggggcgtgtactaccccgacgacatcttcagatccgacgtgctgcatctgacccaggactacttcctgcctttcaacagcaacgtgaccagatacctgagcctgaacgccgacagcaaccggatcgtcagattcgacaaccccatcctgccattcggcgacggcatctattttgccgccaccgagaagtccaacgtgatcagaggctggatcttcggcagcaccctggacaataccagccagagcgccatcatcgtgaacaacagcacccacatcatcatcaaagtgtgcaacttccagctgtgcgacgaccccatgttcaccgtgtctagaggccagcactacaagacctgggtgtacacaaacgcccggaactgcacctacgagtacgtgtccaagagctttcagctggacgtgtccgagaagaacggcaacttcaagcacctgagggaatttgtgttcaagaacgtggacggcttcctgcacgtgtacagcgcctacgagcctatcgacctggctagaggactgcctagcggcttctctgtgctgaagcccatcctgaagctgcccctgggcatcaacatcaccagcttcagagtcgtgatgaccatgttcagccccaccaccagcaattggctggccgaaagcgccgcctacttcgtgggatacctgaagccaaccaccttcatgctgaagttcaacgagaacggcacaatcaccgacgccgtggactgttctcaggaccctctgagcgagctgaagtgcaccctgaagtccttcaacgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaaaggcaccggcgtgctgaccgatagcagcaagagattccagagcttccagcagttcggcagggacgccagcgatttcacagacagcgtcagagatccccagacactggaaatcctggacatcagcccttgcagcttcggcggagtgtctgtgatcacccctggcacaaatgccagctctgaagtggctgtgctgtaccaggacgtgaactgtaccgatgtgcccacagccatccacgccgatcaactgacaccagcttggcgggtgtactctaccggtgtcaacgtgttccagacacaagccggctgtctgattggagccgaacacgtgaacgccagctacgagtgcgacatccctatcggagctggaatctgcgcctcctaccacacagccagcacactgagaagcaccggccagaaatccatcgtggcctacacaatgtctctgggcgccgagaactctatcgcctacgccaacaactccattgctatccccaccaacttctccatcagcgtgaccaccgaagtgatgcccgtgtccatggccaagacctccgtggattgcaccatgtacatctgcggcgacagccaagagtgcagcaacctgctgctccagtacggcagcttctgcacccagctgaatagagccctgaccggaatcgccatcgagcaggacaagaacacccaagaggtgttcgcccaagtgaagcagatgtataagacccctgccatcaaggacttcggcggcttcaatttcagccagatcctgcctgatcctagcaagcccaccaagcggagcttcatcgaggacctgctgttcaacaaagtgaccctggccgacgccggctttatgaagcagtatggcgagtgcctgggcgacatctctgccagggatctgatttgcgcccagaagtttaacggactgaccgtgctgcctcctctgctgacagatgagatgatcgccgcctataccgccgcactggtgtctggtactgctaccgccggatggacatttggagctggtgccgctctgcagatccccttcgctatgcagatggcctacagattcaacggcatcggcgtgacccagaacgtgctgtatgagaaccagaagcagatcgccaatcagttcaacaaggccatcagtcagatccaagagagcctgaccaccacaagcacagccctgggaaagctgcaggacgtggtcaaccagaatgcccaggctctgaacaccctggtcaagcagctgagcagcaatttcggcgccatcagcagcgtgctgaacgacatcctgagccggctggataaggtggaagccgaggtgcagatcgaccggctgattacaggcagactgcagagcctgcagacctacgtgacacagcagctgatcagagccgccgagattagagcctctgccaatctggccgccaccaagatgagcgagtgtgtgctgggccagagcaagagagtggacttttgcggcaagggctaccacctgatgagcttccctcaagccgctcctcacggcgtggtgtttctgcacgtgacatacgtgccaagccaagagcggaacttcaccaccgctccagccatttgccacgagggcaaagcctactttccccgcgaaggcgtgttcgtgtctaacggcacctcctggttcatcacccagaggaacttctacagccctcagatcatcaccaccgacaacaccttcgtggccggcaattgcgacgtcgtgatcggcatcatcaacaataccgtgtacgaccctctgcagcccgagctggacagcttcaaagaggaactggacaagtacttcaagaatcacacaagccccgacgtggacctgggcgatatcagcggaatcaatgccagcgtcgtgaacatccagaaagagatcgacagactgaacgaggtggccaagaacctgaacgagtccctgattgacctgcaagagctggggaagtacgagcaaggatctggctatattcccgaggctcctagagatggccaggcctacgttagaaaggacggcgagtgggtcctgctgagcacctttcttggaactagtctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtga

SEQ ID NO: 43 - DNA, 3837 bp, synthetic construct
  misc_feature 1..3837: note = Ancestral spike protein
  source 1..3837: mol_type = other DNA
atgttcgtgtttctggtgctgctgcccctggtgtcctctacagctcaagagggcacatgtggcaccctgagcaacaagagccctcctaacatgacccagttcagcagctctcggagaggcgtgtactaccccgacgacatcttcagatccgacgtgctgcatctgacccaggactacttcctgcctttcaacagcaacgtgaccagatacctgagcctgaacagcgacagcgaccggatcgtcagattcgacaaccctatcatccccttcggcgacggggtgtactttgccgccaccgagaagtccaacgtgatcagaggctggatcttcggcagcaccctggacaataccagccagagcgccatcatcatgaacaacagcacccacatcgtgattcgcgtgtgcaacttccagctgtgcgacgaccctatgttcgccgtgtctagacctaccggccagcactacaagacctggatctacaccaacgccagaaactgcacctacgagtacgtgtccaagagctttcagctggacgtgtccgagaagcccggcaacttcaaacacctgagggaatttgtgttcaagaacgtggacggcttcctgcacgtgtacagcggctacgagcctatcgatgtggccagaggactgcctagcggcttctctgtgctgaagcccatcttcaagctgcctctgggcatcaacatcaccaacttcagagtgatcatgaccatgttcagccccaccaccagcaactggggagctgaagccgccgcttacttcgtgggctacctgaagcctaccaccttcatgctgaagttcgacgagaacggcaccatcaccgacgccgtggactgtagccaagatcctctgagcgagctgaagtgcaccgtgaagtccttcaacgtggaaaagggcatctaccagaccagcaatttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagatagcagcaagagattccagcctttccagcagtttggccgggatgtgtccgatttcaccgatagcgtgcgggaccccaagacactggaaatcctggacatcagcccctgcagctttggcggagtgtctgtgatcacccctggcaccaatacctctagcgaagtggccgtgctgtatcaggacgtgaactgcaccgatgtgcccacagccattcacgccgatcagctgacaccagcttggagagtgtactctaccggtgtcaacgtgttccagacacaagccggctgtctgattggagccgaacacgtgaacgccagctacgagtgcgacatccctatcggagctggcatctgtgccagctaccacacagccagcacactgagaagcaccggccagaaatccatcgtggcctacacaatgtctctgggcgccgagaactctatcgcctacagcaacaacacaatcgctatccccaccaatttcagcatcagcgtgaccaccgaagtgatgcccgtgtccatggccaagacctccgtggattgcaccatgtacatctgcggcgacagcaccgagtgcagcaacctgctgctgcagtacggcagcttctgcacccagctgaatagagccctgagcggaattgccgtggaacaggacaagaacacccgggaagtgttcgcccaagtgaagcagatgtataagacccctgccatcaaggacttcggcggctttaacttcagccagatcctgcctgatcctagcaagcccaccaagagaagcttcatcgaggacctgctgttcaacaaagtgaccctggccgacgccggctttatgaagcagtatggcgaatgcctgggcgacatcagcgccagggatctgatttgcgcccagaagtttaacggactgaccgtgctgcctcctctgctgaccgatgagatgatcgccgcctatacagccgctctggtgtctggcacagctaccgccggatggacatttggagctggcgctgctctgcagatcccctttgctatgcagatggcctacagattcaatggcatcggagtgacccagaatgtgctgtacgagaaccagaagcagatcgccaaccagttcaacaaggccatcagtcagatccaagagagcctgaccaccacaagcacagccctgggcaaactgcaggacgtggtcaaccagaatgcccaggctctgaacaccctggtcaagcagctgagcagcaacttcggcgccatcagctccgtgctgaacgatatcctgagccggctggacaaggtggaagccgaggtgcagatcgacagactgatcaccggaaggctgcagagcctgcagacctacgttacccagcagctgatcagagccgccgagattagagccagcgctaatctggccgccaccaagatgtctgagtgtgtgctgggccagagcaagagagtggacttttgcggcaagggctaccacctgatgagcttccctcaagccgctcctcacggcgtggtgtttctgcacgtgacatacgtgccaagccaagagcggaacttcaccaccgctccagccatttgccacgagggcaaagcctactttccccgcgaaggcgtgttcgtgtctaacggcacaagctggttcatcacccagcgcaacttctacagccctcagatcatcaccaccgacaacaccttcgtgtccggcaactgcgacgtcgtgatcggcatcatcaacaataccgtgtacgacccactgcagcccgagctggacagcttcaaagaggaactggacaagtacttcaagaatcacacaagccccgacgtggacctgggcgatatctctggcatcaatgcctccgtcgtgaacatccagaaagagatcgaccggctgaacgaggtggccaagaacctgaacgagtccctgatcgacctgcaagagctggggaagtacgagcaaggatctggctatattcccgaggctcctagagatggccaggcctacgttagaaaggacggcgagtgggtcctgctgagcacctttcttggaactagtctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtga

SEQ ID NO: 44 - protein, 1288 aa, synthetic construct
  REGION 1..1288: note = Mutated coronavirus spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 45 - protein, 574 aa, Human orthopneumovirus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 46 - protein, 29 aa, Human orthopneumovirus
RRELPRFMNYTLNNAKKTNVTLSKKRKRR

SEQ ID NO: 47 - protein, 17 aa, Human orthopneumovirus
KNYIDKQLLPIVNKQSC

SEQ ID NO: 48 - protein, 25 aa, Human orthopneumovirus
MELLILKANAITTILTAVTFCFASG

SEQ ID NO: 49 - protein, 593 aa, synthetic construct
  REGION 1..593: note = Ancestral RS virus fusion glycoprotein
MELLILKANAITTILTAVTFCFASGQNITEEFYQSTCSAVSRGYLSALRTGWYTSVITIELSNIKENKCNGTDTKVKLIKQELDKYKNAVTELQLLMQNTPATNNRARREAPRFMNYTLNTTKNLNVSISKKRKRRFLGFLLGVGSAIASGIAVSKVLHLEGEVNKIKNALLSTNKAVVSLSNGVSVLTSKVLDLKNYIDKQLLPIVNKQSCRISNIETVIEFQQKNNRLLEITREFSVNAGVTTPLSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNTDIFNSKYDCKIMTSKTDISSSVITSLGAIVSCYGKTKCTASNKNRGIIKTFSNGCDYVSNKGVDTVSVGNTLYYVNKLEGKSLYVKGEPIINYYDPLVFPSDEFDASISQVNEKINQSLAFIRRSDELLGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 50 - protein, 513 aa, synthetic construct
  REGION 1..513: note = Ancestral RS virus fusion glycoprotein
MELLILKANAITTILTAVTFCFASGQNITEEFYQSTCSAVSRGYLSALRTGWYTSVITIELSNIKENKCNGTDTKVKLIKQELDKYKNAVTELQLLMQNTPATNNRARREAPRFMNYTLNTTKNLNVSISKKRKRRFLGFLLGVGSAIASGIAVSKVLHLEGEVNKIKNALLSTNKAVVSLSNGVSVLTSKVLDLKNYIDKQLLPIVNKQSCRISNIETVIEFQQKNNRLLEITREFSVNAGVTTPLSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNTDIFNSKYDCKIMTSKTDISSSVITSLGAIVSCYGKTKCTASNKNRGIIKTFSNGCDYVSNKGVDTVSVGNTLYYVNKLEGKSLYVKGEPIINYYDPLVFPSDEFDASISQVNEKINQSLAFIRRSDELL

SEQ ID NO: 51 - protein, 488 aa, synthetic construct
  REGION 1..488: note = Ancestral RS virus fusion glycoprotein
QNITEEFYQSTCSAVSRGYLSALRTGWYTSVITIELSNIKENKCNGTDTKVKLIKQELDKYKNAVTELQLLMQNTPATNNRARREAPRFMNYTLNTTKNLNVSISKKRKRRFLGFLLGVGSAIASGIAVSKVLHLEGEVNKIKNALLSTNKAVVSLSNGVSVLTSKVLDLKNYIDKQLLPIVNKQSCRISNIETVIEFQQKNNRLLEITREFSVNAGVTTPLSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNTDIFNSKYDCKIMTSKTDISSSVITSLGAIVSCYGKTKCTASNKNRGIIKTFSNGCDYVSNKGVDTVSVGNTLYYVNKLEGKSLYVKGEPIINYYDPLVFPSDEFDASISQVNEKINQSLAFIRRSDELL

SEQ ID NO: 52 - protein, 82 aa, synthetic construct
  REGION 1..82: note = F2 part of ancestral RS virus fusion glycoprotein
QNITEEFYQSTCSAVSRGYLSALRTGWYTSVITIELSNIKENKCNGTDTKVKLIKQELDKYKNAVTELQLLMQNTPATNNRA

SEQ ID NO: 53 - protein, 29 aa, synthetic construct
  REGION 1..29: note = Maturation peptide
RREAPRFMNYTLNTTKNLNVSISKKRKRR

SEQ ID NO: 54 - protein, 377 aa, synthetic construct
  REGION 1..377: note = F1 part of ancestral RS virus fusion glycoprotein
FLGFLLGVGSAIASGIAVSKVLHLEGEVNKIKNALLSTNKAVVSLSNGVSVLTSKVLDLKNYIDKQLLPIVNKQSCRISNIETVIEFQQKNNRLLEITREFSVNAGVTTPLSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNTDIFNSKYDCKIMTSKTDISSSVITSLGAIVSCYGKTKCTASNKNRGIIKTFSNGCDYVSNKGVDTVSVGNTLYYVNKLEGKSLYVKGEPIINYYDPLVFPSDEFDASISQVNEKINQSLAFIRRSDELL

SEQ ID NO: 55 - protein, 583 aa, synthetic construct
  REGION 1..583: note = Ancestral RS virus fusion glycoprotein
MELLILKANAITTILTAVTFCFASGQNITEEFYQSTCSAVSRGYLSALRTGWYTSVITIELSNIQKNKCNSTDSKVKLIKQELDRYKNAVTELQLLMQNTPATNNRARRETPRFMGYLLGKKRKRRFLGFLLGVGSAIASGVAVSKVLHLEGEVNKIKNALLSTNKAVVSLSNGVSVLTSKVLDLKNYIDKELLPKVNKHSCRISNIETVIEFQQKNNRLLEITREFSVNAGVTTPLSTYMLTNSELLSLINDMPITNDQKKLMSSNVQIVRQQSYSIMSIVKEEVLAYVVQLPLYGVIDTPCWKLHTSPLCTTNNKEGSNICLTRTDRGWYCDNAGSVSFFPQAETCKVQSNRVFCDTMNSLTLPTEVNLCNTDIFNSKYDCKIMTSKTDISSSVITSLGAIVSCYGKTKCTASNKNRGIIKTFSNGCDYVSNKGVDTVSVGNTLYYVNKLEGKSLYVKGEPIINYYDPLVFPSDEFDASISQVNEKINQSLAFIRRSDELLGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 56 - protein, 503 aa, synthetic construct
  REGION 1..503: note = Ancestral RS virus fusion glycoprotein
MELLILKANAITTILTAVTFCFASGQNITEEFYQSTCSAVSRGYLSALRTGWYTSVITIELSNIQKNKCNSTDSKVKLIKQELDRYKNAVTELQLLMQNTPATNNRARRETPRFMGYLLGKKRKRRFLGFLLGVGSAIASGVAVSKVLHLEGEVNKIKNALLSTNKAVVSLSNGVSVLTSKVLDLKNYIDKELLPKVNKHSCRISNIETVIEFQQKNNRLLEITREFSVNAGVTTPLSTYMLTNSELLSLINDMPITNDQKKLMSSNVQIVRQQSYSIMSIVKEEVLAYVVQLPLYGVIDTPCWKLHTSPLCTTNNKEGSNICLTRTDRGWYCDNAGSVSFFPQAETCKVQSNRVFCDTMNSLTLPTEVNLCNTDIFNSKYDCKIMTSKTDISSSVITSLGAIVSCYGKTKCTASNKNRGIIKTFSNGCDYVSNKGVDTVSVGNTLYYVNKLEGKSLYVKGEPIINYYDPLVFPSDEFDASISQVNEKINQSLAFIRRSDELL

SEQ ID NO: 57 - protein, 478 aa, synthetic construct
  REGION 1..478: note = Ancestral RS virus fusion glycoprotein
QNITEEFYQSTCSAVSRGYLSALRTGWYTSVITIELSNIQKNKCNSTDSKVKLIKQELDRYKNAVTELQLLMQNTPATNNRARRETPRFMGYLLGKKRKRRFLGFLLGVGSAIASGVAVSKVLHLEGEVNKIKNALLSTNKAVVSLSNGVSVLTSKVLDLKNYIDKELLPKVNKHSCRISNIETVIEFQQKNNRLLEITREFSVNAGVTTPLSTYMLTNSELLSLINDMPITNDQKKLMSSNVQIVRQQSYSIMSIVKEEVLAYVVQLPLYGVIDTPCWKLHTSPLCTTNNKEGSNICLTRTDRGWYCDNAGSVSFFPQAETCKVQSNRVFCDTMNSLTLPTEVNLCNTDIFNSKYDCKIMTSKTDISSSVITSLGAIVSCYGKTKCTASNKNRGIIKTFSNGCDYVSNKGVDTVSVGNTLYYVNKLEGKSLYVKGEPIINYYDPLVFPSDEFDASISQVNEKINQSLAFIRRSDELL

SEQ ID NO: 58 - protein, 82 aa, synthetic construct
  REGION 1..82: note = F2 part of ancestral RS virus fusion glycoprotein
QNITEEFYQSTCSAVSRGYLSALRTGWYTSVITIELSNIQKNKCNSTDSKVKLIKQELDRYKNAVTELQLLMQNTPATNNRA

SEQ ID NO: 59 - protein, 19 aa, synthetic construct
  REGION 1..19: note = Maturation peptide
RRETPRFMGYLLGKKRKRR

SEQ ID NO: 60 - protein, 377 aa, synthetic construct
  REGION 1..377: note = F1 part of ancestral RS virus fusion glycoprotein
FLGFLLGVGSAIASGVAVSKVLHLEGEVNKIKNALLSTNKAVVSLSNGVSVLTSKVLDLKNYIDKELLPKVNKHSCRISNIETVIEFQQKNNRLLEITREFSVNAGVTTPLSTYMLTNSELLSLINDMPITNDQKKLMSSNVQIVRQQSYSIMSIVKEEVLAYVVQLPLYGVIDTPCWKLHTSPLCTTNNKEGSNICLTRTDRGWYCDNAGSVSFFPQAETCKVQSNRVFCDTMNSLTLPTEVNLCNTDIFNSKYDCKIMTSKTDISSSVITSLGAIVSCYGKTKCTASNKNRGIIKTFSNGCDYVSNKGVDTVSVGNTLYYVNKLEGKSLYVKGEPIINYYDPLVFPSDEFDASISQVNEKINQSLAFIRRSDELL

SEQ ID NO: 61 - protein, 17 aa, synthetic construct
  REGION 1..17: note = Antigenic site
KNYIDKELLPKVNKHSC

SEQ ID NO: 62 - protein, 573 aa, synthetic construct
  REGION 1..573: note = Ancestral RS virus fusion glycoprotein
MELLILKANAITTILTAVTFCFASGNNLTEKFYESTCSVVTRGYKSALRTGWYTSVMTIELSQSNIENCKCSGTDDSNSLIKQELELYKNAVDELRTLSSDALKNRARRRSPRFLGFILGGFALGVGSAVTAGVALAKTIQLEGEVAKIKDALRNTNEAVVSLSNGISVLATAVNDLKNFISKELLPKINKNSCDISDIKTVIRFQQYNKRLLEVTREFSSNAGITTAVSTYMLTDSELVSLVNDMPVSSGQKKLMLSNRAIVRRKGFAILSSVNADTLVYVVQLPLFGVIDTPCWKIRSSPLCSNNNDEYACLARADQGWYCHNAGSISYFPNPEDCEIQNNYVFCDTMNSLTVPTESRECNSNIYTTKYDCKISTSKTDISTAVLTSLGALVSCYGHTSCTVSNNNKGIIKTLSNGCHYISNKGVDTVSVGNTVYYLSKLEGKSLYVKGEPIVNNYDPLSFPDDEFDVAISQVNESINQSRSFIKRSDELLGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 63 - protein, 493 aa, synthetic construct
  REGION 1..493: note = Ancestral RS virus fusion glycoprotein
MELLILKANAITTILTAVTFCFASGNNLTEKFYESTCSVVTRGYKSALRTGWYTSVMTIELSQSNIENCKCSGTDDSNSLIKQELELYKNAVDELRTLSSDALKNRARRRSPRFLGFILGGFALGVGSAVTAGVALAKTIQLEGEVAKIKDALRNTNEAVVSLSNGISVLATAVNDLKNFISKELLPKINKNSCDISDIKTVIRFQQYNKRLLEVTREFSSNAGITTAVSTYMLTDSELVSLVNDMPVSSGQKKLMLSNRAIVRRKGFAILSSVNADTLVYVVQLPLFGVIDTPCWKIRSSPLCSNNNDEYACLARADQGWYCHNAGSISYFPNPEDCEIQNNYVFCDTMNSLTVPTESRECNSNIYTTKYDCKISTSKTDISTAVLTSLGALVSCYGHTSCTVSNNNKGIIKTLSNGCHYISNKGVDTVSVGNTVYYLSKLEGKSLYVKGEPIVNNYDPLSFPDDEFDVAISQVNESINQSRSFIKRSDELL

SEQ ID NO: 64 - protein, 468 aa, synthetic construct
  REGION 1..468: note = Ancestral RS virus fusion glycoprotein
NNLTEKFYESTCSVVTRGYKSALRTGWYTSVMTIELSQSNIENCKCSGTDDSNSLIKQELELYKNAVDELRTLSSDALKNRARRRSPRFLGFILGGFALGVGSAVTAGVALAKTIQLEGEVAKIKDALRNTNEAVVSLSNGISVLATAVNDLKNFISKELLPKINKNSCDISDIKTVIRFQQYNKRLLEVTREFSSNAGITTAVSTYMLTDSELVSLVNDMPVSSGQKKLMLSNRAIVRRKGFAILSSVNADTLVYVVQLPLFGVIDTPCWKIRSSPLCSNNNDEYACLARADQGWYCHNAGSISYFPNPEDCEIQNNYVFCDTMNSLTVPTESRECNSNIYTTKYDCKISTSKTDISTAVLTSLGALVSCYGHTSCTVSNNNKGIIKTLSNGCHYISNKGVDTVSVGNTVYYLSKLEGKSLYVKGEPIVNNYDPLSFPDDEFDVAISQVNESINQSRSFIKRSDELL

SEQ ID NO: 65 - protein, 82 aa, synthetic construct
  REGION 1..82: note = F2 part of ancestral RS virus fusion glycoprotein
NNLTEKFYESTCSVVTRGYKSALRTGWYTSVMTIELSQSNIENCKCSGTDDSNSLIKQELELYKNAVDELRTLSSDALKNRA

SEQ ID NO: 66 - protein, 13 aa, synthetic construct
  REGION 1..13: note = Maturation peptide
RRRSPRFLGFILG

SEQ ID NO: 67 - protein, 373 aa, synthetic construct
  REGION 1..373: note = F1 part of ancestral RS virus fusion protein
GFALGVGSAVTAGVALAKTIQLEGEVAKIKDALRNTNEAVVSLSNGISVLATAVNDLKNFISKELLPKINKNSCDISDIKTVIRFQQYNKRLLEVTREFSSNAGITTAVSTYMLTDSELVSLVNDMPVSSGQKKLMLSNRAIVRRKGFAILSSVNADTLVYVVQLPLFGVIDTPCWKIRSSPLCSNNNDEYACLARADQGWYCHNAGSISYFPNPEDCEIQNNYVFCDTMNSLTVPTESRECNSNIYTTKYDCKISTSKTDISTAVLTSLGALVSCYGHTSCTVSNNNKGIIKTLSNGCHYISNKGVDTVSVGNTVYYLSKLEGKSLYVKGEPIVNNYDPLSFPDDEFDVAISQVNESINQSRSFIKRSDELL

SEQ ID NO: 68 - protein, 17 aa, synthetic construct
  REGION 1..17: note = Antigenic site
KNFISKELLPKINKNSC

SEQ ID NO: 69 - protein, 575 aa, synthetic construct
  REGION 1..575: note = Ancestral RS virus fusion glycoprotein
MELLILKANAITTILTAVTFCFASGGNLTESYLEETCSTVTRGYKSALRTGWYTNVMTIELSQGNIENCTCSGTNDSNSLIRQELELTKNALEELKTVSADQLAKEARLRSPRFLGFVLGGIALGVATAAAVTAGVALAKTIRLEGEVAAIKNALRNTNEAVSTLSNGVRVLATAVNDLKDFISKELLPAINKNSCDISDIKMAISFQQYNRRLLNVVREFSSNAGITPAVSLYMLTDAELVSLVNNMPTSSGQIKLMLENRAIVRRKGFAILIGVYGDTLVYMVQLPIFGVIDTPCWKVRASPLCSNKDGEYACLLREDQGWYCQNAGSIAYYPNEEDCEVRNDYVFCDTAASLTVPSESEECNRNIFTTKYDCKVSTSKTPISTAVLTPLGALVSCYGGVSCSVGNNKKGIIKQLNKGCSYISNKGADTVTIDNTVYQLSKVEGKSHTIKGEPVVNNYDPLSFPEDEFNVALDQVNESIDKSKNFIDKSDELLGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 70 - protein, 495 aa, synthetic construct
  REGION 1..495: note = Ancestral RS virus fusion glycoprotein
MELLILKANAITTILTAVTFCFASGGNLTESYLEETCSTVTRGYKSALRTGWYTNVMTIELSQGNIENCTCSGTNDSNSLIRQELELTKNALEELKTVSADQLAKEARLRSPRFLGFVLGGIALGVATAAAVTAGVALAKTIRLEGEVAAIKNALRNTNEAVSTLSNGVRVLATAVNDLKDFISKELLPAINKNSCDISDIKMAISFQQYNRRLLNVVREFSSNAGITPAVSLYMLTDAELVSLVNNMPTSSGQIKLMLENRAIVRRKGFAILIGVYGDTLVYMVQLPIFGVIDTPCWKVRASPLCSNKDGEYACLLREDQGWYCQNAGSIAYYPNEEDCEVRNDYVFCDTAASLTVPSESEECNRNIFTTKYDCKVSTSKTPISTAVLTPLGALVSCYGGVSCSVGNNKKGIIKQLNKGCSYISNKGADTVTIDNTVYQLSKVEGKSHTIKGEPVVNNYDPLSFPEDEFNVALDQVNESIDKSKNFIDKSDELL

SEQ ID NO: 71 - protein, 470 aa, synthetic construct
  REGION 1..470: note = Ancestral RS virus fusion glycoprotein
GNLTESYLEETCSTVTRGYKSALRTGWYTNVMTIELSQGNIENCTCSGTNDSNSLIRQELELTKNALEELKTVSADQLAKEARLRSPRFLGFVLGGIALGVATAAAVTAGVALAKTIRLEGEVAAIKNALRNTNEAVSTLSNGVRVLATAVNDLKDFISKELLPAINKNSCDISDIKMAISFQQYNRRLLNVVREFSSNAGITPAVSLYMLTDAELVSLVNNMPTSSGQIKLMLENRAIVRRKGFAILIGVYGDTLVYMVQLPIFGVIDTPCWKVRASPLCSNKDGEYACLLREDQGWYCQNAGSIAYYPNEEDCEVRNDYVFCDTAASLTVPSESEECNRNIFTTKYDCKVSTSKTPISTAVLTPLGALVSCYGGVSCSVGNNKKGIIKQLNKGCSYISNKGADTVTIDNTVYQLSKVEGKSHTIKGEPVVNNYDPLSFPEDEFNVALDQVNESIDKSKNFIDKSDELL

SEQ ID NO: 72 - protein, 82 aa, synthetic construct
  REGION 1..82: note = F2 part of ancestral RS virus fusion glycoprotein
GNLTESYLEETCSTVTRGYKSALRTGWYTNVMTIELSQGNIENCTCSGTNDSNSLIRQELELTKNALEELKTVSADQLAKEA

SEQ ID NO: 73 - protein, 13 aa, synthetic construct
  REGION 1..13: note = Maturation peptide
RLRSPRFLGFVLG

SEQ ID NO: 74 - protein, 375 aa, synthetic construct
  REGION 1..375: note = F1 part of ancestral RS virus fusion glycoprotein
GIALGVATAAAVTAGVALAKTIRLEGEVAAIKNALRNTNEAVSTLSNGVRVLATAVNDLKDFISKELLPAINKNSCDISDIKMAISFQQYNRRLLNVVREFSSNAGITPAVSLYMLTDAELVSLVNNMPTSSGQIKLMLENRAIVRRKGFAILIGVYGDTLVYMVQLPIFGVIDTPCWKVRASPLCSNKDGEYACLLREDQGWYCQNAGSIAYYPNEEDCEVRNDYVFCDTAASLTVPSESEECNRNIFTTKYDCKVSTSKTPISTAVLTPLGALVSCYGGVSCSVGNNKKGIIKQLNKGCSYISNKGADTVTIDNTVYQLSKVEGKSHTIKGEPVVNNYDPLSFPEDEFNVALDQVNESIDKSKNFIDKSDELL

SEQ ID NO: 75 - protein, 17 aa, synthetic construct
  REGION 1..17: note = Antigenic site
KDFISKELLPAINKNSC

SEQ ID NO: 76 - protein, 567 aa, synthetic construct
  REGION 1..567: note = Ancestral RS virus fusion glycoprotein
MELLILKANAITTILTAVTFCFASGGQLDFSKLSKIGVIKTKSYELKIYTNSTTSYIVIKLIPNLSNLKNCSKDTIEEYNKLLNRILSPIKDALERMRNSIQDRKGSGSSRRQKRFFGAIIGGVALGVATAAQITAGVALAKAQQNAKNILKLKDAIKKTNEAVQKLQDAAQQLAIAIQAIQDYINNEIIPTINQLSCEVAGLKLGIKLSQYYTELTTVFGNNITNPALSPLSIQALYNLFGGNLTELLNKLGASNEDLYSLLESGSIKGQIIDVDLEDYLIVLQVKIPTLSEIPGARIQELTSISYNTDGQEWMAVVPKYVLTRGSLISNIDISDCTITDNSVFCSRNTAYPLPPEMQECLRGNVSKCPYTKVVGSLVPRFATIDGSIVANCRSITCRCQDPPQTISQDPDQPITIIDQELCKEIQIDGITFRLSKRLSSTYYRNTNISLGQPVSLDPLDISNELGKVNQSLKESKDYIEKSNEILGSGYIPEAPRDGQAYVRKDGEWVLLSTFLGTSLEVLFQGPGHHHHHHHHSAWSHPQFEKGGGSGGGGSGGSAWSHPQFEK

SEQ ID NO: 77 - protein, 487 aa, synthetic construct
  REGION 1..487: note = Ancestral RS virus fusion glycoprotein
MELLILKANAITTILTAVTFCFASGGQLDFSKLSKIGVIKTKSYELKIYTNSTTSYIVIKLIPNLSNLKNCSKDTIEEYNKLLNRILSPIKDALERMRNSIQDRKGSGSSRRQKRFFGAIIGGVALGVATAAQITAGVALAKAQQNAKNILKLKDAIKKTNEAVQKLQDAAQQLAIAIQAIQDYINNEIIPTINQLSCEVAGLKLGIKLSQYYTELTTVFGNNITNPALSPLSIQALYNLFGGNLTELLNKLGASNEDLYSLLESGSIKGQIIDVDLEDYLIVLQVKIPTLSEIPGARIQELTSISYNTDGQEWMAVVPKYVLTRGSLISNIDISDCTITDNSVFCSRNTAYPLPPEMQECLRGNVSKCPYTKVVGSLVPRFATIDGSIVANCRSITCRCQDPPQTISQDPDQPITIIDQELCKEIQIDGITFRLSKRLSSTYYRNTNISLGQPVSLDPLDISNELGKVNQSLKESKDYIEKSNEIL

SEQ ID NO: 78 - protein, 462 aa, synthetic construct
  REGION 1..462: note = Ancestral RS virus fusion glycoprotein
GQLDFSKLSKIGVIKTKSYELKIYTNSTTSYIVIKLIPNLSNLKNCSKDTIEEYNKLLNRILSPIKDALERMRNSIQDRKGSGSSRRQKRFFGAIIGGVALGVATAAQITAGVALAKAQQNAKNILKLKDAIKKTNEAVQKLQDAAQQLAIAIQAIQDYINNEIIPTINQLSCEVAGLKLGIKLSQYYTELTTVFGNNITNPALSPLSIQALYNLFGGNLTELLNKLGASNEDLYSLLESGSIKGQIIDVDLEDYLIVLQVKIPTLSEIPGARIQELTSISYNTDGQEWMAVVPKYVLTRGSLISNIDISDCTITDNSVFCSRNTAYPLPPEMQECLRGNVSKCPYTKVVGSLVPRFATIDGSIVANCRSITCRCQDPPQTISQDPDQPITIIDQELCKEIQIDGITFRLSKRLSSTYYRNTNISLGQPVSLDPLDISNELGKVNQSLKESKDYIEKSNEIL

SEQ ID NO: 79 - protein, 85 aa, synthetic construct
  REGION 1..85: note = F2 part of ancestral RS virus fusion glycoprotein
GQLDFSKLSKIGVIKTKSYELKIYTNSTTSYIVIKLIPNLSNLKNCSKDTIEEYNKLLNRILSPIKDALERMRNSIQDRKGSGSS

SEQ ID NO: 80 - protein, 13 aa, synthetic construct
  REGION 1..13: note = Maturation peptide
SRRQKRFFGAIIG

SEQ ID NO: 81 - protein, 365 aa, synthetic construct
  REGION 1..365: note = F1 part of ancestral RS virus fusion glycoprotein
GVALGVATAAQITAGVALAKAQQNAKNILKLKDAIKKTNEAVQKLQDAAQQLAIAIQAIQDYINNEIIPTINQLSCEVAGLKLGIKLSQYYTELTTVFGNNITNPALSPLSIQALYNLFGGNLTELLNKLGASNEDLYSLLESGSIKGQIIDVDLEDYLIVLQVKIPTLSEIPGARIQELTSISYNTDGQEWMAVVPKYVLTRGSLISNIDISDCTITDNSVFCSRNTAYPLPPEMQECLRGNVSKCPYTKVVGSLVPRFATIDGSIVANCRSITCRCQDPPQTISQDPDQPITIIDQELCKEIQIDGITFRLSKRLSSTYYRNTNISLGQPVSLDPLDISNELGKVNQSLKESKDYIEKSNEIL

SEQ ID NO: 82 - protein, 17 aa, synthetic construct
  REGION 1..17: note = Antigenic site
QDYINNEIIPTINQLSC

SEQ ID NO: 83 - protein, 8 aa, Human orthopneumovirus
SNIKENKC

SEQ ID NO: 84 - protein, 8 aa, synthetic construct
  REGION 1..8: note = Antigenic site
SNIQKNKC

SEQ ID NO: 85 - protein, 8 aa, synthetic construct
  REGION 1..8: note = Antigenic site
SNIENCKC

SEQ ID NO: 86 - protein, 8 aa, synthetic construct
  REGION 1..8: note = Antigenic site
GNIENCTC

SEQ ID NO: 87 - protein, 8 aa, synthetic construct
  REGION 1..8: note = Antigenic site
SNLKNCSK